(19) United States
(12) Patent Application Publication
(10) Pub. No.: US 2024/0279726 A1
(43) Pub. Date: Aug. 22, 2024

(54) A METHOD TO AMPLIFY AND DETECT TARGET NUCLEIC ACIDS WITH VERY HIGH SPECIFICITY AND SENSITIVITY

(71) Applicant: OBRONT BIOTECH PRIVATE LIMITED, Hyderabad, Telangana (IN)

(72) Inventor: Amirul ISLAM, Hyderabad (IN)

(21) Appl. No.: 18/571,481

(22) PCT Filed: Jun. 18, 2022
(86) PCT No.: PCT/IN2022/050556

§ 371 Date: Dec. 18, 2023

(30) Foreign Application Priority Data
Jun. 19, 2021 (IN) 202141027518

(51) Int. Cl. C12Q 1/6848; C12Q 1/6818

(52) U.S. Cl. CPC C12Q 1/6848; C12Q 1/6818

(57) ABSTRACT

A method of nucleic acid target detection by nucleic acid amplification is provided including: providing at least one target nucleic acid, at least one oligonucleotide non-extendable or extendable, labelled with at least one shining group/moiety adapted to shine extra upon hybridization or getting incorporated into a nucleic acid molecule; providing at least one extendable oligonucleotide labelled with at least one converter or acceptor group/moiety adapted to convert the color of the shining group/moiety to a different colour or thermalise it, is incorporated into an amplification product; hybridizing or incorporating into a target nucleic acid amplification product the shining group/moiety of the shining group labelled oligonucleotide whereby the shining group/moiety shines extra with the extra shine being a measure of the target amplification. In the method any non-specific amplification does not generate any detectable signal under controlled attenuation by selectively controlling the removing only of the extra shine of the shining group.

### CROSS-REFERENCE TO RELATED APPLICATIONS

This application claims priority to PCT Application No. PCT/IN2022/050556, having a filing date of Jun. 18, 2022, which is based on Indian Application No. 202141027518, having a filing date of Jun. 19, 2021, the entire contents both of which are hereby incorporated by reference.

### SEQUENCE LISTING

This application includes a separate sequence listing in compliance with the requirement of 37 C.F.R.§.§ 1.824(a)(2)—1.824 (a)(6) and 1.824 (b), submitted under the file name “0035US01_SequenceListing”, created on Dec. 18, 2023, having a file size of 6.83 KB, the contents of which are hereby incorporated by reference.

### FIELD OF TECHNOLOGY

The following relates to a process for nucleic acid amplification. More specifically, the process involving Libra primer pairs and Libra probe—primer pairs amplify and detect target nucleic acids with very high specificity and sensitivity.

### BACKGROUND

Deoxyribonucleic acid (DNA) the hereditary (genetic) material present in all living organisms and cells is the blue print of all properties and abilities of them. Because of this, genetic material has been envisaged for identifying a living organism or virus or any special property of it, or determining the genotype variation or molecular analysis of diseases or defects of living organisms. As the quantity of Nucleic acid in cells and available samples of organs and tissues of living organisms is less, detection or analysis of the nucleic acid needs amplification of its quantity for its detection and/or quantification. Many nucleic acid amplification technologies have been developed in the last four decades. Polymerase chain reaction (PCR) is the most important and the first amplification technology to be developed, U.S. Pat. Nos. 4,683,195; 4,683,202; and U.S. Pat. No. 4,965,188; Saiki et al., 1985, Science 230:1350-1354, LDR and PCR, U.S. Pat. No. 6,797,470, Isothermal amplification reactions or procedure using one or more RNA Polymerases (PCT Publication No. WO 2006/081222), Strand Displacement amplification U.S. Pat. No. RE39,007E, Ligase Chain Reaction(LCR)(Wu, et al Genomics 4: 560-569(1990), Barany, et al. Proc. Natl. Acad. Sci. USA 88: 189-193 (1991),Q$ RNA Replicase System PCT Publication no WO/1994/016108, Rolling circle amplification(RCA), U.S. Pat. No. 5,854,033, NASBA (U.S. Pat. No. 5,130,238), Loop Mediated Amplification(LAMP), Nucleic Acids Research—, Recombinase Polymerase Amplification(RPA), helicase Polymerase Amplification (HPA) among others each of which is incorporated herein by reference in entirety.

Much chemistry has also been developed for the generations of measurable/quantifiable signal and among them fluorescence-based chemistries have been the chemistry of choice and most important among them include Taqman Chemistry (U.S. Pat. No. 5,538,848) that uses a fluorophore and quencher dual labelled hydrolysis probe, Scorpion primer method where one of the primers include a dual labelled stem structure probe and PCR blocker element hexaethylene monomer (Whitcombe, et al Nat Biotech. 17: 804-807(1999), U.S. Pat. No. 6,589,743, Solinas et al Nucleic Acids Res 29: E96,2001,), Intercalating dye ethidium bromide that fluoresce more intensely when intercalated into double stranded DNA than when bound to single stranded DNA or RNA (U.S. Pat. Nos. 5,994,056; 6,171,785, 6,814,934), intercalating dye SYBR Green1 [Ririe et al., Analytical Biochem. 245: 154 (1997)] Molecular Beacons (U.S. Pat. Nos. 5,925,517 and 6,103,476, 6,485,901, 6,355,421 and 6,593,091, LUX primers Nazarenko et al Nucleic Acids Res 30:e37, 2002, Donor fluorophore and acceptor fluorophore labelled linear primer pair that generates FRET signal (WO 03/102239 A2), among others each of which is incorporated herein by reference in its entirety.

In nucleic acid amplifications extent of amplification is extremely high, from a few copies billions of copies of the target nucleic acid can be synthesized. But problem with the nucleic acid amplification is generation of non-specific amplification products along with the specific amplification product. One frequently observed type of non-specific amplification product is a template independent amplification artifact “primer dimer”. Primer dimer is a double-Stranded amplification product whose length typically is close to the sum of the length of the two primers used for the amplification reaction. It forms when one primer is extended over the other primer. The resulting concatenation forms an undesired template which, because of its short length, is amplified more efficiently and competes with the specific target amplification product. Similarly, in case of use of a probe in additions to two amplification primers, similar undesired concatenated amplification artifact “primer dimer like” product is formed when one primer is extended over the probe and is typically of the length equal to the sum of the lengths of the two primers and the probe. These non-specific amplification products are formed more when copy number of the target nucleic acid is low, inhibitors are present in the sample and quality of the reagents, i.e., primers and probe is not good. It is very difficult to avoid formation of non-specific amplification products, can be reduced but elimination is impossible. Formation of these non-specific products brings down the specificity and sensitivity of the detection methods.

Further, in all these signal generation chemistries non-specific amplification products also equally generate signal along with target amplification product that affects the target detection sensitivity and specificity. To overcome this problem suppression of non-specific amplification product formation has been provided as the solution to the problem. And towards this hot start amplification, specifically hot start PCR amplification method has been employed as a solution. Many hot start methods with incremental improvement have been developed.

For example, use of heat sensitive materials like wax to separate or sequester reagents, U.S. Pat. No. 5,411,876, heat-reversible inhibition of the DNA polymerase by DNA polymerase-specific antibodies, as described in U.S. Pat. Nos. 5,338,671, 5,677,152, enzymatically degrading extension products formed prior to the start of the reaction using the methods described in U.S. Pat. No. 5,418,149, Chemically modifying primer or primers at their 3′ ends, U.S. Pat. No. 6,001,611, reversible chemical modification of the nucleic acid polymerase, which reverse or dissociate at an elevated temperature (U.S. Pat. No. 5,773,258 and Moretti et al, Biotechniques 25: 716, 1998) among others and incorporated by refence in entirety. A recent US patent application bearing the no US 2020/0370109 dated Nov. 26, 2020 discloses dual hot start solution which uses combination of two or more existing hot start mechanisms.

In of spite of all of the solutions a complete or very good solution to the non-specific amplification problem of nucleic acid amplification, which increases the sensitivity and specificity of nucleic acid target amplification, is still eluding. There is not a single process that has both good sensitivity and specificity; all existing process have almost same specificity and sensitivity (about 95% and 70-80% respectively). Non-specific amplification brings down the sensitivity and specificity. Other than hot-start PCR there has not been any other reasonable solution that comes closer to hot-start PCR. Hot start PCR allows prevention of non-specific product formation during reaction setting and the initial denaturation step. Thereafter, the polymerase becomes active at all temperatures in subsequent thermal cycling or amplification cycles and therefore formation of primer dimer or primer dimer like non-specific products during this cycling step cannot be avoided. Because of their small size, non-specific amplification products are relatively more efficient templates for amplification. Formation of the non-specific product is a stochastic phenomenon and if non-specific product forms at an early cycle of the amplification process it overtakes amplification of the target sequence resulting in failure of the target amplification or lowering of the yield of target amplification product and lower sensitivity of target detection. Hence there is still a need for a solution to this problem and an improved process for nucleic acid amplification for detection of target nucleic acids with higher specificity and sensitivity as discussed in embodiments of the present invention. As a result, this problem has been a technical limitation for the growth of PCR technology which has not been overcome in the last three decades. As a result, PCR and qPCR is in the process of losing out to digital droplet PCR (dPCR), which is a new technology and requires very expensive equipment. Hence there is a need for a solution or improvement to the above problem of PCR and other amplification processes in a cost-effective manner. Therefore, there is a need for further improvement or a better solution to the problem.

### SUMMARY

An aspect relates to a method for nucleic acid amplification for detection and/or quantification of target nucleic acid.

Another aspect of embodiments of the present invention is to provide a method for nucleic acid amplification with higher sensitivity and higher specificity and without any signal or insignificant signal from the primer dimer and primer dimer like non-specific nucleic acid amplification products..

Another aspect of embodiments of the present invention is to provide a method involving reduction or elimination of signal from primer-dimer and primer dimer like nonspecific amplification products thereby resolving the problem with a solution to the problems of conventional PCR or quantitative PCR (qPCR) and other nucleic acid amplification methods due to the interference of such non-specific signals.

Another object is to provide kit or kits for the amplification of target nucleic acids and detection and/or quantification of target nucleic acid, wherein the kits comprise either a labelled primer pair of embodiments of the invention labelled separately with a donor fluorophore or an acceptor fluorophore (radiative quencher)/non-radiative quencher, or a donor fluorophore labelled probe, an acceptor fluorophore (radiative quencher)/non-radiative quencher labelled primer and an unlabelled primer or two acceptor fluorophore (radiative quencher)/non-radiative quencher labelled primers of embodiments of the invention and embodiments of the present invention also can include one or more amplification reagents, e.g., a nucleic acid polymerase or ligase, nucleoside triphosphates, and suitable buffers without limitation.

Yet another aspect is to achieve absolute quantification of target nucleic acid in a sample using donor fluorophore and acceptor fluorophore (radiative quencher)/non-radiative quencher labelled primers or donor fluorophore labelled probe and acceptor fluorophore (radiative quencher)/non-radiative quencher labelled primer(s) of embodiments of the invention.

Thus, according to the basic aspect of embodiments of the present invention there is provided a method of nucleic acid target detection and/or quantification by nucleic acid amplification comprising: providing at least one target nucleic acid, at least one oligonucleotide non-extendable or extendable, labelled with at least one shining group/moiety adapted to shine extra upon hybridization or getting incorporated into a nucleic acid molecule;

providing at least one extendable oligonucleotide labelled with at least one converter or acceptor group/moiety adapted to convert the color of the shining group/moiety to a different color or thermalize it, is incorporated into an amplification product,

hybridizing or incorporating into a target nucleic acid amplification product the shining group/moiety of the shining group labelled oligonucleotide whereby the shining group/moiety shines extra with the extra shine being a measure of the target amplification,

and wherein the nucleic acid amplification is carried out such that any non-specific amplification does not generate any detectable signal under controlled attenuation by selectively controlling the removing only of the extra shine of the shining group in the non-specific amplification product by the converter or acceptor group/moiety by selective positioning of the signaling moiety on signaling oligonucleotide and selective positioning of the attenuator or acceptor moiety on attenuator or acceptor oligonucleotide for a particular signaling moiety and attenuator or acceptor moiety pair such that there is no net signal enhancement and no net signal from non-specific product, no net signal attenuation and no loss of target amplification signal.

Another aspect of embodiments of the present invention provide the method wherein, the extent of attenuation of signal in non-specific amplification product is equal or almost equal to the extent of enhancement of signal of the signaling moiety in non-specific amplification product so that there is no net signal enhancement and no net signal from non-specific product, no net signal attenuation and no loss of target amplification signal.

Yet another aspect of embodiments of the present invention is to provide the method wherein, the distance of the signaling moiety labeled base from 3′ end of the signaling moiety labelled oligonucleotide plus the distance of the attenuator or acceptor moiety labeled base from 3′ end of the receiver or acceptor moiety labelled oligonucleotide minus the distance of possible overlap between the 3′ ends of the signaling moiety labelled oligonucleotide and the attenuator or acceptor moiety labelled oligonucleotide comprises of the distance of separation between the signaling moiety and the attenuator or acceptor moiety labeled bases in non-specific amplification product that results in attenuation of signal equal or almost equal to the extent of enhancement of signal in the non-specific amplification product.

Yet another aspect of embodiments of the present invention provides the method wherein, the number of bases separating the signaling moiety labeled base from 3′ end of the signaling moiety labeled oligonucleotide plus the number of bases separating of the attenuator or acceptor moiety labeled base from 3′ end of the attenuator or acceptor moiety labelled oligonucleotide minus the number of possible base overlap between the 3′ ends of the signaling moiety labelled oligonucleotide and the attenuator or acceptor moiety labelled oligonucleotide comprises of the number of bases separating the signaling moiety and the attenuator or acceptor moiety labeled bases in non-specific amplification product that results in attenuation of signal equal or almost equal to the extent of enhancement of signal in the non-specific amplification product.

Yet another aspect of embodiments of the present invention provides the method wherein, the distance of the signaling moiety labeled base from 3′ end of the signaling moiety labelled oligonucleotide plus the distance of the attenuator or acceptor moiety labeled base from 3′ end of the receiver or acceptor moiety labelled oligonucleotide minus the distance of possible overlap between the 3′ ends of the signaling moiety labelled oligonucleotide and the attenuator or acceptor moiety labelled oligonucleotide comprises of the distance of separation between the signaling moiety and the attenuator or acceptor moiety labeled bases in non-specific amplification product that results in attenuation of signal equal or almost equal to the extent of enhancement of signal in the non-specific amplification product.

Another aspect of embodiments of the present invention provides the method wherein, the extent of signal enhancement and respective attenuation is selectively controlled such that for (i) signal enhancement of about 1-100% the respective attenuation of signal for balanced non-substantial signal from non-specific amplification product is maintained at about 1-50%; (ii) signal enhancement of about 20-80% the respective attenuation of signal for balanced non-substantial signal from non-specific amplification product is maintained at about 17-45% (iii) signal enhancement of about 30-70% the respective attenuation of signal for balanced non-substantial signal from non-specific amplification product is maintained at about 23-40%.

Yet another aspect of embodiments of the present invention provides the method wherein, the signaling moiety labeled oligonucleotide used is a probe for monitoring nucleic acid amplification which is hybridized to one strand of the target amplification product or the target and the attenuator or acceptor moiety labeled oligonucleotide used is one of the nucleic acid amplification primers which is annealed either to the same strand to which the probe hybridizes or the other strand of the target amplification product or target and wherein both amplification primers used are selectively labeled with attenuator or acceptor moiety.

Another aspect of embodiments of the present invention provides the method wherein, the signaling moiety is placed on signaling moiety labeled oligonucleotide either on its 3′ end or on any base up to 30 bases away from the 3′ end except the 5′ end and the attenuator or acceptor moiety is placed on attenuator or acceptor moiety labeled oligonucleotide on any base at least two bases away from the 3′ end or on any base up to 30 bases away from the 3′ end.

Still another aspect of embodiments of the present invention provides the method wherein, the signaling moiety labeled oligonucleotide and the attenuator or acceptor moiety labeled oligonucleotides used are two nucleic acid amplification primers that anneal separately to two strands of the target amplification product or the target nucleic acid and get extended by polymerase or polymerases.

A further aspect of embodiments of the present invention provides the method wherein, the signaling moiety is placed on signaling moiety labeled oligonucleotide either on any base at least two nucleotides away from the 3′ end or on any base up to 30 bases away from the 3′ end except the 5′ end and the attenuator or acceptor moiety is placed on attenuator or acceptor moiety labeled oligonucleotide on any base at least two nucleotides away from the 3′ end or on any base up to 30 bases away from the 3′ end.

Yet another aspect of embodiments of the present invention provides the method wherein the signaling moiety used is a donor fluorophore and the attenuator or acceptor moiety used is an acceptor fluorophore or a non-radiative acceptor or quencher moiety that accepts energy but do not emit any energy or electromagnetic radiation, signal is fluorescent signal and signal attenuation is quenching of fluorescence signal of the donor fluorophore and the donor fluorophore and acceptor moiety is an energy transfer pair.

Another aspect of embodiments of the present invention provides the method wherein, the distance of separation between the signaling moiety and the attenuator or acceptor moiety labeled bases in non-specific amplification product is selected such that would result in attenuation of signal equal or almost equal to the extent of enhancement of signal in the non-specific amplification product is respectively between 2R₀ distance and R₀ distance where 3.7 angstrom is inter-base distance and R₀ is the Forster radius of the signaling moiety and the acceptor moiety pair, more specifically, the donor fluorophore and acceptor fluorophore or non-radiative acceptor moiety pair (Forster Radius R₀ value range 22 Å to 75 Å).

A further aspect of embodiments of the present invention provides the method wherein, the distance of separation between the signaling moiety and the attenuator or acceptor moiety labeled bases in non-specific amplification product is selected such that result in attenuation of signal equal or almost equal to the extent of enhancement of signal in the non-specific amplification product which is selectively respectively between 1.3025 R₀ distance and 1.034 R₀ distance where 3.7 angstrom is inter-base distance and R₀ is the Forster radius of the signaling moiety and the acceptor moiety pair, more specifically., the donor fluorophore and acceptor fluorophore or non-radiative acceptor moiety pair (Forster Radius R₀ value range 22 Å to 75 Å).

Yet another aspect of embodiments of the present invention provides the method wherein, the distance of separation between the signaling moiety and the attenuator or acceptor moiety labeled bases in non-specific amplification product used is such that result in attenuation of signal equal or almost equal to the extent of enhancement of signal in the non-specific amplification product which is respectively between 1.224 R₀ distance and 1.0699 R₀ distance where 3.7 angstrom is inter-base distance and R₀ is the Forster radius of the signaling moiety and the acceptor moiety pair, i.e., the donor fluorophore and acceptor fluorophore or non-radiative acceptor moiety pair (Forster Radius R₀ value range 22 Å to 75 Å).

Still another aspect of embodiments of the present invention provides the method wherein, the number of bases separating the signaling moiety and the attenuator or acceptor moiety labeled bases in non-specific amplification product is selected such that result in attenuation of signal equal or almost equal to the extent of enhancement of signal in the non-specific amplification product equivalent to the distance between 1.3025 (R₀) and 1.034 (R₀) is about (1.3025R₀/3.7) bases to (1.034R₀/3.7) bases where 3.7 angstrom is interbase distance and R₀ is the Forster radius for the donor fluorophore and acceptor moiety energy transfer pair expressed in angstrom and is equivalent to about 6.1 to 26.4 bases for the entire range of fluorophore moiety and acceptor moiety energy transfer pairs (Forster Radius R₀ value range 22 Å to 75 Å).

Still another aspect of embodiments of the present invention provides the method wherein, the number of bases separating the signaling moiety and the attenuator or acceptor moiety labeled bases in non-specific amplification product is selected such that result in attenuation of signal equal or almost equal to the extent of enhancement of signal in the non-specific amplification product equivalent to the distance between 1.3025 (R₀) and 1.034 (R₀) is about (1.3025R₀/3.7) bases to (1.034R₀/3.7) bases where 3.7 angstrom is inter-base distance and R₀ is the Forster radius for the donor fluorophore and acceptor moiety energy transfer pair expressed in angstrom and is equivalent to about 6.1 to 26.4 bases for the entire range of fluorophore moiety and acceptor moiety energy transfer pairs (Forster Radius R₀ value range 22 Å to 75 Å).

Another aspect of embodiments of the present invention provides the method wherein, the number of bases separating the signaling moiety and the attenuator or acceptor moiety labeled bases in non-specific amplification product is selected such that would result in attenuation of signal equal or almost equal to the extent of enhancement of signal in the non-specific amplification product equivalent to the distance between 1.224 (R₀) and 1.0699 (R₀) is about (1.224R₀/3.7) bases to (1.0699 R₀/3.7) bases where 3.7 angstrom is inter-base distance and R₀ is the Forster radius for the donor fluorophore and acceptor moiety energy transfer pair expressed in angstrom and is equivalent to about 6.4 to 24.8 bases for the entire range of fluorophore moiety and acceptor moiety energy transfer pairs (Forster Radius R₀ value range 22 Å to 75 Å).

Yet another aspect of embodiments of the present invention provides the method wherein, the signaling moiety labeled oligonucleotide and attenuator or acceptor moiety labeled oligonucleotide used are linear.

Still another aspect of embodiments of the present invention provides the method wherein additional labeling moiety biotin or the like is placed at 3′ and/5′ end of the labeled probe.

Another aspect of embodiments of the present invention provides the method wherein, the signaling moiety or donor fluorophore labelled probe of claim 6 or the signaling moiety or donor fluorophore moiety labelled primer of claims 8 are additionally provided at 5′ end with acceptor fluorophore or a quencher or a five to eight bases sequence sufficiently complementary to the sequence of the labelled primer or probe in the vicinity of the fluorophore labelled base to form a stem structure and are provided with or without a quencher and with or without intervening spacer between the fluorophore labelled primer or probe and the five to eight bases sequence.

Still another aspect of embodiments of the present invention provides the method wherein, the number of bases separating the signaling moiety or donor fluorophore moiety labeled base and the 3′ end of the donor fluorophore labelled primer or probe plus the number of bases separating the acceptor or quencher moiety labelled base and the 3′ end of the acceptor or quencher moiety labelled primer is 6-40 bases for the entire range of donor fluorophore and acceptor or quencher moiety energy transfer pairs.

A further aspect of embodiments of the present invention provides the method wherein, the number of bases separating the signaling moiety or fluorophore labeled base and the 3′ end of the fluorophore labelled primer or probe plus the number of bases separating the acceptor or quencher moiety labelled base and the 3′ end of the acceptor or quencher moiety labelled primer is 10-30 bases for the entire range of donor fluorophore and acceptor or quencher moiety energy transfer pairs.

Still further aspect of embodiments of the present invention provides the method wherein, more than one signaling moiety or donor fluorophore are placed on the signaling moiety labeled probe or primer and more than one acceptor moiety or quencher are placed on the acceptor moiety labeled primer or primers.

Another aspect of embodiments of the present invention provides the method wherein a first primer pair amplifies a first segment of the target nucleic acid and a second primer pair amplifies a second segment of the first segment in nested nucleic acid amplification, the second primer pair is the signaling or donor fluorophore moiety labelled and the acceptor or quencher moiety labelled libra primer pair including two nucleic acid amplification primers selected from second primer pair are labeled with acceptor or quencher moiety.

A further aspect of embodiments of the present invention provides the method wherein, the primers of the first primer pair are additionally provided suitably labelled with acceptor or quencher moiety.

Yet another aspect of embodiments of the present invention provides the method wherein a first primer and a second primer together amplify a first segment of a target nucleic acid and a third primer in association with the first primer amplifies a second segment of the first segment in a semi-nested nucleic acid amplification, the first and the third primers are the signaling or fluorophore moiety labelled and the acceptor or quencher moiety labelled libra primer pair wherein the second primer is also provided additionally labelled with acceptor fluorophore or quencher.

A further aspect of embodiments of the present invention provides the method wherein a first primer pair amplifies a first segment of the target nucleic acid, a second primer pair amplifies a second segment of the first segment in nested nucleic acid amplification and a probe is used where the probe hybridizes to the second segment, the probe and the second primer pair are respectively the signaling or donor fluorophore moiety labelled probe and the acceptor or quencher moiety labelled primer(s); wherein the first primer pair is also additionally provided suitably labelled with acceptor or quencher moieties.

Yet another aspect of embodiments of the present invention provides the method wherein a first primer and a second primer together amplify a first segment of the target nucleic acid, a third primer with the first primer amplify a second segment of the first segment in semi-nested nucleic acid amplification and a probe that hybridizes to the second segment is used, the probe and the first and/or third primers are respectively the signaling or donor fluorophore moiety labelled probe and the acceptor or quencher moiety labelled primer(s).

A further aspect of embodiments of the present invention provides the method wherein, a semi-synthetic target nucleic acid sequence is generated by appending a first or a first and second non-target sequences at one or both ends of a target nucleic acid sequence by any known methods in the art (there is a lot of them, any person of ordinary skill is aware of these methods) and amplification of the target nucleic acid is driven either by one target specific primer and the first non-target sequence specific primer or by the first and second non-target sequence specific primers respectively, where the amplification primers are the signaling or donor fluorophore moiety and acceptor or quencher moiety labelled primers of claim 8 or embodiments of the invention including selectively SEQ ID 21 & 22 and wherein also the first and the second non-target sequences are appended to a target sequence by tail PCR, where the first and second non-target sequences are appended at 5′ ends of two PCR primers.

A further aspect of embodiments of the present invention provides the method wherein, additionally provided a probe where the probe and the primers are labelled primer(s) and probe of the invention wherein, the signaling moiety or donor fluorophore labelled probe.

Yet another aspect of embodiments of the present invention provides the method wherein the signaling moiety or donor fluorophore labelled primer or probe including selectively SEQ ID NO.:22 is additionally provided at 5′ end either with a quencher/acceptor fluorophore or appended with a five to eight bases sequence that hybridizes to the bases near the donor fluorophore labelled base of the donor fluorophore labelled primer or probe forming hair pin and is with or without a quencher at the 5′ end and is linked with or without a linker.

A further aspect of embodiments of the present invention provides the method wherein, the extent of signal enhancement or fluorescence enhancement of the donor fluorophore on incorporation of the signaling or fluorophore moiety labelled primer into the target amplification product or hybridization of the signaling or fluorophore moiety labelled probe to the target amplification product is 4-8-fold and extent of signal attenuation or fluorescence quenching in non-specific amplification product for non-substantial signal from such products is 75-87% and the separation of the signaling or fluorophore moiety and the acceptor or quencher moiety is 0.8327 R₀ to 0.7284 R₀ which is equivalent to 0.8327 (R₀/3.7) bases to 0.7284 (R₀/3.7) bases where 3.7 angstrom is inter-base distance and R₀ is the Forster radius for the fluorophore and acceptor moiety energy transfer pair in angstrom unit and is equivalent to about 4 to 16 bases for the entire range of fluorophore moiety and acceptor moiety energy transfer pairs (Forster Radius R₀ value range 22 Å to 75 Å).

Another aspect of embodiments of the present invention provides the method wherein, the number of bases separating the signaling moiety or fluorophore labeled base and the 3′ end of the fluorophore labelled primer or probe plus the number of bases separating the acceptor or quencher moiety labelled base and the 3′ end of the acceptor or quencher moiety labelled primer is 4-16 bases.

A further aspect of embodiments of the present invention provides the method 32 wherein, a first signaling or first fluorophore moiety labelled first primer, an acceptor or quencher moiety labelled second primer and a second signaling or second fluorophore moiety labelled probe are used in nucleic acid amplification, where the first signaling or first fluorophore moiety generates a first signal on target amplification and the second signaling or second fluorophore moiety of the probe generates a second fluorescent signal on hybridization of the probe to the target amplification product, the first signaling or first fluorophore labelled first primer and the acceptor or quencher moiety labelled second primer are the signaling or fluorophore moiety and acceptor or quencher moiety labelled primer pair of embodiments of the invention and the first signaling or fluorophore moiety labelled first primer, the acceptor or quencher moiety labelled second primer and the second signaling or second fluorophore moiety labelled probe are the labelled primers and probe of embodiments of the invention and the first signaling or first fluorophore moiety is an acceptor fluorophore or quencher for the second signaling or second fluorophore moiety or vice versa.

A further aspect of embodiments of the present invention provides the method wherein, a donor fluorophore labelled primer and an acceptor fluorophore labelled primer are used to amplify a target nucleic acid sequence where the donor fluorophore and the acceptor fluorophore are placed at least 2 nucleotides away from the 3′ ends of the primers and the donor fluorophore and the acceptor fluorophore are separated by 5 to 30 bases in the target amplification product so that a FRET signal, more specifically the emission from acceptor fluorophore on excitation of the donor fluorophore is generated on target amplification.

Another aspect of embodiments of the present invention provides the method wherein, one of the two amplification primers is provided labelled with an acceptor fluorophore and a donor fluorophore labelled probe that hybridizes to the target sequence is used instead for target nucleic acid amplification, where the acceptor fluorophore is placed at least 2 nucleotides away from the 3′ end of the labelled primer and the donor fluorophore labelled probe is labelled with the donor fluorophore at its 3′ end or on a base away from the 3′ end except the 5′ end base, the donor fluorophore and the acceptor fluorophore are separated by 5 or more bases when the probe hybridizes to the target amplification product so that a FRET signal more specifically, emission from the acceptor fluorophore on excitation of the donor fluorophore is generated on target amplification.

Still further aspect of embodiments of the present invention provides the method wherein, the probe is provided labelled with the acceptor fluorophore and the labelled primer is provided labelled with the donor fluorophore instead.

Yet another aspect of embodiments of the present invention provides the method wherein the donor fluorophore and the acceptor fluorophore are separated by 10-20 bases in target amplification.

A further aspect of embodiments of the present invention provides the method the donor fluorophore and the acceptor fluorophore are separated by 14-20 bases in the target amplification product.

Another aspect of embodiments of the present invention provides the method wherein, the distance or the number of bases separating the donor fluorophore labelled base from the 3′ end of the donor fluorophore labelled primer or probe plus the distance or the number of bases separating the acceptor fluorophore labelled base from the 3′ end of the acceptor fluorophore labelled primer or probe, minus the distance of overlap or no of bases overlap between the 3′ ends of the donor fluorophore labelled primer or probe and the acceptor fluorophore labelled primer or probe respectively is equal to the distance or equivalent base separation for static quenching between the donor fluorophore and the acceptor fluorophore for a non-substantial signal from the non-specific amplification products.

A further aspect of embodiments of the present invention provides the method wherein, the distance of static quenching is plus-minus 3 or plus minus 2 or plus minus 1 or 0 or the labelled bases are placed opposite to each other.

Yet another aspect of embodiments of the present invention provides the method wherein, a promoter sequence is appended to a target nucleic acid sequence, a single or double stranded DNA or RNA, using a first oligonucleotide primer carrying at its 3′ end a target specific sequence or a poly Thymidine sequence or a poly Thymidine sequence with one or more non-thymine bases and a promoter sequence at the 5′ end or using a double stranded adaptor with a 5′ end protrusion of a few bases for attaching the adaptor to the target sequence and the promoter sequence at the 5′ end of the adaptor or with a 3′ end protrusion of a few bases for attaching the adaptor to the target sequence and the promoter sequence at the 5′ end of the adaptor or using a first oligonucleotide primer with a stretch of target sequence and a promoter sequence at its 5′ end;

a linear amplification of the target sequence is carried out by sequential polymerase extension using a second target specific primer and the promoter sequence carrying first oligonucleotide primer using a DNA polymerase or Reverse transcriptase and deoxy nucleoside triphosphates in combination with RNA transcription using an RNA polymerase and ribonucleoside triphosphates, where the first promoter sequence carrying primer and the second target specific primer are the donor fluorophore and the quencher / acceptor fluorophore labelled primer pairs of embodiments of the invention claims 8-10 and 17 wherein the signaling moiety labeled oligonucleotide and the attenuator or acceptor moiety labeled oligonucleotides used are two nucleic acid amplification primers that anneal separately to two strands of the target amplification product or the target nucleic acid and get extended by polymerase or polymerases.(claim 8); the signaling moiety is placed on signaling moiety labeled oligonucleotide either on any base at least two nucleotides away from the 3′ end or on any base up to 30 bases away from the 3′ end except the 5′ end and the attenuator or acceptor moiety is placed on attenuator or acceptor moiety labeled oligonucleotide on any base at least two nucleotides away from the 3′ end or on any base up to 30 bases away from the 3′ end(claim 9); wherein the signaling moiety used is a donor fluorophore and the attenuator or acceptor moiety used is an acceptor fluorophore or a non-radiative acceptor or quencher moiety that accepts energy but do not emit any energy or electromagnetic radiation, (claim 10) and the signaling moiety labeled oligonucleotide and attenuator or acceptor moiety labeled oligonucleotide used are linear (Claim 17)

Another aspect of embodiments of the present invention provides the method wherein, either or both of the promoter sequence carrying first primer and the second primer are labelled with quencher or acceptor fluorophore and a target specific probe labelled with a donor fluorophore are used and the labelled probe and primer(s) are the donor fluorophore labelled probe and acceptor fluorophore or quencher labelled primer(s)of embodiments of the invention having the signaling moiety or donor fluorophore labelled probe of claim 6; wherein, the signaling moiety is placed on signaling moiety labeled oligonucleotide either on its 3′ end or on any base up to 30 bases away from the 3′ end except the 5′ end and the attenuator or acceptor moiety is placed on attenuator or acceptor moiety labeled oligonucleotide on any base at least two bases away from the 3′ end or on any base up to 30 bases away from the 3′ end(claim 7); wherein the signaling moiety used is a donor fluorophore and the attenuator or acceptor moiety used is an acceptor fluorophore or a non-radiative acceptor or quencher moiety that accepts energy but do not emit any energy or electromagnetic radiation (Claim 10); the signaling moiety labeled oligonucleotide and attenuator or acceptor moiety labeled oligonucleotide used are linear (Claim 17).

A further aspect of embodiments of the present invention provides the method wherein, a quencher is also attached at 5′ the end of the donor fluorophore labelled primer or probe.

Still further aspect of embodiments of the present invention provides the method wherein, the nucleic acid amplification reactions comprises Polymerase Chain Reactions (PCR), where the polymerase chain reactions (PCR) are without limitation Polymerase Chain Reaction (PCR), Reverse Transcription Polymerase Chain Reaction (RT-PCR), Allelic or Allele specific Polymerase Chain Reaction (Allelic PCR), Allelic RT-PCR, Tail PCR, Droplet PCR, Emulsion PCR, Digital PCR, Asymmetric PCR (where one of the two primers are used in a very low concentration in comparison to other primer and single stranded target amplification product is generated), Nested PCR, Semi-Nested PCR, methylation status PCR, in-situ PCR and the size of the target amplification product is 35 to 400 base pairs for example, 50 to 250 base pairs.

A further aspect of embodiments of the present invention provides the method wherein, the nucleic acid amplification reaction comprises Isothermal Nucleic Acid Amplifications Reactions including but not limited to Loop Mediated Isothermal Nucleic acid Amplification Reactions (LAMP), Recombinase Polymerase Amplification Reactions (RPA), Helicase Polymerase Amplification Reactions (HPA), Nucleic acid sequence based amplification (NASBA) where loop primers used in LAMP facilitate DNA strand separation, strand separating enzyme like Recombinase, Helicase, Gyrase, Topoisomerase are used in RPA and HPA for denaturation or strand separation in association with single strand binding (SSB) proteins and the size of the target amplification product is 75 to 1000 base pairs, for example, 100 to 250 base pairs.

A further aspect of embodiments of the present invention provides the method wherein absolute quantification of target nucleic acid is carried out using labelled primer—probe pair wherein the signaling moiety labeled oligonucleotide used is a probe for monitoring nucleic acid amplification which is hybridized to one strand of the target amplification product or the target and the attenuator or acceptor moiety labeled oligonucleotide used is one of the nucleic acid amplification primers (of claim 6) Wherein, the signaling moiety is placed on signaling moiety labeled oligonucleotide either on its 3′ end or on any base up to 30 bases away from the 3′ end except the 5′ end and the attenuator or acceptor moiety is placed on attenuator or acceptor moiety labeled oligonucleotide on any base at least two bases away from the 3′ end or on any base up to 30 bases away from the 3′ end.(of claim 7);

wherein the signaling moiety used is a donor fluorophore and the attenuator or acceptor moiety used is an acceptor fluorophore or a non-radiative acceptor or quencher moiety that accepts energy but do not emit any energy or electromagnetic radiation, signal is fluorescent signal and signal attenuation is quenching of fluorescence signal of the donor fluorophore and the donor fluorophore and acceptor moiety is an energy transfer pair (claim 10)

and labelled primer pair including SEQ ID No.: 21 & 22 selectively and a kit or kits for the same wherein the kit or kits comprise in one or more containers at least one the donor fluorophore labelled oligonucleotide probe and acceptor fluorophore or quencher labelled oligonucleotide primer(s) or at least a donor fluorophore labelled oligonucleotide primer and acceptor fluorophore or quencher labelled oligonucleotide primer wherein, the signaling moiety labeled oligonucleotide and the attenuator or acceptor moiety labeled oligonucleotides used are two nucleic acid amplification primers that anneal separately to two strands of the target amplification product or the target nucleic acid and get extended by polymerase or polymerases

the kit or kits may additionally contain reaction buffer, plurality of deoxy nucleoside triphosphates, polymerase enzyme or enzymes, positive control template and corresponding labelled primer pair for amplification and additional components.

Another aspect of embodiments of the present invention provide the method wherein, the oligonucleotides are selectively of 10-50 bases long, for example 15-35 bases long or 20-30 bases long, complementary to the target sequence, having the ability to hybridize or anneal and prime nucleic acid synthesis on the target is not lost and carry one or more modified bases, or modified sugar moiety/moieties, or one or more base analogues.

A further aspect of embodiments of the present invention provide the method wherein, a positive control template and positive control template specific labelled primer pair or labelled primer and probe pair of claims 6-17, 28, 29, 34, 35, 46 are additionally provided in the amplification reaction.

Yet another aspect of embodiments of the present invention provide the method wherein, multiple donor fluorophore labelled and acceptor fluorophore/quencher labelled primer pairs or multiple donor fluorophore labelled probe and acceptor fluorophore/quencher labelled primer pairs of embodiments of the invention claims 6-17, 28-29, 34-35, 46 are used in a multiplexing reaction for simultaneous detection and/or quantification of multiple target sequences.

A further aspect of embodiments of the present invention provide the method wherein, one or multiple or large array of donor fluorophore labelled primers or donor fluorophore labelled probes are attached or covalently linked or tethered through multi-carbon atom organic linker or polyethylene glycol or hybrid linker or polythymidine oligonucleotide with or without additional organic linker of sufficient length to a solid surface like glass or glass wafer or plastic like polystyrene, polyethylene, polypropylene or dextran, cellulose, nylon, transparent or translucent, microfluidic channels is used for the detection of a single or multiple or large number of nucleic acid targets in a single amplification reaction.

Another aspect of embodiments of the present invention provide the method wherein the nucleic acid amplification reaction comprise any known nucleic acid amplification reaction, for example, polymerase chain reaction comprise at least the steps of adding at least one Polymerase enzyme, reaction buffer, deoxy nucleoside triphosphates in addition to the effective amounts of amplification primers or effective amounts of labelled primer and labelled probe or labelled primers to the sample, cycling the sample, between at least a denaturation step, an annealing step, an extension step or a single combined annealing and extension step, or in an isothermal reaction step, exciting the reaction mixture with the donor fluorophore exciting light or radiation, measuring the emission of the donor fluorophore or the acceptor fluorophore.

A further aspect of embodiments of the present invention provide the method wherein the donor fluorophore, acceptor fluorophore and quenchers are selected from the group comprising double stranded DNA intercalating dyes including but not limited to intercalating dyes Ethidium bromide, SYBR Green 1™ Picogreen™, YOPRO 1™, SYTO 9™, Acridine Orange, asymmetric cyanine dyes, that results in fluorescence enhancement on intercalation to double stranded DNA and the dyes Fluorescein, 5-Carboxyfluorescein (5-FAM), 6-Carboxyfluorescein (6-FAM), 6-FAM (Azide), 2′7′-dimethoxy—4′5-6—carboxyfluorescein (JOE), 5-(4,6—dichlorotriazin-2 yl) Aminofluorescein (DTAF), Fluorescein isothiocyanate, HEX (Hexachlorofluorescein), TET (Tetrachlorofluorescein), VIC (Victoria Blue), MAX VIC with spectral profile nearly identical to VIC, SUN™ A VIC® (ThermoFisher Scientific) equivalent, TYE™ 563, TYE 665, TYE 705, NED, fluorescamine, Pyrene, Pyrene butyrate, succimidyl 1 Pyrene butyrate, Rhodamine (Rhod), Rhodamine 123, Rhodamine B, Sulforhodamine, 6-carboxyrhodamine (R6G), 6-carboxy-Xrhodamine (ROX), Sulforhodamine b, Sulforhodamine 101, Sulphonyl chloride derivative of sulforhodamine 101 (Texas Red), Texas Red®-X, SIMA dye™, Texas Red®-X, TEX 615 N′, N′,N′,N′-Tetramethyl—6—carboxyrhodamine (TAMRA), TAMRA™ (Azide), Rhodamine Green™-X, Rhodamine Red™, Tetramethyl rhodamine, Tetramethyl rhodamine isothiocyanate (TRITC), Terbium chelates, Europium chelates, Quantum dots, graphene quantum dots, 5-(2′-aminoethyl) aminonapthylamide-3,5disulfonate(Lucifer Yellow vs), 7-amino-4-methylcoumarin (amc, coumarin 120), 7-amino-4-trifluoromethylcoumarin (coumarin 151), Cyanine dyes (sulphonate or non-sulphonated) including but not limited to Cyanine 2, Cyanine 3, Cyanine 3.5, Cyanine 5, Cyanine 5.5, Cyanine 7, [5-dimethylamino] Napthalene-1-sulfonyl chloride (DNS, Dancyl chloride), 4-(4′—dimethylaminopheylazo) benzoic acid (DABCYL), DABSYL, 4-dimethylaminopheylazophenyl)-4′-isithiocyanate (DABITC), IAEDANS (5-((((2-iodoacetyl) amino) Ethyl) amino) naphthalene sulfonic acid), EDANS, QST 7, QSY9, QSY 21, QSY 35 (QSY dyes are diaryl-rhodamine derivatives), BIODIPY Dyes including but not limited to BIODIPY FL, Alexa fluor dyes including but not limited to Alexa fluor 350, 488, 546, 555, 568, 594, 647, 660, 750 dyes, ATTO dyes including but not limited to ATTO™ 488 (NHS Ester), ATTO™ 532, ATTO™ 550, ATTO™ 565, ATTO™ 590, ATTO™ 633, ATTO™ 647N, ATTO™ Rhol01, ATTO™ 647N (NHS Ester), Yakima Yellow, MGB Dyes (major or minor groove binding dyes), LI-COR IRDyes® IRDye® 700, IRDye® 800, IRDye® 800CW, Lightcycler® 640, Dy 750, non-radiative quenchers including but not limited to Nanogold Particle, Blackhole quencher 0, Blackhole quencher 1, Blackhole quencher 2, Blackhole quencher 3, Eclipse Quenchers™, Dark Quenchers™, IDT quenchers Iowa Black™ RQ, Iowa Black FQ, ZEN™, TAO and/or ZEN®, Nano Particle Quenchers, Single Strand Binding Protein.

Yet another aspect of embodiments of the present invention provide the method wherein the Polymerase enzyme or enzymes used for nucleic acid amplification reactions are an enzyme that is a DNA Polymerase with or without strand displacement activity or template independent primer or base extension activity, exonuclease activity or a Reverse Transcriptase or a Polymerase with both Reverse Transcriptase and DNA Polymerase activity or a RNA Polymerase or a RNA polymerase and DNA polymerase, natural or modified or chimeric, where the polymerases can be thermostable, ambient temperature or below ambient temperature active enzyme, hot start polymerase where the polymerase become active after it is heated at an elevated temperature, for example, primer annealing temperature.

A further aspect of embodiments of the present invention provide the method used to detect a nucleic acid (methylated or unmethylated) or a non-nucleic acid target where a first binding moiety with very high affinity for nucleic acid or non-nucleic acid target is used to capture the nucleic acid or the non-nucleic acid target and a second binding moiety that can be the same first binding moiety or a different binding moiety with very high affinity for the nucleic acid or non-nucleic acid target is used to bind to the captured nucleic acid or the non-nucleic acid target or a third binding moiety that binds to the second binding moiety with very high affinity is used, where the second or the third binding moiety is provided appended with a synthetic or natural nucleic acid target molecule and the bound second or third binding moiety is detected and quantified after washing out the unbound nucleic acid appended second or third binding moiety by nucleic acid amplification using donor fluorophore and acceptor fluorophore/quencher labelled primer pair or probe and primer pair including selectively any of claims 6-17, 28-29, 34-35, 46, 54;

the binding moieties are selected, for example, from binding pairs antigen—antibody, protein-anti-protein antibody, antibody—antibody, antibody-anti-IgG antibody, first antibody—second antibody, Protein A-antibody, Protein G-antibody, biotin—avidin, biotin—streptavidin, lectin—sugar, nucleic acid—nucleic acid, protein-nucleic acid, peptide nucleic acid, aptamer—aptamer, aptamer—nucleic acid, aptamer-protein, hapten—anti-hapten antibody where the haptens are the small molecules including but not limited to the fluorescent dyes, bromo-d-UTP, aflatoxins and other mycotoxins, peptides, sugars.

Another aspect of embodiments of the present invention provide the method wherein, a method for detection and/or quantification of a large number m-RNAs or c-DNAs comprising providing first amplification primers specific for each m-RNA or c-DNA (DNA complementary of mRNA) and providing as second amplification primer a common primer (common for all m-RNAs or c-DNAs in the sample) selected from a sequence appended to the m-RNAs or c-DNAs and additionally probes specific for each m-RNA or c-DNA where the first amplification primers, the second common amplification primer and probes are primers and probes including selectively 6-17, 28-29, 34-35, 46.

A further aspect of embodiments of the present invention provide the method wherein, the donor fluorophore labelled probe is provided attached or linked to one of the primers through a non-nucleotide organic linker, hexamethylene, hexapolyethylene glycol or chimera or longer length of them and the probe hybridize to the nascent nucleic strand generated through extension of the linked primer.

In another aspect embodiments of the present invention provide a kit for carrying out a nucleic acid amplification reaction comprising in one or more containers at least a donor fluorophore labelled oligonucleotide probe and acceptor fluorophore or quencher labelled oligonucleotide primer(s) including selectively of claims 6, 10-17, 26-33, 35,42, 49-50, 54-56 including selectively
(a) at least a donor fluorophore labelled oligonucleotide primer and an acceptor fluorophore or quencher labelled oligonucleotide primers claims 8-17, 28, 34, 41, 43, 48-50, 54-56;
(b) at least donor fluorophore labelled oligonucleotide primer or primers and acceptor fluorophore or quencher labelled oligonucleotide primer or primers of including selectively of claims 28, 43, 48 & 50 and SEQ ID 21-22;
(c) at least donor fluorophore labelled oligonucleotide probe or probes and acceptor fluorophore or quencher labelled oligonucleotide primer or primers including selectively of claims 19, 23, 24-25, 29, 35, 48-50;
(d) at least donor fluorophore and acceptor fluorophore/quencher labelled promoter sequence carrying first primer and target specific second primer including selectively of claims 41,
(e) at least a positive control template and positive control template specific donor fluorophore and acceptor fluorophore/quencher labelled primer pair or primer and probe including selectively of claims 6-17, 24 -35, 41-43, 48-50, 54-56;
(f) labelled primer pair” wherein the donor fluorophore labelled probe is provided attached or linked to the acceptor fluorophore or quencher moiety labelled primer through a non-nucleotide organic linker, hexamethylene, hexapolyethylene glycol or chimera or longer length of them and the probe hybridize to the nascent nucleic strand generated through extension of the linked primer.

In another aspect embodiments of the present invention provide the kit including reaction buffer, plurality of deoxy nucleoside triphosphates, polymerase enzyme or enzyme, positive control template and positive template respective labelled primer pair.

Yet another aspect of embodiments of the present invention provide wherein, the target nucleic acid is purified or partially purified or un-purified nucleic acid is selected from natural or synthetic or semi-synthetic single or double stranded DNA or RNA, single or double stranded c-DNA, genomic DNA, methylated DNA, mitochondrial DNA, exosome DNA, plasmid DNA, ribosomal RNA (rRNA) transfer RNA (tRNA), messenger RNA (m-RNA),small RNA, including without limitation, micro-RNA, sRNA, stRNA, snoRNA, ncRNA, DNA from stem cell including very small embryonic like stem cells, viral DNA or RNA or cancer cell DNA from any source including but not limited to body fluids, biopsy samples, tumor, puss, saliva, faeces, cancer stem cell and synthetic or semisynthetic DNA or RNA, single or double stranded generated by appending one or two non-target synthetic sequences to the ends of the target nucleic acid. Further, a target nucleic acid need not constitute the entire nucleic acid molecule and also a genomic sequence of infectious agents, mutation (single base change or deletion or insertion of a few bases or long sequences) of genomic sequence or genomic sequence of human bacteria, yeast, fungi, plant, animal, human, parasites and their viruses and any other organism, live or dead, the presence or absence of which or mutation (single base change or deletion or insertion of a few bases or long sequences) of which is implicated to the presence of disease or disorder or susceptibility to infection or disease or disorder or suitability to a disease treatment, prenatal diagnosis, genetic trait, genotype, allele type, SNP detection, cell type, tissue type, species or strain type, cancer type or sub-type, cancer detection, disease typing or sub-typing, expressed gene.

### BRIEF DESCRIPTION

Some of the embodiments will be described in detail, with references to the following Figures, wherein like designations denote like members, wherein:

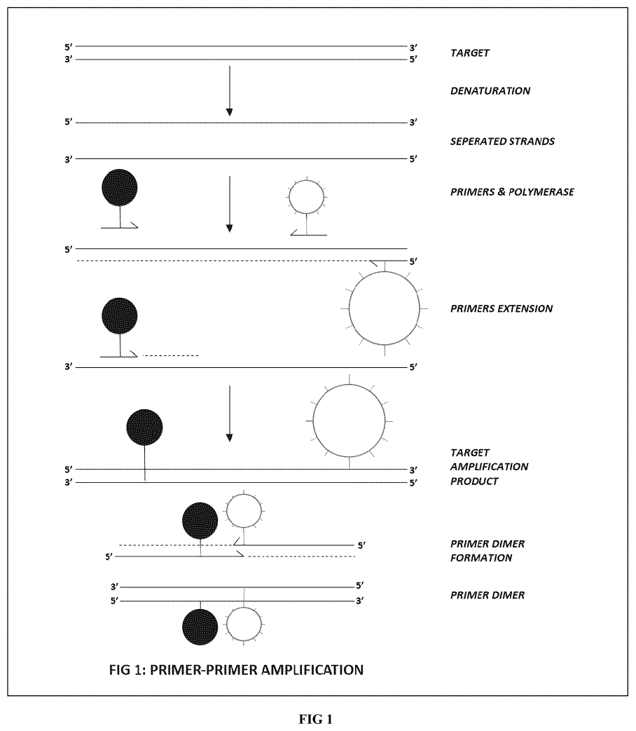

FIG. 1 shows a schematic representation of PCR amplification using an internal donor fluorophore labelled primer and an internal acceptor fluorophore/quencher labelled primer. Donor fluorophore is excited with donor specific excitation light and donor fluorophore emission is measured. On target amplification there is enhancement of donor fluorophore fluorescence and on formation of non-specific primer dimer there is simultaneous enhancement and quenching of donor fluorophore fluorescence.

When enhancement and quenching of donor fluorophore fluorescence is balanced there is no net fluorescence or net quenching of donor fluorophore which results in no signal from donor fluorophore and hence no signal from primer dimer;

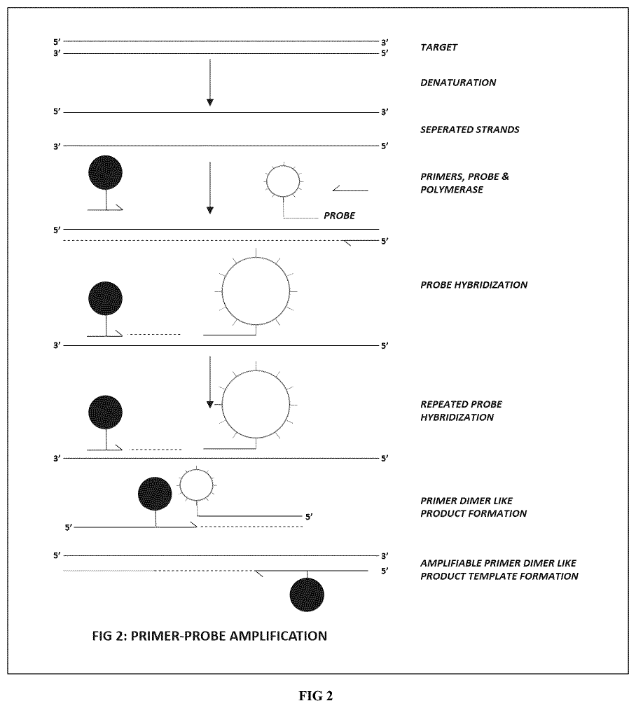

FIG. 2 shows a schematic representation of PCR amplification using an internal acceptor fluorophore/quencher labelled primer and a donor fluorophore labelled probe. Donor fluorophore is excited with donor fluorophore specific excitation light and donor fluorophore emission is measured. On target amplification and hybridization of the probe there is enhancement of donor fluorophore fluorescence and on formation of non-specific primer dimer like product and hybridization of donor fluorophore labelled probe, there is simultaneous enhancement and quenching of donor fluorophore fluorescence.

When enhancement and quenching of donor fluorophore fluorescence is balanced there is no net fluorescence or net quenching of donor fluorophore which results in no signal from donor fluorophore and hence no signal from primer dimer like non-specific product.

Further, the primer dimer like non-specific product initially formed gets extended against the strand complementary to the strand to which the probe hybridizes and generate an amplifiable primer dimer like non-specific product.

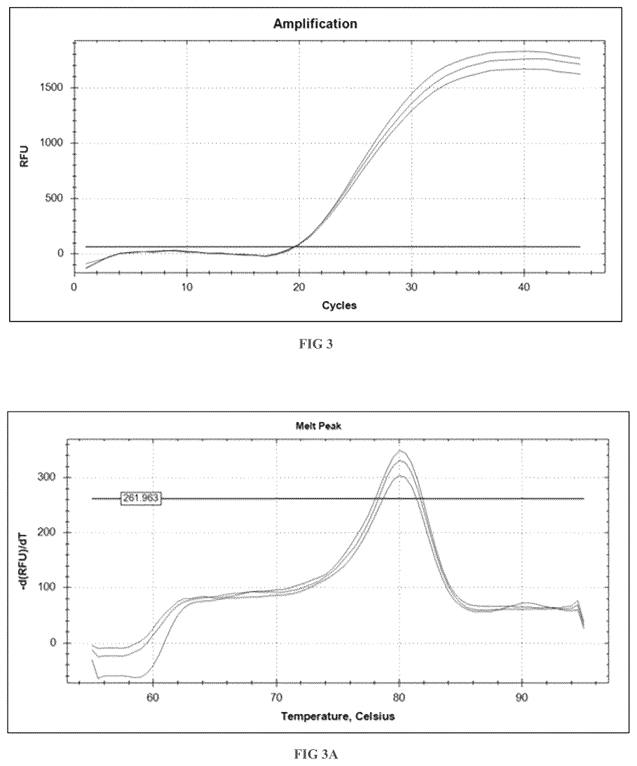

FIG. 3 shows amplification curves for the samples (1 ng DNA) and primer sequences SEQ ID NO.: 8 and 10;

FIG. 3A shows respective melt curves;

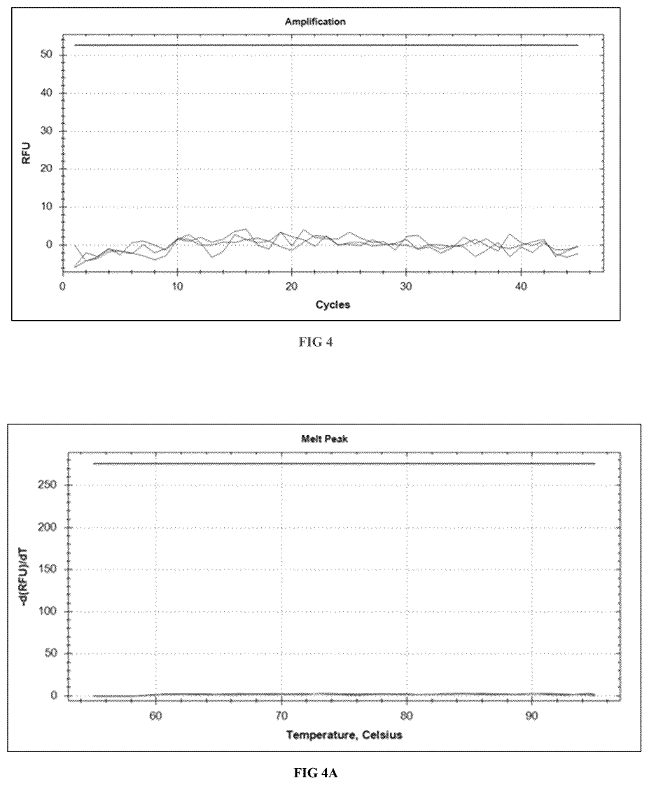

FIG. 4 shows amplification curves for no template control reactions using primer sequences SEQ ID NO.: 8 and 10;

FIG. 4A shows respective melt curves;

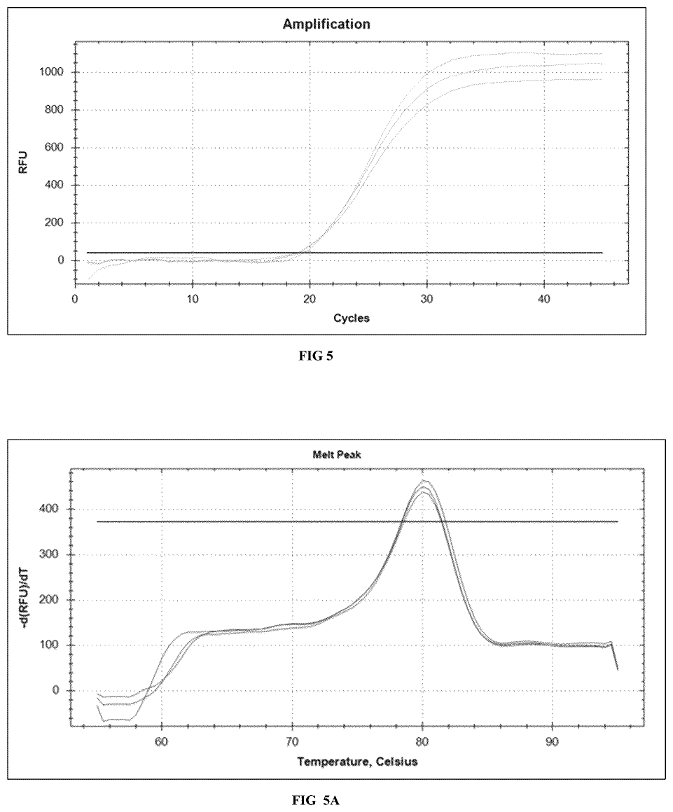

FIG. 5 shows amplification curves for the samples (1 ng DNA) and primer sequences SEQ ID NO.: 9 and 10;

FIG. 5A shows respective melt curves;

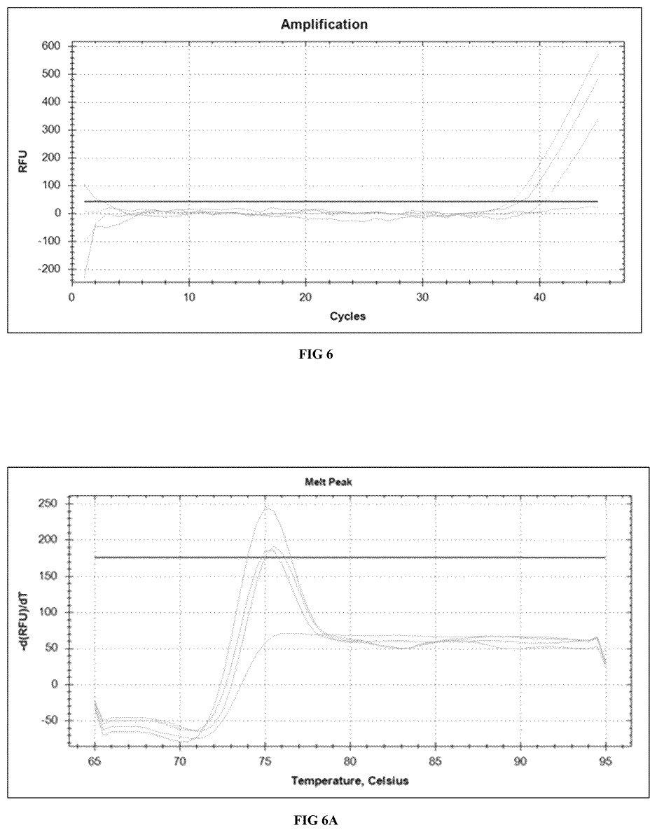

FIG. 6 shows amplification curves for no template control reactions using primer sequences SEQ ID NO.: 9and 10;

FIG. 6A shows respective melt curves;

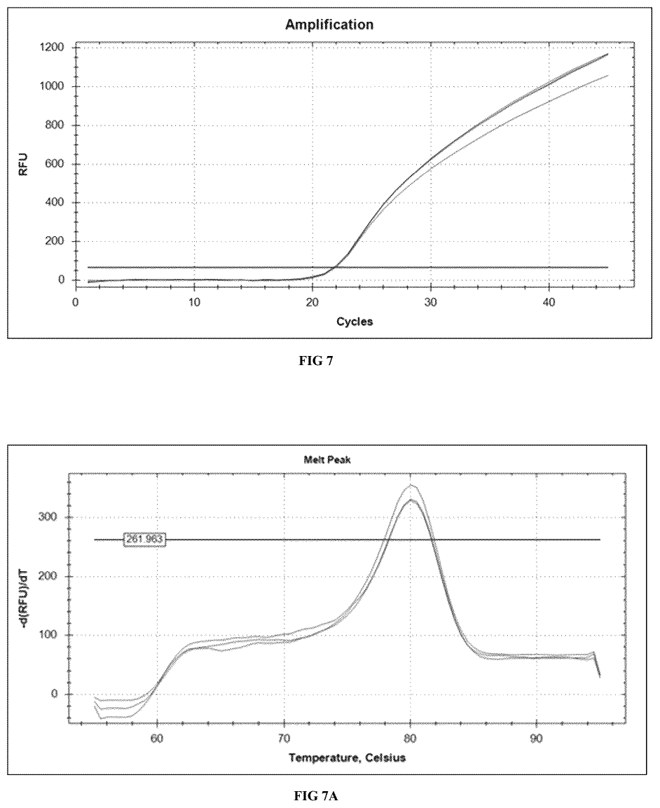

FIG. 7 amplification curves for the samples (1 ng DNA) and primer sequences SEQ ID NO.: 7 and 10;

FIG. 7A shows respective melt curves;

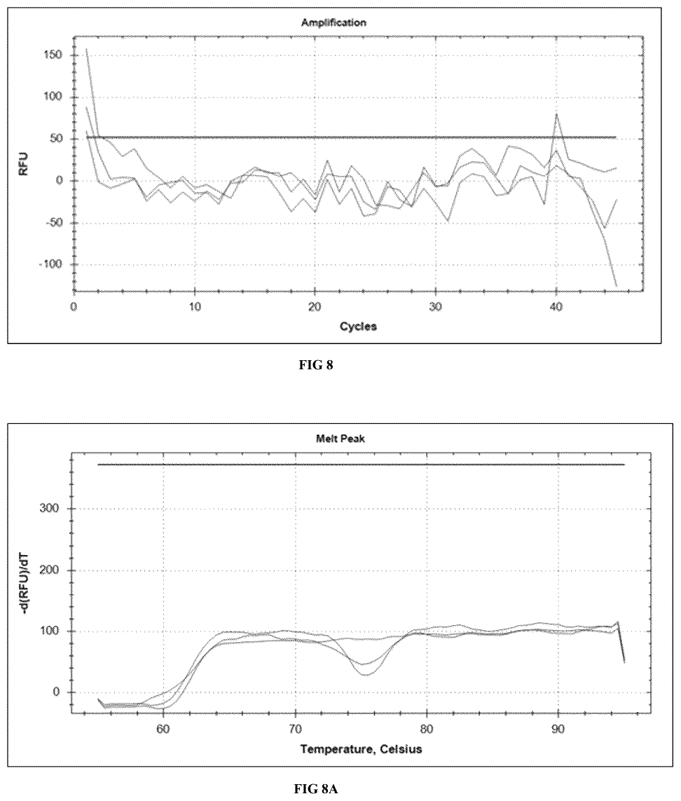

FIG. 8 shows amplification curves for no template control reactions using primer sequences SEQ ID NO.: 7 and 10;

FIG. 8A shows respective melt curves;

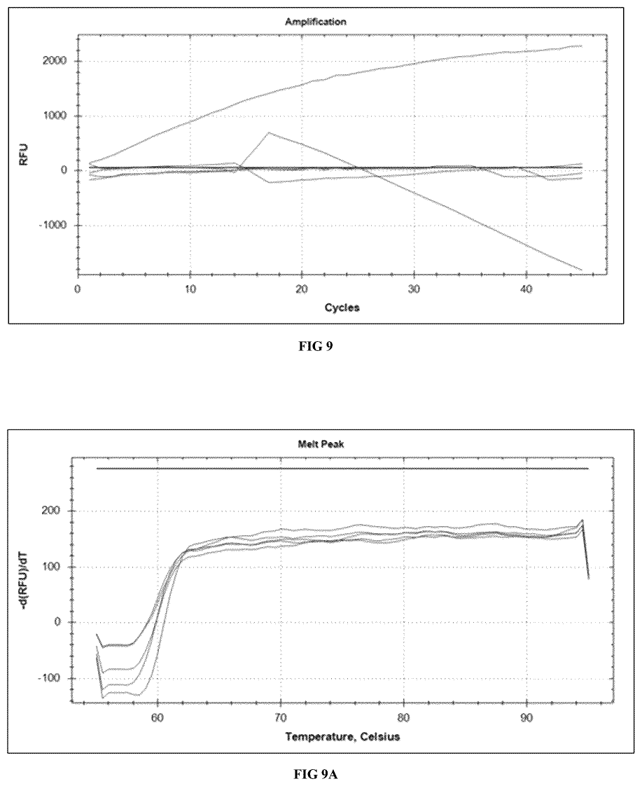

FIG. 9 shows amplification curves for amplification without template using 0.4 uM concentration of FAM labelled primer SEQ ID NO.: 9;

FIG. 9A shows respective melt curves;

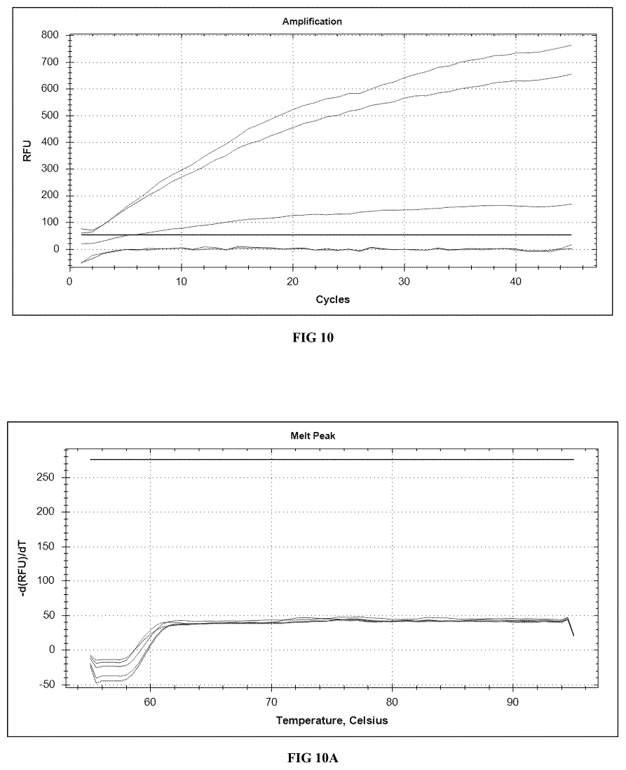

FIG. 10 shows amplification curves for amplification without template using 0.1 uM concentration of FAM labelled primer SEQ ID NO.: 9 and 0.3 uM conc of SEQ ID NO.: 10;

FIG. 10A shows melt curve for the same;

FIG. 11 shows amplification curves for amplification without template using 0.2 uM concentration of FAM labelled primer SEQ ID NO.: 9 and 0.3 uM conc of SEQ ID NO.: 10;

FIG. 11A shows melt curve for the same;

FIG. 12 shows amplification curves for amplification without template using 0.2 uM concentration of FAM labelled primer SEQ ID NO.: 9 and 0.2 uM conc of SEQ ID NO.: 10;

FIG. 12A shows melt curve for the same;

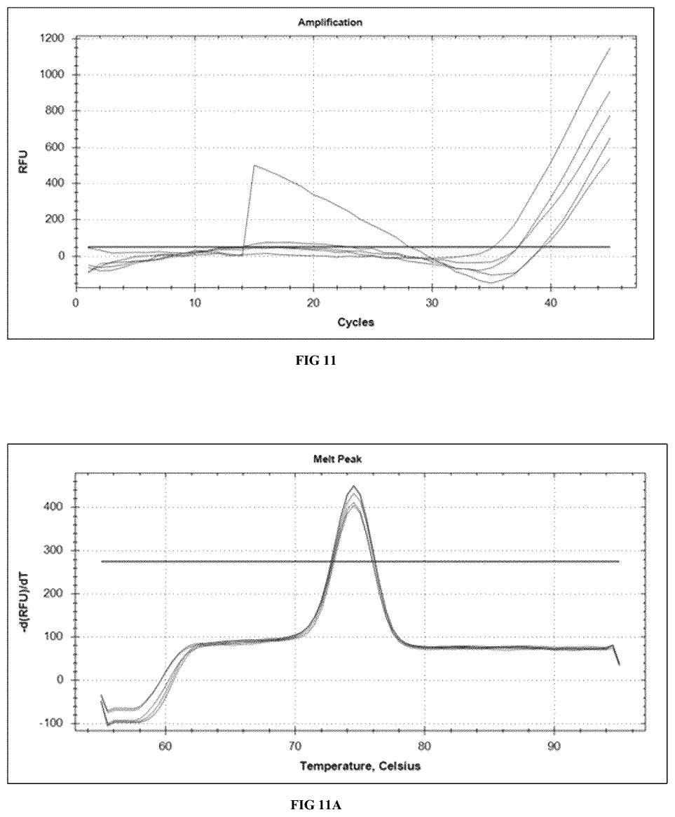

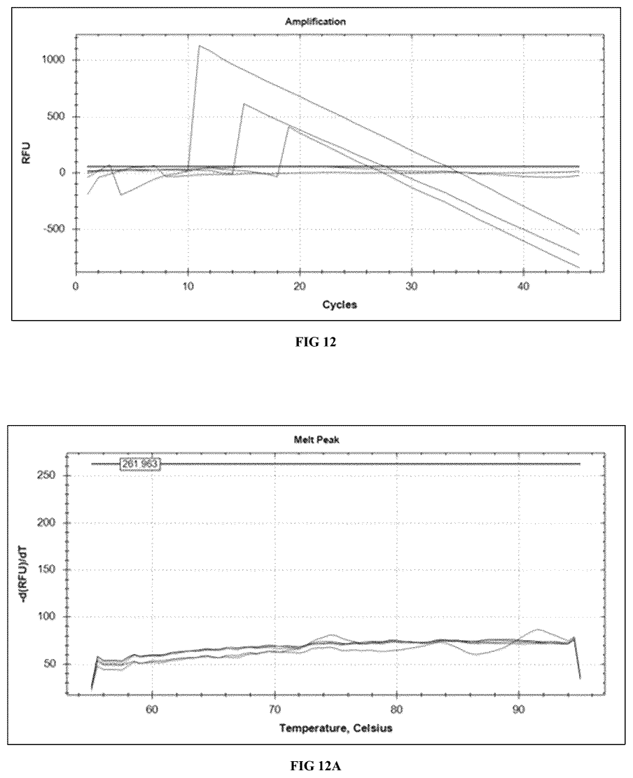

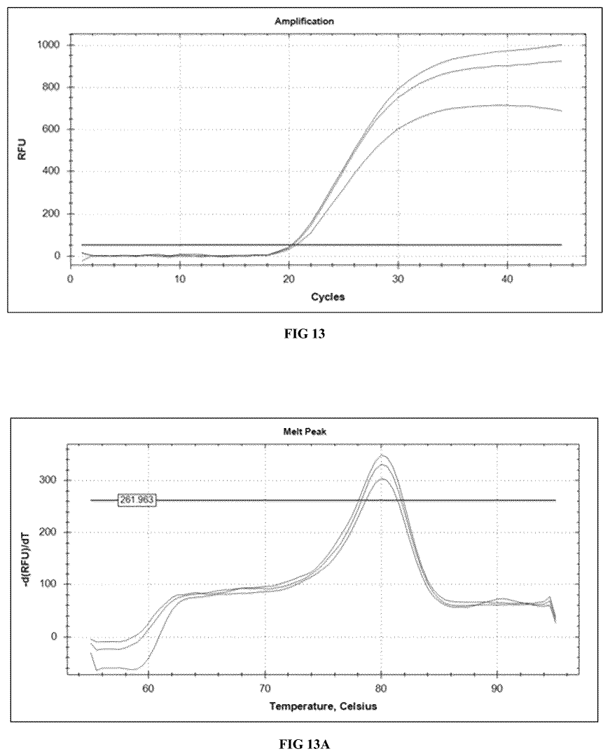

FIG. 13 shows amplification curves for amplification with Ing template DNA using 0.1 uM concentration of FAM labelled primer SEQ ID NO.: 9 and 0.3 uM conc of SEQ ID NO.: 10;

FIG. 13A shows melt curve for the same;

FIG. 14 amplification curves for amplification with 1 ng template DNA using 0.2 uM concentration of FAM labelled primer SEQ ID NO.: 9 and 0.3 uM conc of SEQ ID NO.: 10;

FIG. 14A shows melt curve for the same;

FIG. 15 shows amplification curves for Fluorescein labelled probe and Blackhole Quencher 1 labelled primer-based amplifications of E coli housekeeping gene threonine synthase;

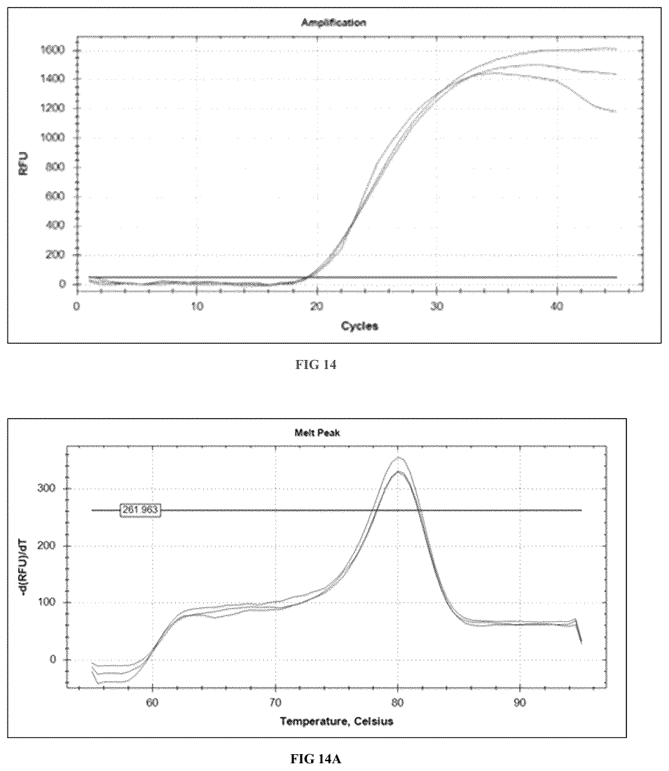

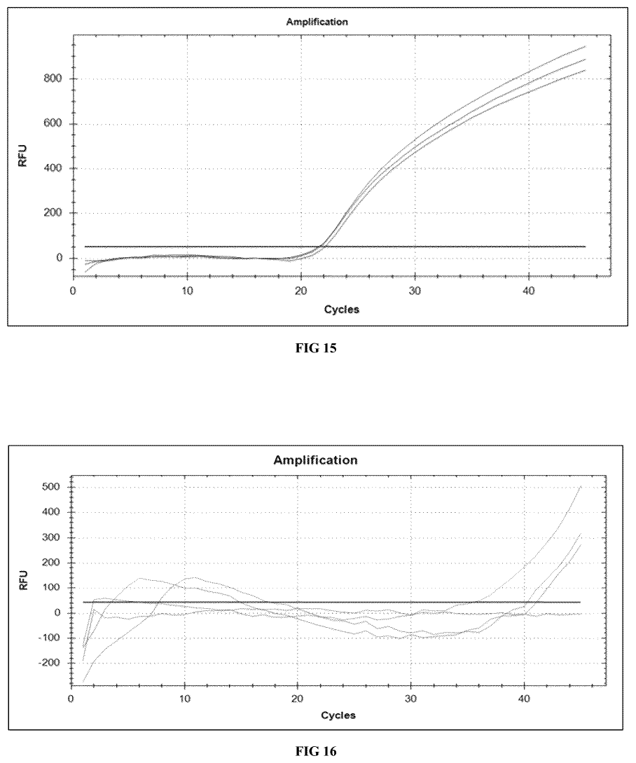

FIG. 16 shows amplification curves for Fluorescein labelled probe and Blackhole Quencher 1 labelled primer-based amplifications of E coli housekeeping gene threonine synthase;

FIG. 16A shows no template control reaction melt curve;

FIG. 17 shows amplification curves for Fluorescein labelled probe and Blackhole Quencher 1 labelled primer-based amplifications of E coli housekeeping gene threonine synthase;

FIG. 18 shows amplification curves for Fluorescein labelled probe and Blackhole Quencher 1 labelled primer-based amplifications of E coli housekeeping gene threonine synthase;

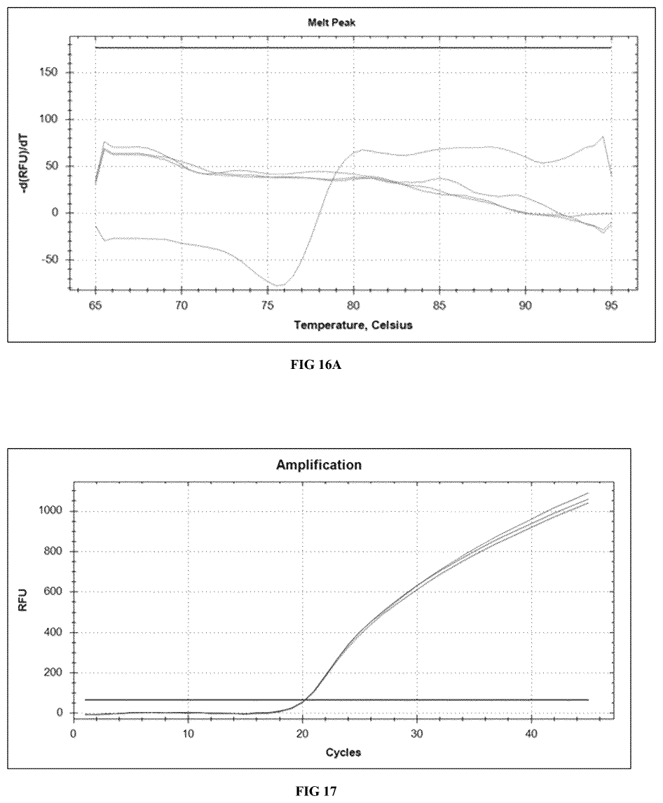

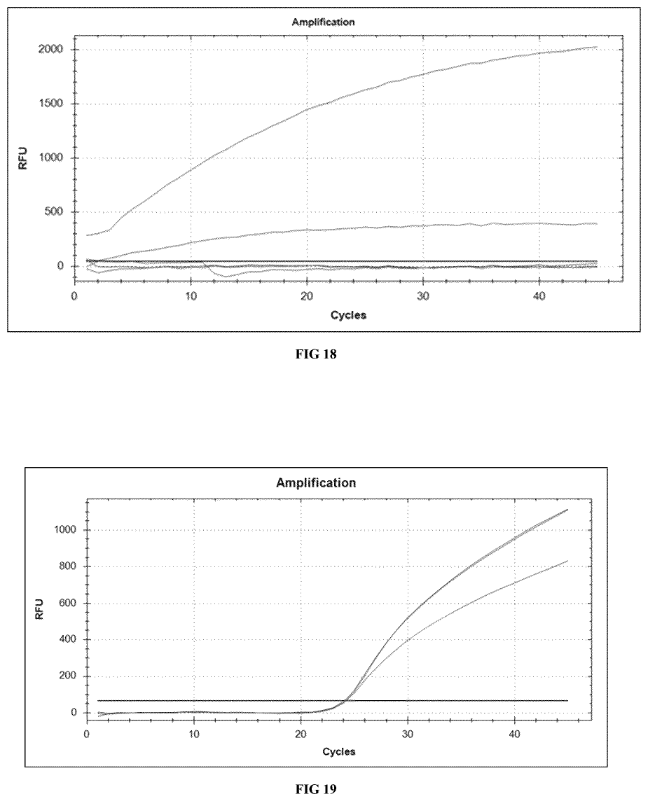

FIG. 19 shows amplification curves for Fluorescein labelled probe and Blackhole Quencher 1 labelled primer-based amplifications of E coli housekeeping gene threonine synthase;

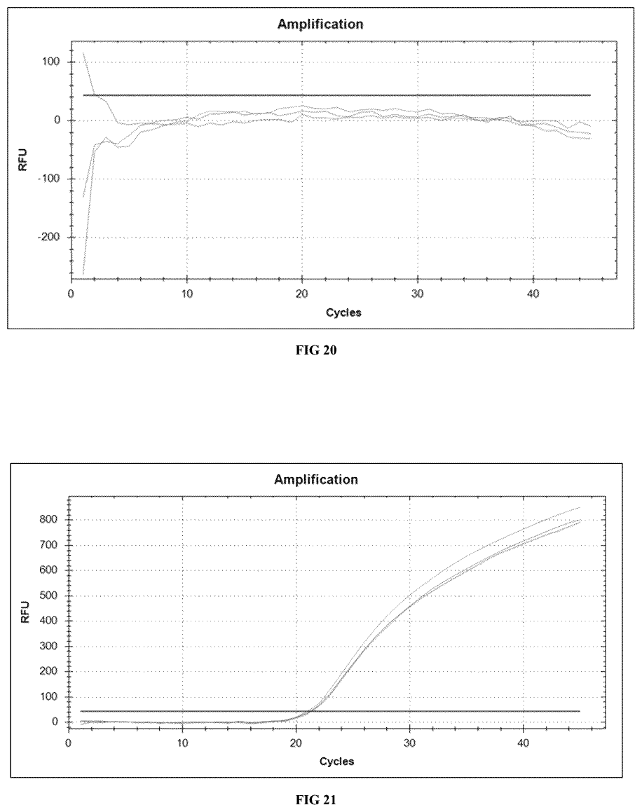

FIG. 20 shows an amplification curve for Fuorescein and BHQ1 dual labeled Taqman probe based assay of E coli housekeeping gene honoserine kinase;

FIG. 21 shows an amplification curve for Fluorescein and BHQ1 dual labeled Taqman probe based assay of E coli housekeeping gene homoserine kinase;

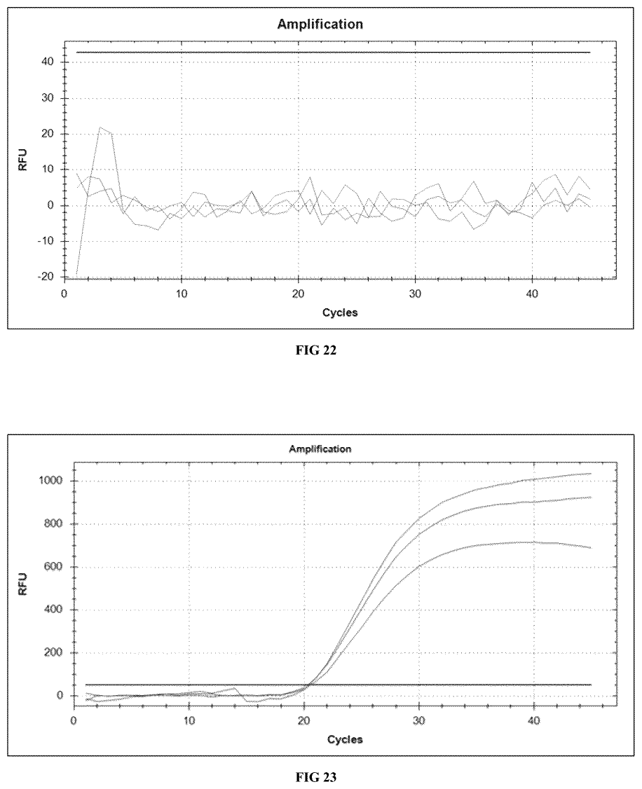

FIG. 22 shows rno ternmplate control reaction for Taqman assay;

FIG. 23 shows amplification curves for the sample and control reaction for amplification of E coli threonine synthase gene target using common non-target primer pair;

FIG. 24 shows amplification curves for the sample and control reaction for amplification of E coli threonine synthase gene target using common non-target primer pair;

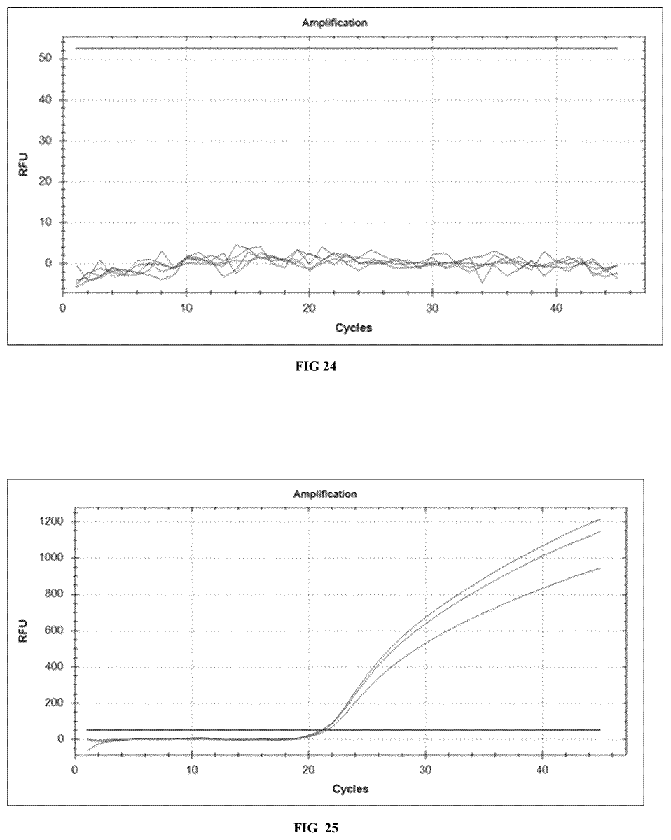

FIG. 25 shows amplification curves for the sample and control reaction for amplification of E coli threonine synthase gene target to generate a FRET signal (Donor fluorophore is excited and emission of acceptor fluorophore is measured) with a well separated melting curve for higher specificity; and

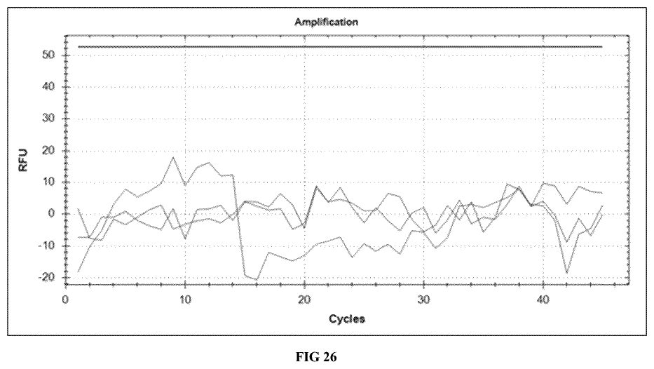

FIG. 26 shows amplification curves for the sample and control reaction for amplification of E coli threonine synthase gene target to generate a FRET signal (Donor fluorophore is excited and emission of acceptor fluorophore is measured) with a well separated melting curve for higher specificity.

### DETAILED DESCRIPTION

Thus, according to the basic aspect in embodiments of the present invention there is provided a method for nucleic acid amplification for target detection with higher sensitivity and higher specificity in comparison to other existing methods. Embodiments of the invention do not try to reduce the formation of primer-dimer or primer dimer like non-specific amplification products, rather it tries to reduce or eliminate signal from primer-dimer and primer dimer like non-specific amplification product. As a result, amplification reaction become free of any non-specific signal and hence results in higher specificity and sensitivity (specificity and sensitivity are interdependent, attempt to increase specificity results in decrease of sensitivity and vice versa). Use of currently available hot start procedure for reduction of non-specific amplification product formation though not required but can be used with the present solution as an addition. Embodiments of the invention disclose a detection method to give specificity 95% or more and sensitivity more than that of the Taqman chemistry (70-80%), the gold standard of the field, i.e., more than 80%, for example, more than 90%. It increases sensitivity by increasing specificity as well as increasing signal for the target amplification product.

Primers and probes of existing/current methods and other methods in the art generate signal from target amplification product as well as from non-specific primer dimer and primer dimer like amplification products. Any increase in signal will increase signal for both target amplification and non-specific amplification products not making any impact on signal to noise ratio. Whereas primers and probe of the present method is specially designed to generate signal from the target amplification product only and to avoid signal generation from the non-specific primer dimer and primer dimer like amplification products.

Moreover, primers and probe of the present method is additionally designed to result in higher signal from target amplification product depending on position and neighboring base sequence of the donor fluorophore labelled base of labelled primer or probe. Therefore, increase in signal from target amplification product in addition to nil or near signal from primer dimer and primer dimer like non-specific products will result in higher sigher signal to noise ratio and hence higher detection sensitivity and specificity.

Primers and probe of present method are labelled with donor fluorophore and/or acceptor fluorophore (radiative quencher)/quencher and are so selected, designed and labelled that the donor fluorophore and the acceptor fluorophore (radiative quencher)/quencher remain well separated and beyond their FRET (energy transfer) distance in target amplification product so that there is no energy transfer interaction between them and a fluorescent signal is generated on target amplification due to enhancement of donor fluorophore fluorescence.

Whereas in primer dimer or primer dimer like non-specific amplification products, donor fluorophore and acceptor fluorophore (radiative quencher)/quencher come within their FRET (energy transfer) distance resulting in energy transfer from donor fluorophore to acceptor fluorophore (radiative quencher)/quencher and quenching of donor fluorophore fluorescence. There is simultaneous enhancement and quenching of donor fluorophore fluorescence in such products. The extent of quenching of donor fluorophore fluorescence depends on spectral properties of donor fluorophore and acceptor fluorophore (radiative quencher)/quencher and their separation in primer dimer or primer dimer like product, and lengths of linkers used for attaching the donor fluorophore and the acceptor fluorophore (radiative quencher)/quencher.

When the extent of quenching is more than the enhancement, no signal will be generated from these non-specific products. As a result, specificity will be high but there will be loss of sensitivity as the excess quenching in primer dimer or primer dimer like product will reduce overall fluorescent signal and hence signal generated by the target amplification product will get reduced. In this case specificity will be the highest but sensitivity will be slightly reduced

When enhancement and quenching of donor fluorophore fluorescence in primer dimer or primer dimer like non-specific products are equal or balanced, no signal is generated by these non-specific products and no fluorescent signal of target amplification product is lost; as a result, both specificity and sensitivity will be the highest.

On the other hand, when quenching is less than the enhancement, there will be a signal from these non-specific products resulting in loss of specificity but there will be no loss of sensitivity. In this case specificity will be slightly reduced but sensitivity will be the highest. This choice should be last among the three choices available in the present method but can be used in certain situations. Because of this balancing of enhancement and quenching of donor fluorophore fluorescence, the labelled primers and probe of embodiments of the invention are named Libra primer pair and Libra primer—probe pair. This mechanism will not work in existing primers and probes; it is specific to the present design and method.

Primers and probe are labelled with donor fluorophore and acceptor fluorophore and are so selected, designed and labelled that the donor fluorophore and the acceptor fluorophore are placed within their FRET distance in target amplification product resulting in energy transfer from the donor fluorophore to the acceptor fluorophore and a FRET signal from the acceptor fluorophore (exciting donor fluorophore with radiation of wave length specific for excitation of donor fluorophore and measuring fluorescence of acceptor fluorophore) is generated on target amplification and, in primer dimer or primer dimer like non-specific amplification products, the donor fluorophore and the acceptor fluorophore come within their short range energy transfer distancei.e., static/contact quenching distance (donor fluorophore and acceptor fluorophore labelled bases are placed opposite to each other or at a separation of 0-3 bases, for example, 0-1 bases between them resulting in energy transfer from the donor fluorophore to the acceptor fluorophore and quenching of donor fluorophore fluorescence as well as acceptor fluorophore fluorescence resulting in a nil or near nil signal from the primer dimer or primer dimer like products.

The target nucleic acid sequence is a nucleic acid sequence (natural or artificial), single or double stranded, nucleic acid amplification product, methylated DNA, mitochondrial DNA, C—DNA (complementary DNA), exosome DNA or the sequence of an infectious agent, genomic DNA (g DNA), mutation (single base change or deletion or insertion of a few bases or long sequences) of genomic sequence or genomic sequence of bacteria, yeast, human, animal, plant and their pathogens including viruses or any other organism, live or dead, RNA, messenger RNA (m-RNA), ribosomal RNA (r-RNA), small RNA, transfer RNA (t-RNA), micro-RNA (mi-RNA), micro-RNA precursor (pre-/pri-mi-RNA), the presence or absence of which are implicated to the presence of infectious agent, disorder or disease or susceptibility to infection or disease or disorder or suitability to disease treatment, genetic trait, prenatal diagnosis, genotype, cell type, tissue type, allele type, SNP detection species or strain type, cancer type, cancer sub-type, cancer detection, disease type or sub-type, expressed gene etc, but not limited to the above.

Another objective is to detect polynucleotides (nucleic acid) or non-nucleic acid analytes present in biological or non-biological samples including but not limited to the clinical samples including but not limited to the body fluids like blood, urine, lymph fluid, saliva, cerebrospinal (CSF) fluid, bronchial wash, perspiration, ascitic fluid, amniotic fluid, sputum, faeces, pus, semen, vaginal swabs, throat and nasal swabs, nodules, tissue samples, tumour samples, biopsy samples, liquid biopsy samples, circulating tumour cells, circulating stem cells, very small embryonic like stem cells, stem cells, cancer cells, cancer stem cells, culture media, fermentation broth, soil, water, food, petroleum well samples, forensic samples etc. The polynucleotides/nucleic acids, non-nucleic acid analytes may be not purified or purified or partially purified by any of the known methods of nucleic acid purification or extraction or other methods.

The following definitions are provided for specific terms used in the following description and the appended claims to more clearly and concisely describe and bring out the subject matter of the present disclosure.

The words “a” or “an” in this specification means at least one, unless specifically stated otherwise, singular includes the plural. As an example, but not as a limitation “a target nucleic acid” means that more than one or more copies of a particular species of target nucleic acid species as well as two or more number of different target nucleic acid species. “and/or” means that the terms before and after the slash can be taken together or separately. For illustration purpose but not as a limitation, A and/or B can mean A and B or A or B.

The words “Nil signal” in this specification means no detectable signal and “near nil signal” means insignificant or in-substantial signal.

It will be appreciated that there is an implied “about,” “almost” prior to the concentrations, distance, times, quantity, temperature, etc discussed in the present specification, where slight and insubstantial deviations are construed to be within the scope of the present teachings herein.

Further, the use of the words “contain,” “contains”, “containing”, “include”, “includes” and “including,” “comprise”, “comprises”, “comprising”, “consist” “consists”, “consisting” are not intended to limit the scope of the present teaching. It is to be understood that the foregoing general description and detailed description are exemplary and explanatory only and are not restrictive of the teachings.

The terms “nucleic acid”, “polynucleotide,” “oligonucleotide “are used interchangeably and refer to single-stranded and double stranded polymers of nucleotide monomers, including without limitation, entirely 2′-deoxynucleotides (DNA) or entirely ribonucleotides (RNA) or a chimeric mixtures thereof and may include nucleotide analogue and the nucleotide monomers are linked by internucleotide phophodiester bond linkages, or internucleotide analogues and associated counterions including H+, NH4+, trialkylammonium, Mg2+, Na+ and the like.

The terms “strand,” “nucleic acid strand,” used interchangeably refers to a single polynucleotide chain of deoxynucleotides or ribonucleotides.

Synthetic or semi-synthetic nucleic acids are synthesized in laboratory chemically or biochemically using their nucleotide building blocks (by PCR or biochemical reactions other than PCR or adding synthetic sequences to natural sequence).

Oligonucleotides are a small length of ribose-nucleic or deoxyribose-nucleic acid polymers synthesized chemically in laboratory using their building blocks, or generated biochemically in laboratory from natural nucleic acid molecules. It has two hydroxyl moieties at its two ends namely 5′ and 3′ ends.

Polynucleotides typically range in size of 5-40 nucleotides where they are sometimes referred to in the art as oligonucleotides and can be of the size of several thousand monomeric units. Whenever polynucleotides are represented, it is understood if not stated otherwise that the nucleotides are in the 5′-3′ direction from left to right where “A” denotes deoxy-adenosine, “G” denotes deoxy-guanosine, “C” denotes deoxy cytosine “U” denotes deoxy-uridine and “T” denotes deoxy-thymidine. Further, ribose or deoxyribose nucleic acid and ribonucleotide and 2′—deoxynucleotide mean the same.

The term “nucleotide” refers to a phosphate ester of nucleoside, e.g, triphosphate ester, where most common site of esterification is C5 position of the pentose sugar. Term “nucleosides” refer to a compound containing a purine or deazapurine base like adenine, guanine, deazaadenine, deazaguanine or pyrimidine base like cytosine, uracil, pseudo-uracil, thymine, ionisine or the like and linked to a pentose sugar at its 1′ position, including 2′deoxy and 2′ hydroxyl forms where the pentose bases are attached to 9 position of purine bases and 1 position of pyrimidine bases.

Artificial nucleotides are modified synthetic nucleotides wherein, either the purine-pyrimidine base or the sugar moiety or the phosphate group is modified. Exemplary base analogues include pseudouridine, 2,6 diaminopurine, hypoxanthine, isoguanine, isocytosine, 2-thiopyrimidine, C-5 propyene, exemplary sugar analogues include 2′ or 3′ position of sugar modified with hydrogen, hydroxy, alkoxy, e.g., methoxy, ethoxy, allyloxy, butoxy, isobutoxy, isopropoxy and phenoxy, azido, amino or alkylamino, fluoro, chloro and bromo and include locked nucleotide (2′ and 4′ hydroxyl groups of the ribose sugar moiety of a ribonucleotide are joined together thus locking them—LNA). Phosphate analogues have one or more of the oxygen atoms replaced with non-oxygen moiety, e.g., sulphur, selenium, boron. Exemplary phosphate analogues include phosphorothioate, phosphorodithioate, phosphorselenoate, phosphorodiselonoate.

Phosphoranilinothioate, phosphoranilidate, phosphoramidite, boronophosphates, including associated counterions and peptide nucleotides (a neutral peptide like N-(2-aminoethyl) glycine backbone is attached on the phosphate group—PNA).

Hapten: Haptens are small molecules that do not elicit an immune response of its own but elicit immune response when attached to a carrier molecule like a protein or a peptide, the carrier molecule as such may or may not be immunogenic itself.

Aptamers: Aptamers are single stranded DNA or RNA or synthetic nucleic acid with modified base or sugar moiety/moieties of short length.

The terms “amplified product”, “amplification product” or “amplicon” refer to a fragment of DNA amplified by a polymerase using a pair of primers in an amplification method such as PCR.

The terms “target amplification product,” “specific amplification product,” “target specific amplification product,” “specific amplification product,” “specific target amplification product,” are used interchangeably and refers to a fragment of a particular nucleic acid amplified by a polymerase using a pair of primers in an amplification method such as PCR.

The terms “target,” “target sequence,” “target nucleic acid,” “specific target,” “target specific,” “target nucleic acid sequence,” “specific sequence,” “target specific sequence,” “specific nucleic acid,” “specific nucleic acid sequence,” “nucleic acid of interest,” “template,” are used interchangeably and refers to a desired region or a desired region of a particular nucleic acid to be either amplified, detected or both or amplified and quantified.

The terms “identical,” “identical sequence” is used interchangeably and means two nucleic acid sequences having the same sequence or a complementary sequence.

“Complementary sequence,” is a complement of a nucleic acid sequence where the nucleotide bases of the complementary sequence pair with the respective complementary bases of the nucleotide sequence, for example guanine base is complementary to cytosine base and adenine base is complementary to thymidine or uracil base. Base complementarity can be full or partial as long as the hybrid is stable. Base complementarity is the ability to base pair. Two sequences are complementary means one sequence complements the other sequence.

“Polymerisation,” “nucleic acid synthesis,” used interchangeably refers to the process of extending the sequence of a primer through the use of a nucleic acid template, a polymerase and nucleotides or through sequential addition of nucleotides.

“Primer,” that has a free extendable 3′ end with a 3′ hydroxyl (OH) group refers to an oligonucleotide synthesized chemically or generated from a bigger nucleic acid molecule that can prime or initiate synthesis or polymerisation reaction extending the primer and producing a primer extension product that is complementary to a nucleic acid strand, at a suitable temperature for a sufficient amount of time in presence of deoxyribonucleotides such as G, C, A and T and a polymerase enzyme such as DNA polymerase or reverse transcriptase and a suitable buffer which includes substituents which are cofactors, or which affect pH, ionic strength etc. Primers selected are at least substantially complementary to hybridize to the respective strands of each specific nucleic acid sequence to be amplified. Primers are normally complementary, except when non-complementary nucleotides may be present at predetermined sequence location, such as a primer terminus as described.

In some embodiments, the primer may be single stranded. In some embodiments, a non-complementary nucleotide sequence or fragment may be attached to the 5′ end of the primer where the remainder of the primer sequence is complementary or sufficiently complementary to the target region of the target nucleic acid. In some embodiments, the non-complementary nucleotide sequence is referred to as non-target sequence.

“Probe” is a non-extendable oligonucleotide attached to a fluorescent reporter dye, or to a fluorescent reporter dye and biotin or the like, or a fluorescent reporter dye and a quencher where the oligonucleotide is complementary to a strand of target nucleic acid that is amplified in a nucleic acid amplification reaction.

The terms “annealing” or “anneal” and “hybridizing” or “hybridize” or “hybridization” without limitation, mean nucleotide base pairing of one nucleic acid with another nucleic acid that results in the formation of a duplex, or higher order structure using A: T, A: U, and G: C, Watson-Crick base pairing.

The terms “amplification,” “target amplification,” “nucleic acid amplification,” are used interchangeably and refers to the use of any amplification process or procedures to increase the concentration of a particular nucleic acid sequence within a mixture of nucleic acid sequence.

The terms “thermal cycling,” “thermal cycles,” “thermal cycle,” typically used in polymerase chain reaction (PCR), a nucleic acid amplification process, refers to repeated cycles of temperature changes from a total denaturing temperature to an annealing/hybridizing temperature, to an extension temperature and back to the total denaturing temperature. In some embodiments, annealing/hybridizing temperature and the extension temperature are combined to a single temperature. The terms also refer to repeated cycles of the above cycle of temperature changes. In some embodiments, the terms a “single cycle” or “single round of cycling” that may be used means one round of above-mentioned sequence or sequences of temperature changes. In some embodiments, single round cycling may include denaturing temperature, repeated cycles of a first annealing/hybridizing temperature and a first extension temperature and repeated cycles of a second annealing/hybridizing temperature and a second extension temperature where the first annealing and extension temperatures are different from the second annealing and extension temperatures or the first annealing temperature is different from the second annealing temperature. No of cycles cane be one or any number up to 45. Total denaturing temperature unwinds a double stranded DNA fragment into two single strands, annealing temperature allows the primer to anneal or hybridize to a complementary strand of the separated strands of the DNA fragment and extension temperature allows synthesis of a nascent DNA strand of the amplification product or amplicon.

In Isothermal amplifications processes, only a single temperature is used for the amplification of target sequence where the separation of the two strands of a double stranded DNA is achieved by the use of primers with a loop structure, which allows primer to anneal to the loop (Loop mediated amplification-LAMP) or by the use of DNA double strand unwinding enzymes like recombinase (Recombinase-polymerase amplification—RPA), helicase (Helicase-polymerase amplification—HPA), topoisomerase (Topoisomerase-polymerase amplification—TPA) and the like, and the polymerase to synthesize the nascent strands of the nucleic acid where the polymerase used in LAMP possesses strand displacement enzyme activity also.

Quantitative PCR (qPCR) or quantitative isothermal amplification means amplification for quantification of a nucleic acid in a reaction mixture or a nucleic acid sample and the amplification signals are acquired at the logarithmic phase of the amplification reaction, when the signal crosses a threshold value.

Hot start PCR generally refers to limiting the availability of an essential reaction component e.g., a polymerase at a first temperature (typically a lower temperature) until a second temperature (typically a higher temperature that is the annealing temperature of primer) is reached that allows the essential component to participate in the amplification reaction.

Reverse Transcription Polymerase Chain Reaction (RT-PCR) is the process of synthesizing a DNA strand from an RNA strand using a reverse transcriptase enzyme and then employing PCR to the synthesized DNA strand. In this process either a combination of a reverse transcriptase and a DNA polymerase or an enzyme that has both reverse transcriptase enzyme activity and polymerase activity are used.

In Allelic Polymerase Chain Reaction (Allelic-PCR), one of the two primers of the amplification reaction carries base mismatch or base mismatches with the target sequence at one or two bases, one to four bases away from its 3′ end where the alleles of a target sequence differ in the sequence of one or two bases, for example, one base, the penultimate base at 3′ end of one of the primers. Additionally, one of the primers is the allelic primer labelled with the acceptor or quencher moiety and a probe labelled with donor fluorophore hybridizes to one of the two strands of the amplified target.

In methylation specific nucleic acid amplification including methylation status PCR or methylation specific PCR, a methylated DNA is treated with the bisulphite reagent, which converts a methyl-cytosine base of the methylated DNA to an uracil base resulting in a change of the sequence of the DNA, which is then amplified with the primers specific for the changed sequence.

In emulsion PCR, PCR amplification of a target sequence is carried in oil—water emulsion where aqueous PCR reaction mixture is diluted with the oil to an extent that each of the water droplets formed contain a few, for example, a single, nucleic acid molecule.

In bead PCR, beads or a bead carries a primer that binds and captures the target nucleic acid and amplifies the target nucleic acid in association with another target specific primer. Either the bead is used in emulsion PCR or each bead with the captured target nucleic acid is captured in a small hole and subjected to PCR amplification.

In droplet or digital PCR (dPCR), a PCR reaction mixture complete with all required ingredients, reaction buffer, nucleotides, enzyme or enzymes, primers or primers and probe and target nucleic acid is partitioned into thousands of individual reaction droplets and amplification signal is acquired at the end point of the amplification reaction. dPCR allows detection of very low copy number of target and absolute quantification of target.

The terms “non-specific signal,” “non-specific fluorescence,” “background fluorescence” used interchangeably refers to the detectable signal emitted from fluorophores or nucleic acid binding dye molecules associated with double stranded nucleic acid other than desired amplification products or amplicons (Non-specific amplification products or non-specific amplicons) and desired amplification products or amplicons comprise the amplification products of target nucleic acids, including in some embodiments, internal standards or positive control sequences that may be included in the reactions of some embodiments.

Nonspecific amplification is the amplification event where an amplification product or products other than the intended amplification products (the specific target amplification products) are generated or produced, and the unintended amplification products are called the non-specific amplification products.

Primer dimer: It is a non-specific amplification product generated or produced when two primer molecules overlap with each other at their extendable 3′ ends and the polymerase of the amplification reaction extend the 3′ ends of two primers molecules using one as template for the other.

Primer dimer like amplification product is a non-specific amplification product generated or produced when the 3′ end sequence of one or both amplification primers overlap with the non-extendable 3′ end sequences of a probe used for monitoring of the amplification reaction and the polymerase of the amplification reaction extends the 3′ ends of the primer(s) molecules using the probe as template. The primer dimer like product so formed is further extended in the amplification process against the strand of the target complementary to the strand to which the probe hybridizes generating an amplifiable primer dimer like product.

The term “denaturing” and “denaturation” as used herein refers to a process in which a double stranded nucleic acid/polynucleotide including without limitation, a genomic DNA (gDNA) fragment, at least one target nucleic acid, a double stranded amplification product or amplicon, a double stranded polynucleotide fragment is converted to two single stranded polynucleotides or to a single stranded or substantially single stranded polynucleotide, as appropriate. Melting temperature, or “Tm,” is a measure of stability of a nucleic acid duplex and is the temperature at which half of the base pairs of a particular nucleic acid duplex have disassociated. “Melting curve” or “melt curve” is a curve or profile of the disassociation and association of the base pairs of a particular nucleic acid duplex, which is normally a plot of-dF/dT vs T where “F” is the measure of fluorescence of the nucleic acid duplex and “T” is the temperature.

The term “label” as used herein refers to any atom or molecule that can be used to provide or aid to provide a detectable or quantifiable signal and can be attached to a nucleic acid or an oligonucleotide, where the detectable signal is a fluorescence signal and labels generating detectable fluorescence are herein referred to as “fluorophore” or “reporter dyes” or “fluorescent dyes” or “dyes,” and labels that absorb or reduce the fluorescence signal of a fluorophore or quench the fluorescence of a fluorophore or a fluorescent dye are quenchers. Quencher can be a fluorophore (acceptor fluorophore or radiative quencher) or a non-radiative quencher or dark quencher (absorbs fluorescence signal but does not emit any light or radiation).

Fluorophores release energy or electromagnetic radiation including light when irradiated with an electromagnetic radiation including light and this release of energy or electromagnetic radiation or light is the emission of the fluorophore. The energy or electromagnetic radiation or light emitted by the fluorophore is different from the energy or electromagnetic radiation it is irradiated with, and is the fluorescence of the fluorophore. Fluorophores absorb light or radiation of a particular wave length or wave lengths and emits its characteristic light or radiation of a different wave length or wave lengths. Fluorescence maximum or emission maximum is the wave length at which fluorescent emission of the fluorophore is maximum. The fluorophore that transfers its energy or light or radiation to an acceptor fluorophore or a quencher is a donor fluorophore. Acceptor fluorophore absorbs the energy of donor fluorophore releases its characteristic energy or electromagnetic radiation or light different from that of the donor and is also called a radiative quencher. Whereas dark quencher or non-radiative quencher absorbs energy or electromagnetic radiation or light of donor and do not release any electromagnetic radiation or light.

The terms “Reporter dyes,” “fluorescent dyes”, “dyes,” which also include “nucleic acid binding dyes,” or “intercalating dyes” and these terms are used interchangeably. Nucleic acid binding dyes refers to a fluorescent molecule specific for a double stranded polynucleotide or that at least shows a substantially greater enhancement of fluorescence when associated with a double-stranded polynucleotide than with a single stranded polynucleotide. Non-limiting examples of nucleic acid binding dyes include ethidium bromide, Hoechst dye 33342, Hoechst dye 33258, DAPI, Lanthanide chelates (NDI-(BH-HCT-Eu3+chelates) and unsymmetrical cyanine dyes SYBR(R)Green and Pico(R)Green, Avagreen®, YOPRO 1, SYTO 9, Acridine Orange.

Excitation spectrum of a fluorophore is a graphical presentation of the energy of the electromagnetic radiation absorbed by the fluorophore across the spectral wave length range of electromagnetic radiation, and emission spectrum of a fluorophore is the energy of the electromagnetic radiation emitted by the fluorophore across the spectral wave length range of electromagnetic radiation.

Spectral overlap is the overlap of the excitation spectrum of a fluorophore or a quencher with the emission spectrum of another fluorophore.

Polymerase herein is the enzyme that catalyses the polymerization reaction of nucleotides.

Polymerase can be DNA polymerase or RNA polymerase depending on whether deoxyribonucleotide or ribonucleotide is polymerised. DNA polymerase can be a DNA dependent DNA polymerase or a RNA dependent DNA polymerase. When an RNA polymerase, when it synthesizes a RNA molecule by polymerizing ribonucleotides using single stranded DNA as a template (the process is called RNA transcription), or a reverse transcriptase when it synthesizes a DNA strand by polymerizing deoxy nucleotides using an RNA strand as a template (the process is called reverse transcription). Some reverse transcriptase has both reverse transcriptase enzyme activity as well as DNA polymerase enzyme activity (Thermus thermophilus or Tth polymerase). Some DNA polymerases may have strand displacement activity, exonuclease enzyme activity, non-template dependent polymerase activity. When a polymerase shows its enzyme activity above 370° C. and above up to a temperature 60° C. is a meso-thermostable polymerase and is a thermostable polymerase when the polymerase is active at a temperature above 600° C. When a polymerase shows its enzyme activity at temperature at or below 370° C. is an ambient temperature polymerase (37° C. to 25° C.) or at a temperature below 250C, a low temperature polymerase. A polymerase can be a hot start polymerase, a polymerase that exhibits polymerisation activity at annealing temperature of a primer. Nucleic polymerase may be selected from the group consisting of, but not limited to, Taq DNA polymerase, Pfu DNA polymerase, Vent™ DNA polymerase, Tfl DNA polymerase, Tfi DNA polymerase, Tth DNA polymerase, Tli DNA polymerase, thermostable polymerase with the helicase or DNA unwinding activity

Further, Kits are also provided herein for practising or performing the methods of the present teaching. The term “kit” herein refers to a packaged set of related components or compositions, typically one or more in one or more containers or vessels. The kit may typically comprise at least a pair of oligonucleotides as a pair of primers for polymerizing and/or amplifying at least one target nucleic acid from a sample, where one member of the pair of oligonucleotides is labelled with a detectable label like a fluorophore or an intercalating dye and other member of the pair is labelled with a quencher. The kit may also typically comprise at least a pair of oligonucleotides as a pair of primers for polymerizing and/or amplifying at least one target nucleic acid from a sample, where at least one member of the pair of oligonucleotide primers is labelled with a quencher, and another non-extendable oligonucleotide with a detectable label, a fluorophore or an intercalating dye as a probe for monitoring the amplification reaction.

The kit may also typically comprise at least a pair of oligonucleotides as a pair of primers for polymerizing and/or amplifying at least one target nucleic acid from a sample, where the oligonucleotides are labelled with detectable labels like a fluorophore or an intercalating dye where one member of the fluorophores is a donor fluorophore and other member is an acceptor fluorophore. The kit may also typically comprise at least a pair of oligonucleotides as a pair of primers for polymerizing and/or amplifying at least one target nucleic acid from a sample, where at least one member of the pair of oligonucleotide primers is labelled with a detectable label, and another non-extendable oligonucleotide labelled with another detectable label as a probe for monitoring the amplification reaction, where the detectable labels are a fluorophore or an intercalating dye and the two labels are a donor and acceptor pair. The kit may also contain samples containing pre-defined nucleic acids to be used in control reactions. The kit may also optionally include a reaction mixture or a PCR master mixture containing all the components other than the primers and probes necessary for amplifying at least one target nucleic acid from nucleic acid templates or may also contain stock solutions of buffers, salts, divalent metal ion (Mg2+, Mn,2+dNTPs (dA, dC, dG, dT, dU), enzymes, glycerol, BSA (bovine serum albumin), gelatin, one or more detergents, PVP (polyvinyl pyrrolidone), PEG (polyethylene glycol), necessary for amplifying at least one target nucleic acid from nucleic acid templates, working concentration range of each component is well known in the art and can be further optimized by a person of ordinary skill in the art. In some embodiments, the kit may include multiple primer sets or primer probe sets. In other embodiments of particular systems or kits which would be understood by one skilled in the art are also included or contemplated. The kit may also contain reaction vessel or vessels for carrying out the amplification reaction where the reaction vessel comprises without limitation a microtube or a microcentrifuge tube (0.2/0.5 ml) and the like commonly used in molecular biology laboratories, a well of a multi-well plate, a spot of a glass slide or a silicon wafer, a channel or chamber of a microfluidics device.

The section headings used herein are for organisational purposes only and are not to construed as limiting the desired subject matter in any way. All patents and other literature references incorporated in this specification are expressly incorporated by reference in their entirety for any purpose. In case any of the incorporated literature contradicts any term defined in this specification, this specification controls.

While the present teachings are described in conjunction with various embodiments, it is not intended that the present teaching be limited to such embodiments. On the contrary present teaching encompasses various alternatives, modifications, and equivalents as would be appreciated by others skilled in the art. Throughout the specification, exemplification of specific terms should be considered as non-limiting examples.

Embodiments of the present invention thus provide the process of nucleic acid amplification for enhancement of donor fluorophore fluorescence to generate a signal for a target amplification and a balanced fluorescence enhancement compensating quenching of donor fluorophore fluorescence or a balanced quenching compensating enhancement of donor fluorophore to generate a nil or near nil signal from primer dimer or primer dimer like non-specific nucleic acid amplification products.

Another objective is to balance the enhancement of the donor fluorophore fluorescence and the quenching of the donor fluorophore fluorescence that occur simultaneously in the non-specific primer dimer (formed due to the overlap and extension of the 3′ ends of a donor fluorophore labelled primer and an acceptor fluorophore/quencher labelled primer by the polymerase used in the amplification reaction) and primer dimer like amplification products (formed due to the overlap of the 3′ ends of a donor fluorophore labelled probe and an acceptor fluorophore/quencher labelled primer and extension of the labelled primer by the polymerase using the probe as template) so that no signal is generated by these non-specific products.

Yet another aspect of embodiments of the present invention is to use a donor fluorophore moiety labelled linear primer and an acceptor fluorophore/quencher moiety labelled linear primer for target nucleic acid amplification. Primers are so selected and so labelled that the donor fluorophore moiety and the acceptor fluorophore/quencher moiety are separated enough from each other in the target amplification product, there is no significant energy transfer between the two moieties and there is a significant enhancement of the donor fluorophore fluorescence due to the incorporation of the donor fluorophore labelled primer into the target amplification product and a melting curve and a melting temperature (Tm) is also generated by the target amplification product.

But in the non-specific primer dimer amplification product, the donor fluorophore and the acceptor fluorophore/quencher come within their FRET distance, the acceptor fluorophore/quencher moiety quenches the fluorescence of the donor fluorophore moiety (FIG. 1) and the positions of the donor fluorophore and the acceptor fluorophore/quencher on two primers are so selected that the amount of the quenching of the donor fluorophore fluorescence by the acceptor fluorophore/quencher is equal or almost equal to the amount of the enhancement of the donor fluorophore fluorescence due to the incorporation of the donor fluorophore labelled primer into the primer dimer product. As a result, no signal is generated on formation of the non-specific primer dimer product, the sensitivity of the target detection is not affected due to the quenching of the fluorescence of the donor fluorophore in the primer dimer and a melting curve is also generated by the primer dimer.

Another objective is to suitably select the placing of the donor fluorophore and the acceptor fluorophore/quencher moiety on the two primers on any base, at least two bases away and for example, a greater number of bases away from the 3′ ends except the 5′ end base of the donor fluorophore labelled primer. The donor fluorophore and acceptor fluorophore labelled primer pair are the libra primer pair.

Another objective is that the donor fluorophore and the acceptor fluorophore/quencher are placed on the primers in such a way that the fluorescence of the donor fluorophore in the non-specific primer dimer amplification product is quenched by the acceptor fluorophore/quencher by 1-50 percent, for example, 17-45 percent and as a further example, 30-40 percent depending on the corresponding extent of enhancement of donor fluorophore fluorescence on probe hybridization, which is respectively 1-100 percent, for example, 20-80 percent and as a further example, 40-70 percent.

Another objective is that the no of bases separating 3′ end of the donor fluorophore labelled primer and the donor fluorophore labelled base, plus the number of bases separating 3′ end of the acceptor fluorophore/quencher labelled primer and the acceptor fluorophore/quencher labelled base, minus the number of bases at the 3′ ends of the labelled primers overlapping with each other in primer dimer is equal to the number of bases equivalent to the distance for the energy transfer of 1-50 percent, for example, 17
45 percent and as a further example, 30-40 percent between the donor fluorophore moiety and the acceptor fluorophore/quencher moiety.

Another objective is that the distance between the donor fluorophore labelled base and the 3′ end of the donor fluorophore labelled primer, plus the distance between the acceptor fluorophore/quencher labelled base and the 3′ end of the acceptor fluorophore/quencher labelled primer, minus the distance of overlap between the bases at the 3′ ends of the primers in primer dimer is equal to the distance of the energy transfer of 1-50 percent, for example, 17-45 percent and as a further example, 30-40 percent between the donor fluorophore moiety and the acceptor fluorophore/quencher moiety.

In another aspect the donor fluorophore labelled primer is labelled with two fluorophores, which can be the same entity or a different entity. This would result in FRET between the two fluorophores and further increase in enhanced signal of amplification product.

Another aspect of embodiments of the present invention is to use a donor fluorophore moiety labelled linear probe and a pair of linear primers for target nucleic acid amplification where an acceptor fluorophore/quencher moiety is placed on one or both primers. Primer and probe are so selected and so labelled that the donor fluorophore moiety and the acceptor fluorophore/quencher moiety are separated enough from each other when the probe hybridizes either to the strand of the target amplification product into which the acceptor/quencher labelled primer got incorporated or to the strand of the target to which the acceptor fluorophore/quencher labelled primer hybridizes/anneals, there is no significant energy transfer between the two moieties and there is a significant enhancement of the donor fluorophore fluorescence on hybridization of the labelled probe. The labelled probe generates a melting curve and melting temperature specific for the melting of the probe from the target amplification product.

But in the non-specific primer dimer like amplification product (formed due to the overlap of the 3′ ends of the labelled probe and the labelled primer(s) and extension of the labelled primer by the polymerase used in the amplification reaction using the probe as template), the acceptor fluorophore/quencher labelled primer gets incorporated into one strand of the primer dimer like product, the donor fluorophore labelled probe hybridizes to the strand of the primer dimer like product into which the acceptor / quencher labelled primer got incorporated (FIG. 2). Hybridization of the donor fluorophore labelled probe results in enhancement of the donor fluorophore fluorescence and brings the donor fluorophore and the acceptor fluorophore/quencher within their FRET/energy transfer distance, the acceptor fluorophore/quencher moiety quenches the fluorescence of the donor fluorophore moiety; positions of the donor fluorophore and the acceptor fluorophore/quencher on probe and primer(s) are so selected that the amount of quenching of the donor fluorophore fluorescence by the acceptor fluorophore/quencher is equal or almost equal to the amount of enhancement of the donor fluorophore fluorescence. As a result, no signal is generated on formation of the non-specific primer dimer like product, the sensitivity of the target detection is not affected due to the quenching of the fluorescence of the donor fluorophore in the primer dimer like product.

Further, the donor fluorophore labelled probe can additionally have a MGB dye (minor groove binder) and the probe is a MGB probe. The term “minor groove binding” as used herein refers to a small molecule that fits into the minor groove of double stranded DNA, sometimes in a sequence specific manner.

Generally minor groove binders are long, flat molecules that can adopt a crescent like shape and thus fit snugly into the minor groove of a double helix, often displacing water. Minor groove binding molecules typically comprise several aromatic rings connected by bonds with torsional freedom, for example, but not limited to, furan, benzene, or pyrrole rings.

Another objective is to suitably select the placing of the donor fluorophore moiety on the probe at the 3′ end or on any base away from the 3′ end except the 5′ end base position and that of the acceptor fluorophore/quencher moiety on the acceptor fluorophore/quencher labelled primer on any base at least two bases away and for example, a greater number of bases away from the 3′ end of the acceptor fluorophore/quencher labelled primer. The donor fluorophore and acceptor fluorophore/quencher labelled probe primer pair are the libra probe-primer pair.

Further objective is to use both primers of the probe-based detection labelled with acceptor fluorophore/quencher moieties where the acceptor fluorophore/quencher moieties on two primers can be the same or different.

In another aspect the donor fluorophore and the acceptor fluorophore/quencher are placed respectively on the probe and the primer in such a way that on hybridization of the donor fluorophore labelled probe to the non-specific primer dimer like amplification product, fluorescence of the donor fluorophore is quenched by the acceptor fluorophore/quencher by 1-50 percent, for example, by 17-45 percent and as a further example, 30-40 percent depending on the corresponding extent of enhancement of donor fluorophore fluorescence on probe hybridization, which is respectively 1-100 percent, for example, 20-80 percent and as a further example, 40-70 percent.

Another objective is that the no of bases separating 3′ end of the donor fluorophore labelled probe and the donor fluorophore labelled base, plus the number of bases separating 3′ end of the acceptor fluorophore/quencher labelled primer and the acceptor fluorophore/quencher labelled base, minus the number bases at the 3′ end of the acceptor fluorophore/quencher labelled primer overlapping with the 3′ end bases of the donor fluorophore labelled probe in primer dimer like product is equal to the number of bases equivalent to the resonance energy transfer/energy transfer distance of 1-50 percent, for example, 17-45 percent and as a further example, 30-40 percent between the donor fluorophore moiety and the acceptor/quencher moiety.

Another objective is that the distance between the donor fluorophore labelled base and the 3′ end of the donor fluorophore labelled probe plus the distance between the acceptor/quencher labelled base and the 3′ end of the acceptor/quencher labelled primer minus the distance of overlap between the 3′ end bases of the labelled primer with the 3′ end bases of the probe in primer dimer like product is equal to the distance of resonance energy transfer of 1-50 percent, for example, 17-45 percent and as a further example, 30-40 percent between the donor fluorophore moiety and the acceptor/quencher moiety.

In a specific extension of the embodiment, the donor fluorophore labelled probe is provided linked to one of the two amplification primers with a non-nucleotide organic linker (non-nucleotide organic linker make the progressing polymerase to fall from the template) like hexamethylene or hexa-polyethylene glycol, for example, hexa-polyethylene glycol or their chimera or their longer length where the probe is designed to hybridize to the nascent target amplification or target amplicon strand synthesized by the probe linked primer.

In another extension, the donor fluorophore labelled probe is labelled with two fluorophores, which can be the same entity or a different entity. This would result in FRET and further increase in enhanced signal of the fluorophore labelled probe.

Fluorescence of the donor fluorophore is usually enhanced by 1-100 percent, for example, 20-80, as a further example 40-70 percent on incorporation of a donor fluorophore labelled primer into an amplification product or hybridization of a donor fluorophore labelled probe to an amplification product or a target strand depending on the nucleotide sequence of the primer or probe around the donor fluorophore labelled base, presence of guanine bases around donor fluorophore labelled base, the position of the donor fluorophore on the donor fluorophore labelled primer or probe with respect to the 3′ end of the donor fluorophore labelled primer or probe, the linkers used for attaching the donor fluorophore and the acceptor fluorophore/quencher to the primer or the probe, secondary structure of the donor fluorophore labelled primer or probe, efficiency of labelling of primers or probe, reaction condition applied.

In non-specific primer dimer or primer dimer like amplification product, the fluorescence of the donor fluorophore of primer or probe is quenched by the acceptor fluorophore/quencher of the primer by 1-50 percent, for example, by 17-45 percent and as a further example 30-40 percent, to avoid any signal generation by the non-specific amplification product. The extent of quenching of donor fluorophore fluorescence by the acceptor fluorophore/quencher not only depends on the spectral properties of the donor fluorophore and the acceptor fluorophore/quencher and their distance or separation but also on the linkers used for attaching the donor fluorophore and the acceptor fluorophore/quencher to the primers and the probe.

Another objective is to place at the 5′ end of the donor fluorophore labelled primer or probe an additional quencher for the donor fluorophore or 4-8 bases (with or without a quencher) non-target sequence complementary to the bases around the donor fluorophore labelled base. In such situation, the fluorescence of the donor fluorophore is increased by about 3-8-fold when the donor fluorophore labelled primer or probe gets incorporated into or hybridized to an amplification product. Positions of the donor fluorophore and the acceptor fluorophore/quencher on the primers or primer and probe are so selected that the donor fluorophore fluorescence is quenched by 65-90 percent in the primer dimer or primer dimer like products. The no of bases separating 3′ end of the donor fluorophore labelled primer or probe and the base carrying the donor fluorophore, plus the number of bases separating 3′ end of the acceptor fluorophore / quencher labelled primer and the base carrying the acceptor fluorophore/quencher, minus the number bases overlapping at the 3′ ends of the labelled primers or labelled primer and probe in primer dimer or primer dimer like products is equal to the number of bases equivalent to the distance for the resonance energy transfer/energy transfer of 65-90 percent between the donor fluorophore moiety and the acceptor fluorophore/quencher moiety.

The distance between the base carrying the donor fluorophore and the 3′ end of the donor fluorophore labelled primer or probe, plus the distance between the acceptor fluorophore/quencher labelled base and the 3′ end of the acceptor fluorophore/quencher labelled primer, minus the distance of overlap between the bases at the 3′ ends of the labelled primers or labelled primer and probe in primer dimer or primer dimer like products is equal to the distance of the resonance energy transfer/energy transfer of 65-90 percent between the donor fluorophore moiety and the acceptor fluorophore/quencher moiety.

Energy transfer is a process of transfer of energy from a first fluorophore moiety when excited with a suitable electromagnetic radiation, to a second fluorophore or a quencher moiety when there is a sizable overlap of the excitation spectrum of the second fluorophore or quencher with the emission spectrum of the first fluorophore, and the two moieties are in proximity. The first fluorophore that transfers energy is called a donor fluorophore or a donor, the second fluorophore is called an acceptor fluorophore or acceptor of donor fluorophore and the quencher is called an acceptor or a quencher of the donor fluorophore. Energy transfer taking place at a short distance from a donor fluorophore to an acceptor fluorophore or a quencher when they are in very close proximity or in contact with each other is a short-range energy transfer process and the resultant quenching of the donor fluorophore is called static or contact quenching. Fluorescent resonance energy transfer (FRET) is a long-range energy transfer that occurs in solution phase without any contact between the donor fluorophore and the acceptor fluorophore or quencher.

Foster radius value will give an idea of the distance between a donor fluorophore and an acceptor fluorophore/quencher for a particular energy transfer efficiency. Any significant energy transfer is up to a distance of 2 R₀. Further, the distance between donor and acceptor ‘R’ for energy transfer efficiency ‘E’ is given by the expression R=(1/E-1) 1/6 R₀, R₀ is the distance between donor and acceptor for 50% energy transfer efficiency.

Forster Radius R0 is expressed as R₀=[8.8×1023×k2xn4×QYD×J(λ)]1/6 A0 Where k2=dipole orientation factor, n=Refractive index, EA=Extinction coefficient of acceptor QYD=Fluorescence quantum yield of the donor in the absence of the acceptor J(λ)=Spectral overlap integral=Integral [εA(λ). FD(λ).(λ)4 d(λ)] cm3 M Where FD=Fluorescence emission intensity of donor as a fraction of the total integrated intensity and A0=Angstrom unit.

Forster radius R₀ is long when the spectral overlap or spectral overlap integral is less, i.e., the emission maximum of donor fluorophore and the excitation maximum of the acceptor fluorophore/quencher are not very close, and is short when the spectral overlap or spectral overlap integral is more, i.e., emission maximum of the donor fluorophore and the excitation maximum of the acceptor fluorophore/quencher are very close. Foster radii normally fall in the range of 22-75 Angstrom. Forster radii for many donor fluorophore and acceptor fluorophore pairs are available in public domain literatures (Penguang Wu et al, Analytical Biochemistry vol—218, pages 1-13, 1994, Robert H Fairclough et al Methods in Enzymology, vol—48, pages—347-379, 1978) or in catalogue/Hand book, or website of the suppliers of donor fluorophore and acceptor fluorophore/quencher dyes, particularly that of, Molecular Probes. Further, newer donor fluorophores and acceptor fluorophores/quenchers are being constantly developed. Donor fluorophore and acceptor fluorophore/quencher molecules are mostly hydrophobic and have the tendency to collapse on each other when they come in proximity resulting in static or contact quenching interaction in addition to the FRET interaction. Further, single stranded oligonucleotides form secondary structures and folds back. As a result, in close distance two energy transfer mechanisms (FRET and Static/contact quenching) are at work, measurement of R₀ values in case of oligonucleotides labelled with donor fluorophore and acceptor fluorophore/quencher will dependent on the length and type of the linker used for linking the donor fluorophore and acceptor fluorophore/quencher to the oligonucleotide. Because of this correct or better estimate of the R₀ value will require energy transfer efficiency ‘E’ measurement towards the 2R₀ distance to exclude any contribution of static/contact quenching energy transfer. It may be convenient to measure the quenching of any donor fluorophore and acceptor fluorophore/quencher pair by setting a particular separation between two moieties in a double stranded structure that will not have secondary structure or folding back.

No of bases separating the 3′end of the donor fluorophore labelled primer/probe and the base carrying the donor fluorophore, plus the no of bases separating the 3′ end of the acceptor/quencher labelled primer(s) and the base carrying the acceptor fluorophore/quencher, to avoid signal generation from the non-specific primer dimer or primer dimer like amplification products will usually fall within the range of 6-35 bases, for example, 8-30 bases and as a further example, 10-25 bases, depending on the donor fluorophore-acceptor fluorophore/quencher pair and the extent of overlap of 3′ ends of donor and acceptor fluorophore/quencher labelled primer pairs or primer probe pairs taking maximum six base overlaps between 3′ ends of labelled primers and labelled primer and probe. In case base overlap between 3′ ends of labelled primer(s) and labelled primer and probe is more than six bases the longer end of the range 6-35 will extend further.

When in addition to the donor fluorophore of a donor fluorophore labelled primer, a quencher or a 4-8 bases non-target sequence is also placed at the 5′ end of the donor fluorophore labelled primer/probe, no of bases separating 3′end of the donor fluorophore labelled primer/probe and the base carrying the donor fluorophore, plus the no of bases separating 3′ end of the acceptor/quencher labelled primer and the base carrying the acceptor fluorophore/quencher, to avoid signal generation from the non-specific primer dimer or primer dimer like amplification products, is about 5-25 bases, for example, 10-20 bases depending on the donor fluorophore-acceptor fluorophore/quencher pair and the overlap of 3′ ends of donor and acceptor fluorophore/quencher labelled primer pairs or primer probe pairs.

Another objective is to apply nested nucleic acid amplification to detect a target nucleic acid where a first primer pair amplifies a first segment of the target nucleic acid, a second primer pair amplifies a second segment of the first segment. Either the second primer pair is the donor fluorophore and acceptor fluorophore labelled libra primer pair of embodiments of the invention or one or both members of the second primer pair are labelled with an acceptor fluorophore or quencher and additionally a probe labelled with a donor fluorophore that hybridizes to the second segment is used, the donor fluorophore labelled probe and the acceptor fluorophore or quencher labelled second primer pair are the donor fluorophore moiety labelled probe and the acceptor fluorophore/quencher labelled primer(s) (libra primer-probe pairs) of embodiments of the present invention as described supra. Further, the first primer pair can also be provided labelled with acceptor fluorophore/quencher. Additionally, a third primer pair can also be employed to amplify a bigger segment of the target nucleic acid from which the first segment can be amplified. Further, the amplification reaction can be carried out in one step or in two steps, where amplicon generated in first step is amplified in second step.

Another objective is to apply semi-nested nucleic acid amplification to detect a target nucleic acid where a first primer and a second primer amplify a first segment of the target nucleic acid and a third primer with the first primer amplify a second segment of the first segment. Either the first and third primers are the donor fluorophore and acceptor fluorophore or quencher labelled libra primer pair of embodiments of the invention or one or both of the first and third primers are labelled with an acceptor fluorophore or quencher and additionally a probe labelled with a donor fluorophore that hybridizes to the second segment is used, the probe and the first and third primers are the donor fluorophore moiety labelled probe and the acceptor fluorophore/quencher labelled primer(s) (libra primer-probe pairs) of embodiments of the present invention as described supra. Further, the amplification reaction can be carried out in one step or in two steps, where amplicon generated in first step is amplified in second step.

Another objective is to detect a nucleic acid or a non-nucleic acid target including but not limited to protein, antigen, antibody, lipid, glycosylated biomolecules, cells live or dead, cancer cells, stem cells, very small embryonic like stem cells, cancer stem cells, cancer protein and DNA markers, methylated DNA, transcription factors, cytokines, carbohydrates/sugars, small molecules like haptens, where a first binding moiety with very high affinity for the nucleic acid or the non-nucleic acid target is used to capture the nucleic acid or the non-nucleic acid target and a second binding moiety that can be the same first binding moiety or a different binding moiety with very high affinity for the nucleic acid or non-nucleic acid target is used to bind to the captured nucleic acid or the non-nucleic acid target, or a third binding moiety that binds to the second binding moiety with very high affinity is used. The second or the third binding moiety is provided appended with a synthetic or natural nucleic acid target molecule that can be detected by nucleic acid amplification using donor fluorophore and acceptor fluorophore/quencher labelled libra primer pair or libra probe and primer pair of embodiments of the present invention after washing out the unbound nucleic acid appended second or third binding moiety.

The binding moieties can be selected from the group but not limited to the binding pairs antigen-antibody, protein-anti-protein antibody, antibody—antibody, antibody-anti-IgG antibody, first antibody-second antibody, Protein A-antibody, Protein G-antibody, biotin—avidin, biotin—streptavidin, lectin-sugar, nucleic acid—nucleic acid, protein—nucleic acid, protein—protein, aptamer—aptamer, aptamer-nucleic acid, aptamer—protein, hapten—anti-hapten antibody where the haptens are the small molecules including but not limited to the fluorescent dyes, bromo-d-UTP, aflatoxins and other mycotoxins, peptides, sugars.

Another objective is to use a binding moiety to capture a cell or a target nucleic acid (methylated or unmethylated), where the binding moiety is an antibody, protein, biotin, avidin, streptavidin, aptamer or a nucleic acid and target nucleic acid of the captured cell or the captured target nucleic acid is detected with or without purification by nucleic acid amplification using donor fluorophore and acceptor fluorophore/quencher labelled libra primer pair or libra probe and primer pair of embodiments of the present invention.

Another objective is to use a first donor fluorophore labelled first primer, an acceptor fluorophore / quencher labelled second primer and a second donor fluorophore labelled probe where the positions of the first donor fluorophore and the acceptor fluorophore/quencher on two primers are so selected that the first donor fluorophore generate a first fluorescence signal on target amplification. The first donor fluorophore and acceptor fluorophore/quencher labelled primers are libra primer pair of embodiments of the invention.

Further, the first and the second donor fluorophores and the acceptor fluorophore/quencher are so selected and their positions on primers and probe are so selected that the second donor fluorophore of the probe generate a second fluorescent signal on hybridization of the second donor fluorophore labelled probe to the target amplification product. Second donor fluorophore labelled probe, first donor fluorophore labelled primer and acceptor fluorophore/quencher labelled primer are libra primer-probe pair of embodiments of the invention, and either the first donor fluorophore is a donor for the second donor fluorophore or, for example, the second donor fluorophore is a donor for the first donor fluorophore.

Another objective is to use a donor fluorophore labelled primer and an acceptor fluorophore labelled primer to amplify a target nucleic acid sequence. The donor fluorophore and the acceptor fluorophore labelled primers are so selected and so labelled that there is 20-70 percent for example, 30-40 percent energy transfer between the donor and the acceptor in the target amplification product. The separation between the donor fluorophore and the acceptor fluorophore in target amplification is for example 8 to 25, as a further example, 12-20 bases depending on the spectral properties of the donor fluorophore and acceptor fluorophore pair so that the target amplification product generates a melting curve or melting temperature well separated from the same of the primer dimer. The donor fluorophore is excited and the emission of the acceptor fluorophore (FRET signal) is measured to get an estimate of the target amplification.

The no of bases separating the donor fluorophore labelled base and the 3′ end of the donor fluorophore labelled primer, plus the no of bases separating the acceptor fluorophore labelled base and the 3′ end of the acceptor fluorophore labelled primer, minus the possible number of base overlaps between the 3′ ends of two primers is plus-minus 3, for example, plus-minus 1, 0 or as a further example, the two labelled bases are placed opposite to each other in primer dimer so that there is static or contact quenching between donor fluorophore and acceptor fluorophore where there is energy transfer from the donor fluorophore to the acceptor fluorophore resulting in quenching of the donor and at the same time there is quenching of the acceptor fluorophore emission due to static or contact quenching resulting in nil or near nil signal from primer dime

In another extension of this embodiment instead donor fluorophore and acceptor labelled primer pair, a donor fluorophore labelled probe and an acceptor fluorophore labelled primer is used with same specification.

Another objective is to use a donor fluorophore labelled primer and an acceptor fluorophore/quencher labelled primer of embodiments of the invention in allele specific amplification, for example, allelic PCR where one of the primers is allele specific with its 3′ end penultimate base is the allelic base (mutated base being addressed) and the donor fluorophore or the acceptor fluorophore/quencher labelled base of the allelic primer is a thymine base 2 to 5 bases for example, 3 to 4 bases away from it's 3′ end, further the donor fluorophore or acceptor fluorophore/quencher labelled T base may have G to T or C to T or A to T, for example, G to T or C to T base mismatch with the corresponding base of the target sequence for better discrimination between two alleles. Further, the above two labelled primers are the donor fluorophore and acceptor fluorophore labelled libra primers of embodiments of the invention.

In another extension, a donor fluorophore labelled probe is used in conjunction with an acceptor fluorophore/quencher labelled primer of embodiments of the invention are used where the acceptor fluorophore/quencher labelled primer is, for example, the allele specific primer and has same specifications as described above and the labelled probe and primer are the donor fluorophore labelled and acceptor fluorophore/quencher labelled libra probe-primer pair of embodiments of the invention.

Another aspect of embodiments of the present invention is to provide at least a positive control template(s) plus at least a donor fluorophore labelled primer and an acceptor fluorophore/quencher labelled primer [libra primer pair] or at least a donor fluorophore labelled probe and an acceptor fluorophore/quencher labelled primer(s) [libra primer-probe pair(s)] of embodiments of the invention for the positive control template or templates.

Another aspect is to employ multiplexing using multiple donor fluorophore labelled and acceptor fluorophore/quencher labelled libra primer pairs or multiple donor fluorophore labelled and acceptor fluorophore/quencher labelled libra probe and primer pairs of embodiments of the invention for detection and/or quantification of multiple target sequences.

Another aspect is to amplify at least one target sequence using one non-target primer in conjunction with a target specific primer or two non-target primers, where non-target primer sequence or sequences are appended or incorporated at one or two ends of the target sequence by any of the known methods in the art and the combination of one target specific primer and one non-target primer as well as the two non-target primers are donor fluorophore and acceptor fluorophore labelled libra primer pair of embodiments of the invention. Further, the non-target sequence or sequences can be appended to the target sequence or sequences using any of the known method in the art without any limitation including PCR primer or primers appended with non-target sequence or sequences at their 5′ ends and extending them in a polymerisation or PCR reaction (including like Tail PCR reaction) or hybridizing a first target specific single stranded oligonucleotide carrying at its 5′ end sequence of the first non-target sequence and a second target specific single stranded oligonucleotide carrying at its 3′ end sequence of the second non-target sequence to one strand of the target sequence and ligating the first and the second target specific oligonucleotides using the ligase enzyme (two target specific single stranded oligonucleotides are two consecutive sequences and one of them has a phosphate group for ligation) or extending using a polymerase a first single stranded sequence carrying at its 5′ end a first non-target sequence and having a few bases overlap at its 3′ end with 3′ end of a single stranded RNA or micro-RNA or m-RNA or single or double stranded DNA target and similarly extending a second single stranded sequence carrying at its 5′ end a second non-target sequence and having a few bases overlap at its 3′ end with 3′ end of the above extension product (over the single stranded RNA or the DNA target),

Further, in tail PCR, a specific objective is to use at a fraction of normally required primer concentration, two tail primers each comprising an amplification primer or priming sequence and a tail sequence at 5′ end of the primer or priming sequence, where the tail sequence is not a target specific sequence, to initiate a target amplification and to drive the target amplification using another pair of primer corresponding to the two non-target tail sequences. The primer pair corresponding to the two tail sequences are the donor fluorophore and acceptor fluorophore/quencher labelled libra primer pairs of embodiments of the invention.

A further objective is to provide the donor fluorophore moiety labelled non-target primer described above quenched by additionally providing an acceptor fluorophore/quencher or 4-8 non-target sequence at 5′ end of the same.

Another objective is to use a first non-target primer carrying at its 5′ end a promoter sequence and at its 3′ end a poly T sequence followed with or without, for example, with one or two non-thymine bases and a second target specific primer to amplify a target where the first non-target primer and the second target specific primer are donor fluorophore and acceptor fluorophore labelled libra primer pairs of embodiments of the invention. The first non-target primer gets extended over the target sequence (RNA /DNA, for example, messenger RNA, m-RNA), the second target specific primer sits on the extended strand and gets extended by a polymerase or polymerases including reverse transcriptase and DNA Polymerase to generate a template for RNA polymerase to transcribe RNA sequence and RNA is transcribed. Primer extensions and RNA transcription are cycled repeatedly and the target sequence is linearly amplified. In another extension a double stranded adaptor with the promoter sequence and a few bases 3′ end protrusion is used in place of the above first non-target primer sequence. Target DNA can be single or double stranded.

In a further extension, a donor fluorophore labelled probe is also used and the labelled probe and primer(s) are the libra primer-probe pairs of embodiments of the invention.

Another objective is to covalently attach a donor fluorophore or acceptor fluorophore/quencher labelled primer or a donor fluorophore labelled probe (through their 5′ ends or 3′ ends or an internal base/internal link) to a solid surface like glass or glass wafer or plastic (transparent or translucent) or well or spot and provide other primer or primers in the reaction mixture in contact with the solid surface of the reaction chamber and subject to nucleic acid amplification. A large array of the labelled primers or probes for a single nucleic acid target sequence or multiple nucleic acid target sequences can be covalently attached to the solid surface of the reaction chamber or chambers [well(s)] for the detection of a single or multiple nucleic acid targets.

Another objective is to use a donor fluorophore and an acceptor fluorophore/quencher labelled libra primer pair or a donor fluorophore labelled and acceptor fluorophore/quencher labelled libra probe-primer pair of embodiments of the invention for absolute quantification of a target sequence or sequences.

One aspect of absolute quantification of nucleic acid target by amplification is to apply Poisson distribution to the nucleic acid amplification reaction. For applying Poisson distribution, the probability of the presence of a single target sequence in an amplification reaction should be less than 1 and for the same amplification reaction mixture is divided into thousands of droplets of nanolitre or less volume and the amplification reaction signal for each droplet is measured at the end point of the amplification reaction in a sharp contrast to the measurement of signal at logarithmic phase of the amplification used in PCR or qPCR. This is known as digital or droplet PCR. In digital PCR, problem is failure of large number of amplification reactions due to the use of end point measurement and use of less quantity of primers or primers and probe for that. As a result, 20000 to 40000 droplets are generated for a good quantification and this requires specialized expensive equipment. Use of libra primer pair or libra probe-primer pair would allow use of more primers and probe, hence less reaction failures and hence use of a smaller number of droplets or smaller number of very small volume reactions for arriving at a good absolute quantification of target sequence or sequences.

In certain embodiments methods of synthesizing nucleic acid molecules are provided where such methods involve contacting a target nucleic acid sequence with a mixture of at least one donor fluorophore labelled primer and acceptor fluorophore or quencher labelled primer, one or more nucleoside and/or deoxy nucleoside triphosphates, a polymerase thermostable or non-thermostable, wherein signal from non-specific primer dimer products is eliminated or almost eliminated. In certain embodiments methods of synthesizing nucleic acid molecules are provided where such methods involve contacting a target nucleic acid sequence with a mixture of at least one donor fluorophore labelled probe and one or more acceptor fluorophore or quencher labelled primers, one or more nucleoside and/or deoxy nucleoside triphosphates, a polymerase thermostable or non-thermostable, wherein signal from non-specific primer dimer like products is eliminated or almost eliminated. In certain embodiments polymerase is a DNA dependent DNA polymerase or a RNA dependent DNA polymerase.

In certain embodiments, kit or kits for performing certain of the instant methods are provided. The kit or kits comprise in one or more containers at least one primer pair labelled separately with a donor fluorophore or an acceptor fluorophore or a non-radiative quencher. In certain embodiments, kit or kits for performing certain of the instant methods are provided and the kit or kits comprise in one or more containers at least one primer pair labelled separately with a donor fluorophore or an acceptor fluorophore or a non-radiative quencher where the primer pair is a universal primer pair common for amplifying any target sequence. In certain embodiments, kit or kits comprise at least one donor fluorophore labelled probe and at least one or more acceptor fluorophore or non-radiative quencher labelled primers. Further, the kit or kits may also contain a reaction mixture containing all required components (PCR mix or PCR master mix or amplification reaction mix), one or more nucleoside and/or deoxy nucleoside triphosphates, a reaction buffer, a polymerase or polymerases, thermostable or non-thermostable or ligase, thermostable or non-thermostable, enzymes topoisomerase, recombinase, helicase, thermostable or non-thermostable, single strand binding protein or proteins, thermostable or non-thermostable. Further, the kit or kits of may also contain at least one positive control template and a labelled primer pair or labelled primer probe pair for amplifying the positive control template.

Primers and probes are oligonucleotides 10-50 bases long, for example, 15-35 bases long and, as a further example, 20-30 bases long, can be perfectly or imperfectly complementary to the target sequence, as long as the desired property resulting from complementarities, i.e., the ability to hybridize to or prime the target is not lost and may carry one or more modified bases or modified sugar moiety/moieties and may carry one or more modified base or modified sugar moieties.

The target nucleic acid is selected from natural or synthetic or semi-synthetic single or double stranded DNA or RNA, single or double stranded c-DNA, genomic DNA, methylated DNA, mitochondrial DNA, exosome DNA, plasmid DNA, ribosomal RNA (rRNA) transfer RNA (tRNA), messenger RNA (m-RNA),small RNA, including without limitation, micro-RNA, sRNA, stRNA, snoRNA, ncRNA, DNA from stem cell including very small embryonic like stem cells, viral DNA or RNA or cancer cell DNA from any source including but not limited to body fluids, biopsy samples, tumor, puss, saliva, faeces, cancer stem cell and synthetic or semisynthetic DNA or RNA, single or double stranded generated by appending one or two non-target synthetic sequences to the ends of the target nucleic acid. Further, a target nucleic acid need not constitute the entire nucleic acid molecule.

The donor fluorophore and the acceptor fluorophore/quencher label on internal base can be on any of the four bases of deoxynucleotides, deoxyuridine, for example, thymine base.

Another objective is to select the nucleic acid amplification from Polymerase chain reaction (PCR) including but not limited to quantitative PCR (qPCR), Reverse transcriptase PCR (RT-PCR), Allelic PCR, Nested PCR, Semi-nested PCR, Methylation status PCR, Emulsion PCR, Tail PCR, Droplet or digital PCR (dPCR), In-situ PCR, Isothermal Nucleic acid amplification including but not limited to Loop-mediated amplification (LAMP), Recombinase polymerase amplification (RPA), Helicase polymerase amplification (HPA), NASBA and variants of Isothermal amplification involving allelic primer or primer-probe pair, nested or semi-nested primer pair, nested or semi-nested primer-probe pair variants of Isothermal amplifications but not limited to these.

The length of the target amplification product in PCR amplification is 40-200 base pairs and, for example, 70-120 base pairs. The length of the of the target amplification product in Isothermal loop mediated nucleic acid amplification (LAMP) is 100-600 base pairs, for example, 150 to 280 base pairs and internal primers or loop primers are labelled as libra primer pair. In Recombinase polymerase amplification (RPA), length of the of the target amplification product can be as long as 1000 base pairs but 100-200 base pairs may be desired and primers are labeled as libra primer pair. In Helicase polymerase amplification (HPA), length of the target amplification product can be as long as 1000 base pairs but 100-200 base pairs may be desired.

The donor fluorophores, acceptor fluorophores and quenchers of embodiments of the invention are selected from the reporter dyes or the dyes, Fluorescein, 5-Carboxyfluorescein (5-FAM), 6-Carboxyfluorescein (6-FAM), 6-FAM (Azide), 2′7′-dimethoxy—4′5-6—carboxyfluorescein (JOE), 5-(4,6-dichlorotriazin-2 yl) Aminofluorescein (DTAF), Fluorescein isothiocyanate, HEX (Hexachlorofluorescein), TET (Tetrachlorofluorescein), VIC (Victoria Blue), MAX™ VIC with spectral profile nearly identical to VIC, SUN™ A VIC® (ThermoFisher Scientific) equivalent, TYET™563, NED, fluorescamine, Pyrene, Pyrene butyrate, succimidyl 1 Pyrene butyrate, Rhodamine (Rhod), Rhodamine 123, Rhodamine B, Sulforhodamine, 6-carboxyrhodamine (R6G), 6-carboxy-Xrhodamine (ROX), Sulforhodamine b, Sulforhodamine 101, Sulphonyl chloride derivative of sulforhodamine 101 (Texas Red), Texas Red®-X, Texas Red®-X, TEX 615 N′, N′,N′,N′-Tetramethyl—6—carboxyrhodamine (TAMRA), TAMRA™ (Azide), Rhodamine Green™-X, Rhodamine Red™, Tetramethyl rhodamine, Tetramethyl rhodamine isothiocyanate (TRITC), Terbium chelates Europium chelates, Quantum dots, graphene quantum dots, 5-(2′-aminoethyl) aminonapthylamide-3,5disulfonate(Lucifer Yellow vs), 7-amino-4-methylcoumarin (amc, coumarin 120), 7-amino-4-trifluoromethylcoumarin (coumarin 151), Cyanine dyes (sulphonate or non-sulphonated) including but not limited to Cyanine 2, Cyanine 3, Cyanine 3.5, Cyanine 5, Cyanine 5.5, Cyanine 7, [5-dimethylamino] Napthalene-1-sulfonyl chloride (DNS, Dancyl chloride), 4-(4′—dimethylaminopheylazo) benzoic acid (DABCYL), DABSYL, 4-dimethylaminopheylazophenyl)-4′-isithiocyanate (DABITC), IAEDANS (5-((((2-iodoacetyl) amino) Ethyl) amino) naphthalene sulfonic acid), EDANS, QST 7, QSY9, QSY 21, QSY 35 (QSY dyes are diaryl-rhodamine derivatives), BIODIPY Dyes including but not limited to BIODIPY FL, Alexafluor dyes including but not limited to Alexafluor 350, 488, 546, 555, 568, 594, 647dyes, ATTO dyes including but not limited to ATTO™ 488, ATTO™ 532, ATTO™ 550, ATTO™ 565, ATTO™, ATTO™ 590, ATTO™ 633, ATTO™ 647N, Yakima Yellow, LI-COR IRDyes® IRDye® 700, IRDye® 800, IRDye® 800CW, , , Lightcycler® 640, Dy 750, non-radiative quenchers including but not limited to Nanogold, Blackhole quencher 0, Blackhole quencher 1, Blackhole quencher 2, Blackhole quencher 3, Eclipse quenchers, Dark Quenchers, IDT quenchers Iowa Black™ RQ, Iowa Black FQ, ZEN™, TAO and/or ZEN®, Nano Particle quenchers, MGB Dyes.

Additional dyes can also be selected from the dyes listed in the references (Penguang Wu et al, Analytical Biochemistry vol—218, pages 1-13, 1994, Robert H Fairclough et al Methods in Enzymology, vol—48, pages—347-379, 1978), www.ncbi.nlm.nih.govsite can be searched for common FRET pairs and the Hand Book of Fluorescent Probes and Research Products of Molecular Probes. New fluorophores and quenchers are constantly being developed and can be used without any special requirement. In fact, a large number of fluorophores and quenchers are there and it would be a very big list to include all of them.

### General Methods Followed:

1) All oligonucleotides were purchased with HPLC purification from Eurogentec and other commercial vendors or can be chemically synthesized by solid phase phosphotriester chemistry.
2) Preparation of chromosomal DNA: E coli cells were grown overnight in LB medium, centrifuged at 5000 rpm for 10 minutes, washed with wash buffer (20 mM Tris-HCl pH—7.5, 50 mM NaCl, 1 mM EDTA) and centrifuged at 5000 rpm for 5 minutes.

Cell pellet was used for chromosomal DNA isolation using Qiagen chromosomal DNA purification kit as per the kit protocol. Purified DNA was estimated spectrophotometrically by measuring optical density at 260 nm.
3). Synthesis of fluorophore or quencher labelled primers and probe:

Internal donor fluorophore or acceptor fluorophore/quencher labelled primers and probe are synthesized chemically by solid phase phosphotriester chemistry in oligosynthesizer machine using four deoxynucleotide phosphoramidites and donor fluorophore or acceptor fluorophore/quencher labelled thymidine (dT) phosphoramidites (Proc. Natl. Acad. Sci. USA, 1995, vol—92, pages 4347-4351) for internal labelling.

3′ end donor fluorophore labelled probes are synthesized using donor fluorophore labelled 3′ end labelling phosphoramidites. 5′ end donor fluorophore or acceptor fluorophore/quencher or Biotin labeled probe are synthesized using donor fluorophore or acceptor fluorophore/quencher or Biotin labeled5′ end labelling phosphoramidites Donor fluorophore or acceptor fluorophore/quencher labelled primers and probe can be obtained from commercial vendors.
4) Purification of oligonucleotides:

Primers and probe used are purified by HPLC or Polyacrylamide Gel Electrophoresis (PAGE).

HPLC purification of oligonucleotides are generally carried out on C-18 Reverse phase column using linear gradient of 0.1 M triethyl ammonium acetate pH-6.5 and 0.1 M triethyl ammonium acetate pH-6.5 in 75% acetonitrile.

PAGE purification is carried out by applying desalted oligonucleotide preparation from oligosynthesizer machine to an 8% polyacrylamide gel and applying high voltage (500-1000 volt depending on gel length).

Slowest moving band is excised from the gel and purified oligonucleotide is eluted from the gel after crashing it and soaking in elution buffer (ammonium acetate buffer) or in a gel elution apparatus.

Many methods for HPLC and PAGE purification of oligonucleotide are available in the art.

Commercial vendors supplying unlabelled and labelled oligonucleotides provide required purification for the oligonucleotides when ordered for.

Different types of purifications including cartridge purification, HPLC purification or PAGE (Polyacrylamide Gel electrophoresis) purifications are available.

All labelled oligonucleotides as well as unlabelled oligonucleotides and synthetic sequences were purified by HPLC or PAGE by the vendor. [0231] 5. PCR condition for amplification (using primer pair or Primer-probe pair):

Amplification reactions were carried out in 10 or 15 ul volume of amplification reaction using 2×Kapa PCR master mix supplied by commercial vendor Kapa Corporation, 0.2 uM concentrations of labelled and unlabelled primers and labelled probe.

Alternatively, PCR reaction mix containing 20 mM Tris—HCl pH 8.3, 50 mM KCl, 1.5 mM MgCl₂, 0.2 mM of each dNTP, 0.01% gelatine, 2.0 or 3.0 units of Taq polymerase in place of commercial reaction master mix can be used.

Thermal cycling parameters used for PCR were 2 minutes initial denaturation at 95° C., followed by 45 cycles of 10 seconds denaturation at 95° C. and 45 seconds annealing at 55° C. and 15 seconds extension at 72° C.

A 20 mins final 94° C. denaturation analysis was also carried out.

PCR amplifications were carried out in a Bio Rad series 1000 cycler with CFX 384 RT PCR block.

All primers and probe were designed and analysed using primer premier or Gene script software or IDT Oligoanalyzer software in IDT website.

The advancement is further illustrated hereunder by way of following non-limiting exemplary illustrations. Following examples 1-8 illustrate the feature of embodiments of the present invention.

### Examples

### Example—1: The Distance or Separation Between Donor Fluorophore and an Acceptor Fluorophore/Quencher at which the Energy Transfer BETWEEN TWO MOIETIES IS INSIGNIFICANT

The separation between a donor fluorophore moiety and an acceptor fluorophore/quencher moiety at which energy transfer from donor fluorophore to the acceptor fluorophore/quencher is insignificant is different for different donor fluorophore and acceptor fluorophore/quencher pair. Every donor fluorophore and acceptor fluorophore/quencher pair have a respective Forster radius (R₀) (separation at which energy transfer efficiency between donor fluorophore and acceptor fluorophore/quencher is 0.5), which ranges from 22 angstroms to 75 angstroms and at 2R₀ separation between donor fluorophore and acceptor fluorophore/quencher energy transfer is negligible. Therefore, for separation beyond 2R₀ energy transfer is insignificant, corresponding separation would be between 12 bases and 40 bases. However, as PCR gives very high level of amplification a small amount of energy transfer can generate a detectable signal. In addition, the length of the linker used for attaching the donor fluorophore and the acceptor fluorophore/quencher to the oligonucleotide primers and probe also matters in deciding the actual separation between the two moieties. Moreover, donor fluorophore and acceptor fluorophore/quencher molecules are largely hydrophobic and have the tendency to interact with each other when placed close. Therefore, donor fluorophore and acceptor fluorophore/quencher pairs when selected from lower end of the R₀ value range separation should be even more than their 2R₀ distance. Therefore, it would be better to add a small additional separation to the 2R₀ separation of the donor and acceptor/quencher pair for insignificant energy transfer separation.

A separation of 40 or more bases between a donor fluorophore and an acceptor fluorophore/quencher in a target amplification reaction can be safely used without any adverse effect while designing labelled primers and probe for amplifying target sequences. In the present examples donor fluorophore FAM and BHQ1 quencher have been used at separations of more than 40 bases/base pairs in target amplification product.

### Example 2: Measurement of Fluorescence Enhancement of Donor Fluorophire Fam Labelled Primer and Probe

For this purpose, the fluorescence of the donor fluorophore FAM labelled oligonucleotide primers and probes (2.5 p mole each) were measured in 20 mM Tris-HCl Ph-8.4, 50 mM KCl and 2.0 mM MgCl₂ buffer in a total volume of 15 ul without template (F₁) and hybridizing to HPLC purified synthetic template 3, SEQ ID NO.: 18 (12.5 p mole each) by first heating at 95° C. for 2 mins and then reducing the temperature to 25° C. at a rate of 0.1° C. per second having complementarity to these oligonucleotides (F₂) in Bio Rad series 1000 cycler with CFX 384 RT PCR block.

The difference in the fluorescence value between the two measurements gave the extent of fluorescence enhancement of the fluorophore FAM on hybridization to the synthetic template. Oligonucleotides used were SEQ ID NO.: 5, SEQ ID NO.: 6, SEQ ID NO.: 7, SEQ ID NO.: 8, SEQ ID NO.: 9, SEQ ID NO.: 11 and SEQ ID NO.: 12. Percentage of enhancement of donor fluorophore fluorescence on hybridization of FAM labelled oligos to synthetic template sequences was calculated from the above two fluorescent signal measurements, Enhancement=[{(F₂-F₁)/F₁}×100 percent]. Different protocols and different synthetic templates have been used. Though there were some tube to tube variations best results were considered. Measurements in spectrofluorometer would give better estimate of percentage enhancement rather than using a Real Time PCR machine.

### Results:

% Enhancements were (using Synthetic Template—3 SEQ ID NO.:18):
1) SEQ ID NO.:5—(−) 2.0% 2) SEQ ID NO.:6-17% 3) SEQ ID NO.:7-15% 4) SEQ ID NO.: 8-45% 5) SEQ ID NO.: 9-40% 6) SEQ ID NO.:11—(−) 4% 7) SEQ ID NO.: 12—(−) 28

Conclusion: The extent of enhancement of FAM fluorescence of the FAM labelled oligonucleotides are different depending on the position of the FAM labelled base at the 3′ end or the 5′ end or an internal base of the FAM labelled oligonucleotide. Further, the percentage enhancement values also depend on the purity of the HPLC purified synthetic template.

It is known that presence of G bases in the neighborhood of a fluorophore labelled base plays an important role in the quenching of fluorescence of fluorophore labelled oligonucleotides. It further depends on the base sequence, primary and secondary structure of the oligonucleotide, local sequence and linker length and there is a change when the labelled oligonucleotide hybridizes to a complementary sequence or gets extended (Seidel et al, J Phys Chem, 1996, Vol-100: 5541-5553; Kelly et al, Science, 1999, Vol-283: 375-381; Nazarenko et al, Nucleic Acid Research, 2002, Vol. 30, No. 9, 2089-2195).

### Example 3: Measurement of the Extent of Quenching Between the Donor Fluorophore Fam and the Quencher Bhq1

For this purpose, the fluorescence emission of the donor fluorophore FAM labelled oligonucleotide primers and probes (2.5 p mole each) were measured in 20 mM Tris-HCl Ph-8.4, 50 mM KCl and 2.0 mM MgCl2 buffer in a total volume of 15 ul without hybridizing (F1) or hybridizing separately to a HPLC purified synthetic templates (12.5 p mole) having complementarity to these oligonucleotides, at 55° C. for 10 minutes (F2) and hybridizing separately the FAM labelled oligonucleotides (2.5 p mole each) and BHQ1 quencher labelled oligonucleotides (3.75 p mole each) together to a HPLC purified synthetic template (12.5 p mole) having complementarity to both the oligonucleotides, at 55° C. for 10 minutes(F3). Difference between F2 value and F1 value gives the enhancement. F2-F1 value gives the enhanced fluorescence value and F3-F2 gives the net quenching. And percentage quenching is calculated as [(F3−F2)/F2]×100%. Experiment was carried out on a Bio-Rad series 1000 cycles with CFX384 RT-PCR block.

There was tube to tube variation, best possible results were considered. A spectrofluorometer would be a better instrument for this measurement instead of a Real Time PCR machine. And hybridization of FAM labelled oligo with unlabelled complimentary oligo and BHQ1 labelled oligos would give better results.

Oligonucleotides and synthetic templates used and the percentage of quenching at different separations were:
1) Template 1—SEQ ID NO.:16 SEQ ID NO.: 5 and SEQ ID NO.: 4, separation—6 bases and quenching—26%,
2) Template 1—SEQ ID NO.:16 SEQ ID NO.:6 and SEQ ID NO.: 4, separation—9 bases and quenching—21%
3) Template 2—SEQ ID NO.:17, SEQ ID NO.: 5 and SEQ ID NO.: 4, separation—8 bases and quenching—23%,
4) Template 2—SEQ ID NO.:17 SEQ ID NO.:6 and SEQ ID NO.: 4, separation—11 bases and quenching—16%.

The sequences of the synthetic templates used in this experiment are Template-1 (SEQ ID NO.: 16) and Template—2 (SEQ ID NO.:17).

Relative orientation of the FAM fluorophore labelled oligonucleotides and BHQ1 quencher labelled oligonucleotides on the synthetic templates are shown in FIG. 3.

Template-1, SEQ ID NO.: 16:
5′ TTC TAC GGT TTA CCG AAT GTG A/AG AAT GGT CAC
TGG CTT ATC ACC C 3′ 3′
AAG ATG CCA AAT GGC TTA CAC T/TC TTA CCA GAG ACC
GAA TAG TGG G 5′
Template-2, SEQ ID NO.: 17:
5′ TTC TAC GGT TTA CCG AAT GTG A/AT/AG AAT GGT
CAC TGG CTT ATC ACC C 3′
3′ AAG ATG CCA AAT GGC TTA CAC T/TA/TC TTA CCA
GAG ACC GAA TAG TGG G 5′
Template-3, SEQ ID NO.: 18:
5′ CAT CAC CAA TAA ACG CCG AGA/AGA ATG GTC ACT
GGC TTA TCA CCC 3′ 3′
GTA GTG GTT ATT TGC GGC TCT/TCT TAC CAG TGA CCG
AAT AGT GGG 5′

Red—SEQ ID NO.: 4, Blue—SEQ ID NO.: 5/6, Magenta—SEQ ID NO.: 7/8/9 To show the relative alignments of the sequences of SEQ ID NO.:4, 5,6 7, 8, 9 with respect to each other when hybridized to the different templates individual sequences (5-9) have been given a colour code. SEQ ID NO.: 5 & 6 have identical sequence differing in position of label. SEQ ID NO.: 7, 8 & 9 have identical sequence differing in position of label.

### Example—4: Amplification of Threonine Synthesae Gene of the Bacterium E. Coli Using Fam and Bhq1 Labelled Primers

### Target Sequence:

SEQ ID NO.:26 gatcctctcg gcgtttattg gtgatgaaat cccacaggaa atcctggaag agcgcgtgcg cgcggcgttt gccttcccgg ctccggtcgc caatgttgaa agcgatgtcg gttgtctgga

Amplification reactions were carried out in triplicate in 15 ul or 10 ul reaction volumes using 2× kapa PCR master mix, 1 ng of E coli chromosomal DNA preparation or no DNA (as control) using 0.2 uM each of FAM labelled Forward primers Seq Ids—7, 8 and 9 and 0.2 uM of BHQ1 labelled reverse primer Seq Id 10 separately to amplify a 111 base pair segment of E coli threonine synthase gene. In any one amplification reaction set (set of reactions with template or without template) any one of the FAM labelled primers and the BHQ1 labelled reverse primer were used.

For comparison, amplification reactions were carried out in triplicate in 15 ul or 10 ul reaction volumes using same 1 ng quantity of E coli chromosomal DNA or no DNA (control reaction), 0.2 uM of Taqman probe (SEQ ID NO.: 15) carrying FAM at 5′ end and quencher BHQ1 at 3′ end, 0.6 uM each of forward primer (SEQ ID NO.:13) and reverse primer (SEQ ID NO.:14) and 2× Kapa PCR master mix to amplify a 145 base pair segment of E coli homoserine kinase gene.

SEQ ID NO.: 7, 8 and 9 have same nucleotide sequence but differ in position of the FAM label.

Heterodimer analysis of the FAM labelled forward primers and BHQ1 labelled reverse primer shows 9 base overlap of their 3′ ends with 4 bases match and heterodimer formation with ΔG=(−) 5.0 k cal/mole.

Sum of the separations of the FAM labelled base from 3′ end of the FAM labelled primers and the separations of the BHQ1 labelled base from 3′ end of the BHQ1 labelled reverse primer are respectively 21 bases, 11 bases and 14 bases and probable separations of the FAM labelled base and the BHQ1 labelled base in primer dimer are respectively 12 bases, 2 bases and 5 bases for SEQ ID NO.:7, 8 and 9.

Amplification reactions of forward primer of SEQ ID NO.:8 and reverse primer of SEQ ID NO.:10 gave average amplification Cq values 19.8 for the amplification reaction with template DNA (FIG. 3) and Cq values 0 for no template control reactions (FIG. 4). FIG. 3A and FIG. 4A are respective melt curves.

Amplification reactions of forward primer of SEQ ID NO.:9 and reverse primer of SEQ ID NO.:10 gave average amplification Cq values 19.2 for amplification reaction with template DNA (FIG. 5) and Cq values 37.4, 38.6, 40.4 and 0 for no template control reactions (FIG. 6). FIG. 5A and FIG. 6A are respective melt curves.

Amplification reactions of forward primer of SEQ ID NO.:7 and reverse primer of SEQ ID NO.:10 gave average amplification Cq values 21.8 for the amplification reaction with template DNA (FIG. 7) and Cq values 0 for no template control reactions (FIG. 8). FIG. 7A and FIG. 8A are respective melt curves.

Discussions: In case of forward primer of SEQ ID NO.:9 and SEQ ID NO.:10, the sum of the separation of the FAM labelled base from 3′ end of SEQ ID NO.:9 and the separation of the BHQ1 labelled base from 3′ end of SEQ ID NO.:10 reverse primer is 14 bases. There is a possible overlap of 9 bases to form a heterodimer of ΔG=(−) 5.0 k cal/mole (in-silico analysis) by these two primers. As a result, donor fluorophore Fam of forward primer and the quencher BHQ1 of reverse primer are expected to come in proximity with a separation of 5 intervening bases (6 inter base distance) in the non-specific primer dimer.

There is an enhancement of about 40% on hybridization of FAM labelled primer SEQ ID NO.: 9 to a complementary sequence as demonstrated in example 2. Extent of quenching required for balancing 40% enhancement is about 28.6%. The extent of quenching between the FAM fluorophore and BHQ1 quencher at a separation of 5 bases should be more than 26% as measured for separation of 6 bases (example 3) and may be less than the quenching of 28.6% required for exact balancing of the enhancement of FAM fluorescence in primer dimer. As a result, there is an effective net fluorescence emission from primer dimers resulting in signal from the primer dimer. Because of this control reactions without template gave amplification Cq values 37.4, 38.6, 40.4.and 0 using the primers SEQ ID NO.: 9 and SEQ ID NO.: 10 of embodiments of the present invention. Placement of the FAM fluorophore and the quencher BHQ1 on primers is on higher side of an optimum placement for nil signal from the primer dimers. But the signal is a near nil signal and can be used for target amplification even though it is not the best primer pair for nil signal. May be the enhancement of 40% measured for SEQ ID NO.:9 is slightly less than the actual enhancement.

Further, regarding the question of forward primer of SEQ ID NO.:9 forming the primer dimer instead of forward primer and reverse primer, amplification reactions were carried out using no template and 0.4 uM concentration of forward primer (double the normally used conc.) and no reverse primer (FIG. 9) [FIG. 9A, the melt curve], 0.1 uM concentration of forward primer and 0.2 uM conc. of reverse primer, 0.1 uM concentration of forward primer (half of usually used conc.) and 0.3 uM conc. of reverse primer (1.5 times of usually used conc) (FIG. 10, FIG. 10A is the melt curve), 0.2 uM concentration of forward primer and 0.2 uM conc. of reverse primer (normally used for labelled primers) (FIG. 12, FIG. 12A, the melt curve), . There was no formation of primer dimer in these reactions. But the amplification reaction without any template DNA and 0.2 uM (normal) concentration of forward primer and 0.3 uM (1.5 times normal conc. of reverse primer) resulted in formation of primer dimer (FIG. 11, FIG. 11A). Since double of normally used concentration of FAM labelled forward primer did not form any primer dimer, the primer dimer formed in the no template control reaction (FIG. 6) is due to the formation of primer dimer between the FAM labelled forward primer SEQ ID NO.:9 and BHQ1 labelled reverse primer SEQ ID NO.:10 and not due to homodimer formation by the FAM labelled forward primer. Further, formation of primer dimer between FAM labelled forward primer and BHQ1 labelled reverse primer can be biased against the self-dimer of FAM labelled forward primer by using FAM labelled forward primer at half the normally used concentration and increasing the concentration of BHQ1 labelled reverse primer or using FAM labelled forward primer at normally used concentration and increasing the concentration of BHQ1 labelled reverse primer. In this direction amplification reaction was carried out using FAM labelled forward primer 0.1 uM (half the normally used concentration) and 0.3 uM BHQ1 labelled reverse primer (1.5 times of normal conc.), (FIG. 13, FIG. 13A is the melt curve). There was good amplification of the target sequence without much loss of sensitivity. Whereas use of FAM labelled forward primer 0.2 uM (normally used concentration) and 0.3 uM BHQ1 labelled reverse primer (1.5 times of normal conc.), (FIG. 14, FIG. 14A is the melt curve) gave little better amplification in comparison. Therefore, FAM labelled forward primer (SEQ ID NO.:9) 0.2 uM (normally used concentration) and 0.3 uM BHQ1 labelled reverse primer (1.5 times of normal conc.) can be used for this biasing purpose without much detrimental effect and homodimer of FAM labelled primer. It should be noted that the primers were designed to bias for non-specific primer dimer formation with 4 base complementarity. These many base complementarity and ΔG=−5.0 K Cal /mole is not wanted, ΔG value for hetero dimer formation should be below −3.0 K Cal/mole.

In case of the primer pair SEQ ID NO.: 8 and SEQ ID NO.:10, the sum of separation of the FAM labelled base from 3′ end of SEQ ID NO.: 8 and separation of the BHQ1 labelled base from 3′ end of SEQ ID NO.:10 is 11 bases. There is a possible overlap of 9 bases with ΔG value—5.0 K Cal/mole for formation of heterodimer (in-silico analysis). As a result, donor fluorophore FAM of forward primer and the quencher BHQ1 of reverse primer are expected to come in proximity with a separation of 2 intervening bases in the non-specific primer dimer. There is an enhancement of about 45% on hybridization of the FAM labelled primer (Seq8) to its complementary sequence as demonstrated in example (2). Extent of quenching required for balancing 45% enhancement is about 31%.

The extent of quenching between the FAM fluorophore and BHQ1 quencher at a separation of 2 bases should be more than 26% as measured for separation of 6 bases (example 3) and more than 28.6% (for SEQ ID NO.: 9 & SEQ ID NO.: 10) and may be less than or equal to the quenching of 31% required for exact balancing of the enhancement of FAM fluorescence in primer dimer. As a result, in the primer dimer formed there has either almost balanced enhancement and quenching or marginally little more quenching resulting in no signal from the primer dimer or marginal loss of signal due to the excess quenching. It may be noted that at close separations of 2 bases and 5 bases there may not be much difference in the extent of quenching considering involvement of contact quenching in such short separations. Amplification curve (FIG. 3) and melt curve (FIG. 3A) of the amplification reaction of SEQ ID NO.:8 and SEQ ID NO.:10 BHQ2 support this. As a result, there is a slight increase in Cq value (19.8) in comparison to Cq value (19.2) of the primer pair SEQ ID NO.:9 and SEQ ID NO.: 10 indicating little lower sensitivity in comparison to SEQ ID NO.: 9 and SEQ ID NO.:10 as there is a slight net enhancement in case of the amplification with SEQ ID NO.:9 and SEQ ID NO.:10.

In case of the primer pair SEQ ID NO.:7 and SEQ ID NO.:10, the sum of separation of the FAM labelled base from 3′ end of SEQ ID NO.:7 and separation of the BHQ1 labelled base from 3′ end of SEQ ID NO.:10 is 21 bases. There is a possible overlap of 9 bases with ΔG value of −5.0 K Cal/mole for formation of heterodimer. As a result, donor fluorophore FAM of forward primer and the quencher BHQ1 of reverse primer are expected to come in proximity with a separation of 12 intervening bases in the non-specific primer dimer. There is an enhancement of about 15% on hybridization of the FAM labelled primer (SEQ ID NO.:7) to a complementary sequence as demonstrated in example (2). Extent of quenching required for balancing 15% enhancement is about 13.05%.

The extent of quenching between the FAM fluorophore and BHQ1 quencher at a separation of 12 bases should be slightly less than 16% as measured for separation of 11 bases (example 3) and which would be more than 13.05% required for exact balancing of the enhancement of FAM fluorescence in primer dimer. As a result, there is a net quenching in the primer dimer formed resulting in no signal from the primer dimer and loss of signal due to the excess quenching. Target Amplification curve (FIG. 7) and no template control amplification curve (FIG. 8) of the amplification using SEQ ID NO.:7 and SEQ ID NO.:10 support this. A gradual downward reduction of fluorescence signal or negative curvature of amplification curve can be observed after 35 cycles of no template control amplification. As a result, there is increase in Cq value (21.8) in comparison to Cq value of the primer pair SEQ ID NO.: 9 and SEQ ID NO.: (19.2) indicating lower sensitivity in comparison to Seq 9 and Seq id—10 as well as Tagman probe amplification (Cq−21.2) (FIG. 21) and FIG. 22 is no template control reaction of Tagman assay.

Further, average Cq value of 19.2 for the primer pair SEQ ID NO.: 9 and SEQ ID NO.:10 is 2.0 units less than the Cq value 21.2 of Taqman probe assay (SEQ ID NO.:13 SEQ ID NO.:14 and SEQ ID NO.: 15); lower the Cq value higher is the yield of target amplification product and higher is the detection sensitivity i.e., amplification of the primer pair SEQ ID NO.: 9 and SEQ ID NO.:10 demonstrates that target amplification using SEQ ID NO.:9 and SEQ ID NO.:10 of this disclosure is more sensitive than the Taqman assay. Usually, a Cq value difference of 2.0 units corresponds to about 5-7 fold difference in target quantity.

Cq value of 19.8 for the primer pair SEQ ID NO.: 8 and SEQ ID NO.:10 is 1.4 units less than that of Tagman probe assay (SEQ ID NO.: 13, SEQ ID NO.:14 and SEQ ID NO.: 15) and 0.6 units more than that of the primer pair SEQ ID NO.:9 and SEQ ID NO.:10. Amplification of the primer pair SEQ ID NO.:8 and SEQ ID NO.:10 demonstrates that it is more sensitive than the Taqman assay but less sensitive than the primer pair SEQ ID NO.:9 and SEQ ID NO.:10 in target amplification. Results of both primer pair (SEQ ID NO.:9 and SEQ ID NO.:10 and SEQ ID NO.:8 and SEQ ID NO.:10 are in keeping with the concept of fluorescence enhancement and quenching compensation/balancing of embodiments of the present invention.

TABLE 1

| | | | No of intervening | |
|---|---|---|---|---|
| | | | bases separating | |
| | | | donor fluorophore | |
| | | | FAM of forward | |
| | | | primer and the | |
| | | Sensitivity/ | quencher BHQ1 of | Specific Placement |
| Labelled | Amplification | Specificity | reverse primer in the | (position) of the FAM |
| Primer | reaction Cq | based on | non-specific primer | fluorophore and the |
| pair | Value | Cq value | dimer | quencher BHQ1 on primers |
| SEQ ID | 19.8 | More | 2 | 11 |
| NO.: 8 | | Sensitive / | | |
| &10 | | Most | | |
| | | specific | | |
| SEQ ID | 19.2 | Most | 5 | 14 |
| NO.: 9 & | | Sensitive, | | |
| 10 | | slightly | | |
| | | less | | |
| | | specific | | |
| SEQ ID | 21.8 | Less | 12 | 21 |
| NO.: 7 & | | Sensitive, | | |
| 10 | | most | | |
| | | specific | | |
| TaqMan | 21.2 | Moderate | — | — |
| SEQ ID | | Specific, | | |
| NO.: 13, | | Moderate | | |
| 14 & 15 | | Sensitive | | |

Further, the fluorescence enhancement in case of the primer sequence SEQ ID NO.: 9/SEQ ID NO.:8 is only 40/45% but the extent of enhancement can be even as high as 60% to 80% as reported in the literature. In that case, present Cq value difference of 2.0 units with respect to the Taqman assay, the gold standard, may increase to 3.0 to 4.0 units, which will be a huge jump in the target amplification signal as well as in the sensitivity of target detection or quantification by nucleic acid amplification using the method of this disclosure. Additionally placing a quencher at 5′ ends of FAM labelled primer would increase fluorescence enhancement, hence better sensitivity.

It can be observed that the FAM fluorophore in SEQ ID NO.:8 & SEQ ID NO.: 9 is almost at the middle of the primer sequence and in SEQ ID NO.:7 near the 5′ end of the sequence. In reverse primer SEQ ID NO.: 10 quencher BHQ1 is 5 bases away from 3′ end. It can be further observed that the quencher BHQ1 has to be brought closer to FAM fluorophore for balancing fluorescence enhancement and quenching in non-specific primer dimer product for nil or near nil signal, which requires long base overlap (9 bases) between 3′ ends of the forward primer sequence SEQ ID NO.: 8 or SEQ ID NO.: 9 and reverse primer SEQ ID NO.: 10 and placement of BHQ1 in reverse primer SEQ ID NO.: 10 near 3′ end. Higher sensitivity of target detection requires higher fluorescence enhancement and for that donor fluorophore has to be placed near the middle of the donor fluorophore labelled primer and the acceptor or quencher moiety of acceptor or quencher labelled primer has to be labelled near its 3′ end. Fluorescence enhancement of donor fluorophore is less between 1-20 percent when it is placed near 3′ end which can be seen in case of donor fluorophore FAM labelled probes SEQ ID NO.: 5 and SEQ ID NO.: 6 of example 5. When the donor fluorophore is placed between middle and 5′ end of the donor fluorophore labelled primer (SEQ ID NO.:7) which is further away from 3′ end of the fluorophore labelled primer, acceptor or quencher has to be placed near 3′ end of acceptor or quencher labelled primer and longer overlap between the fluorophore labelled forward primer (SEQ ID NO.: 7) and the BHQ1 labelled reverse primer (SEQ ID NO.: 10).

Fresh lots of labelled primers and probes would give better results. Fresh lot always performs better as these labelled oligonucleotides are not very stable unless stabilized, which has been done by some companies with propriety composition. Formation of non-specific primer dimer product is a random/stochastic phenomenon, may form one time and may not form another time. Further, formation of non-specific product is dependent on the quality of the labelled primer and probe used and presence of inhibitor. Labelled primers and probe were obtained in two different lots.

### Example 5: Amplification of Threonine Synthesae Gene of the BACTERIUM E. coli USING FAM LABELED PROBE AND BHQ1 LABELED PRIMER: Target DNA

SEQ ID—3 tggcacaaat gctgacccat attgcgggtg ataagccagt gaccattctg accgcgacct ccggtgatac cggagcggca gtggctcatg ctttctacgg tttaccgaat gtgaaagtgg

Amplification reactions were carried out in triplicate in 10/15 ul reaction volumes using 2×Kapa PCR master mix (Kapa Corporation) with 1 ng of E coli chromosomal DNA preparation or no DNA (as control) using 0.2 uM each of unlabelled Forward primer SEQ ID NO.: 1, BHQ1 labelled reverse primers SEQ ID NO. 4, SEQ ID NO.: 2 separately and FAM labelled probes SEQ ID NO.:5 and SEQ ID NO.:6 separately to amplify a 113 base pair segment of E coli threonine synthase gene. In any amplification reaction set (set of reactions with template or without template) any one of the FAM labelled probes and any one of the BHQ1 labelled reverse primers were used in combination with the forward primer Seq 1.

For comparison, amplification reactions were carried out in triplicate in 10/15 ul reaction volumes using same 1 ng quantity of E coli chromosomal DNA or no DNA (control reaction), 0.2 uM of Tagman probe (SEQ ID NO.:15) carrying FAM at 5′ end and quencher BHQ1 at 3′ end, 0.6 uM each of forward primer (SEQ ID NO.:13) and reverse primer (SEQ ID NO.:14) and 2× Kapa PCR master mix to amplify a 145 base pair segment of E coli homoserine kinase gene.

Probe sequences, SEQ ID NO.:5 and 6 have same nucleotide sequence but differs in the position of the FAM label. Reverse primers, SEQ ID NO.:4 and SEQ ID NO.:2 have same nucleotide sequence but differ in the position of the BHQ1 label. Heterodimer analysis of the FAM labelled probe and BHQ1 labelled reverse primer shows 4 base overlaps at their 3′ ends with 4 bases match at 3′ end and heterodimer formation with ΔG=(−) 5.12 k cal/mole.

Sum of the separations of the FAM labelled base from 3′ end of the FAM labelled probe, SEQ ID NO.:5 and the separations of the BHQ1 labelled base from 3′ end of the BHQ1 labelled reverse primer, SEQ ID NO.:4 is 15 bases and probable separations of the FAM labelled base and the BHQ1 labelled base in primer dimer like non-specific products is 11 bases. Sum of the separations of the FAM labelled base from 3′ end of the FAM labelled probe, SEQ ID NO.:6 and the separation of the BHQ1 labelled base from 3′ end of the BHQ1 labelled reverse primer, SEQ ID NO.:4 is 18 and probable separations of the FAM labelled base and the BHQ1 labelled base in primer dimer like non-specific products is 14. Sum of the separations of the FAM labelled base from 3′ end of the FAM labelled probe, SEQ ID NO.:6 and the separations of the BHQ1 labelled base from 3′ end of the BHQ1 labelled reverse primer, SEQ ID NO.:2 is 7 bases and probable separation of the BHQ1 labelled base from 3′ end of the BHQ1 labelled reverse primer, in primer dimer like non-specific products is 3.

Amplification reactions of the probe, SEQ ID NO.:5 and reverse primer, SEQ ID NO.:4 gave average amplification Cq value 21. 8 for the amplification reaction with template DNA (FIG. 15) and Cq values 41.07, 40.25 and 0 for no template control reactions (FIG. 16). FIG. 16A is no template control reaction melt curve.

Amplification reactions of the probe, SEQ ID NO.:6 and reverse primer of SEQ ID NO.:4 gave average amplification Cq values 20.4 for amplification reaction with template DNA (FIG. 17) and Cq values 0 for no template control reactions (FIG. 18). Two reactions were aberrant, hence ignored.

Amplification reactions of the probe, SEQ ID NO.:6 and reverse primer of SEQ ID NO.:2 gave average amplification Cq values 24.0 for the amplification reaction with template DNA (FIG. 19) and Cq values 0 for no template control reactions (FIG. 20).

Discussions: In case of the probe, SEQ ID NO.:5 and reverse primer, SEQ ID NO.: 4, the sum of the separation of the FAM labelled base from 3′ end of SEQ ID NO.:5 and the separation of the BHQ1 labelled base from 3′ end of SEQ ID NO.:4 is 15 bases. There is an overlap of 4 bases to form a heterodimer of ΔG=(−) 5.0 k cal/mole (in-silico analysis) by these two labelled probe and primer. As a result, donor fluorophore FAM of probe and the quencher BHQ1 of reverse primer are expected to come in proximity with a separation of 11 intervening bases in the non-specific primer dimer like product. No significant enhancement on hybridization of the FAM labelled probe to a complementary sequence was observed in enhancement measurement study as demonstrated in example 2. But Cq value of 21.8 for target amplification indicates there is an enhancement of fluorescence of FAM labelled probe, SEQ ID NO.: 5. The extent of quenching between the FAM fluorophore and BHQ1 quencher at a separation of 11 bases as measured (example 3) is about 16% which is more than the quenching required for balancing the enhancement of FAM fluorescence on hybridization of Probe, SEQ ID NO.: 5.

A net quenching of fluorescence emission from primer dimer like product is expected. As only one amplification curve shows a horizontal amplification curve with Cq value 0, no formation of non-specific product and three amplification curves show Cq values 36, 40.25 and 41.07 (FIG. 19) [FIG. 19A melt curve], a marginal net quenching can be thought of as well. Only one MELT curve shows a good negative peak while remaining three MELT curves do not show a positive peak either but these two MELT curves show gradual melting in negative direction without a sharp negative peak, can be marginally different from being horizontal, this may be due to the formation of non-specific product at a later cycle of the amplification reaction. Placement of the FAM fluorophore and the quencher BHQ1 on probe and primer is on less than an optimum placement for nil signal from the primer dimers or in the borderline. Though present labelling configuration can be used for target amplification even though it is not the best primer pair for nil signal. May be a base or two increase in separation between FAM and BHQ1 in primer dimer like product would result in a nil or near nil signal.

In case of the probe, SEQ ID NO.: 6 and reverse primer SEQ ID NO.:4, the sum of the separation of the FAM labelled base from 3′ end of SEQ ID NO.: 6 and the separation of the BHQ1 labelled base from 3′ end of SEQ ID NO.: 4 is 18 bases and there is a possible overlap of 4 bases between 3′ end of the FAM labelled probe and BHQ1 labelled reverse primer. As a result, donor fluorophore FAM of forward primer and the quencher BHQ1 of reverse primer are expected to come in proximity with a separation of 14 intervening bases in the non-specific primer dimer like product. There is an enhancement of about 17% on hybridization of the FAM labelled probe, SEQ ID NO.: 6 to a complementary sequence as demonstrated in example 2. Extent of quenching required for balancing 17% enhancement is about 15%. The extent of quenching between the FAM fluorophore and BHQ1 quencher at a separation of 11 bases (example 3) is 16%, separation of 14 intervening bases in this case would result in quenching, which would be more or less equal to the 15% quenching required for exact balancing of the enhancement of FAM fluorescence in primer dimer. Amplification curve (FIG. 17) and no template control amplification curve (FIG. 18) for amplification using SEQ ID NO.:6 and SEQ ID NO.: 4 support this. Moreover, no template control reaction gave Cq value 0, which indicates either nearly balance of enhancement and quenching or no formation of non-specific product.

In case of the probe, SEQ ID NO.: 6 and reverse primer, SEQ ID NO.: 2 the sum of the separation of the FAM labelled base from 3′ end of SEQ ID NO.: 6 and the separation of the BHQ1 labelled base from 3′ end of SEQ ID NO.: 2 is 7 bases. Considering possible overlap of 4 bases between 3′ ends of probe, SEQ ID NO.:6 and reverse primer, SEQ ID NO.: 2, donor fluorophore FAM of probe and the quencher BHQ1 of reverse primer are expected to come in proximity with a separation of 3 intervening bases in the non-specific primer dimer like product. There is an enhancement of about 17% on hybridization of the FAM labelled probe, SEQ ID NO.:6 on hybridization to a complementary sequence as measured and demonstrated in example 2. The extent of quenching required for balancing enhancement of 17% is 15%.

The extent of quenching between the FAM fluorophore and BHQ1 quencher at a separation of 3 bases (example 3) is expected to be more than 26% which is more than 15% quenching required for balancing enhancement and quenching. There is a net quenching in primer dimer like non-specific product, which reduces sensitivity of target detection. Target amplification curve (FIG. 19) and no template control amplification curve (FIG. 20) support this. It may be noted that no template control amplification curve shows a negative slope after 35 cycles.

Fresh lots of labelled primers and probes would give better results. Fresh lot always performs better as these labelled oligonucleotides are not very stable unless stabilized, which has been done by some companies with propriety composition. Placement of FAM in FAM labelled probes of this example has been at or near 3′ end of the probe. Placement of FAM in FAM labelled probes further away from 3′ end of the probe would give higher enhancement of FAM fluorescence and consequently higher sensitivity of target detection. Formation of non-specific primer dimer product is a random/stochastic phenomenon, may form one time and may not form another time. Further, formation of non-specific product is dependent on the quality of the labelled primer and probe used and presence of inhibitor.

It can be observed that the FAM fluorophore is at 3′ end in the probe Seq 5 or 4 bases away from the 3′ end of the probe Seq 6 and BHQ1 quencher is 15 bases away for 3′ end in quencher labelled primer Seq 4 and 5 bases away from 3′ end of BHQ1 labelled reverse primer Seq 2. Therefore, in case of placement of fluorophore at or near 3′ end fluorescence enhancement of hybridization of the probe Seq 6 target sequence is about 17 percent. To balance enhancement on 17 percent in non-specific primer dimer like product would require 15 percent quenching of the FAM fluorophore by the quencher BHQ1 of reverse primer. In case of probe Seq 5 enhancement is much less and quenching of FAM fluorophore of Seq 5 in primer dimer like non-specific product would require much less than 15 percent quenching by BHQ1 quencher of BHQ1 labelled reverse primer. For less quenching of FAM in non-specific primer dimer like product fluorophore FAM and BHQ1 quencher has to be very far off. A separation of 11 bases between FAM and BHQ1 in non-specific product using probe Seq 5 and reverse primer Seq 4 has resulted in net quenching and there is no balance of FAM fluorescence enhancement in this non-specific product. BHQ1 label in Seq 4 is 15 bases away from 3′ end, which is between middle and 5′ end of Seq 4. Similarly, a separation of 14 bases between FAM and BHQ1 in non-specific product using probe Seq 6 and reverse primer Seq 4 has resulted in almost balanced quenching of fluorescence enhancement of FAM in non-specific product, where FAM is 4 bases away from 3′ end of Seq 6 and BHQ1 is 15 bases away from 3′ end, which is between middle and 5′ end of Seq 4. Therefore, if donor fluorophore FAM is placed at or near 3′ end of FAM labelled probe quencher BHQ1 has to be placed far off from 3′ end of the BHQ1 quencher labelled primer, between middle and 5′ end of BHQ1 labelled primer. Further, in probe SEQ ID NO.:6 FAM fluorophore is 4 bases away from 3′ end and in BHQ1 quencher labelled primer SEQ ID NO 2, BHQ1 is placed on₅th base from 3′ end. In both labelled probe and primer, labels are near the 3′ ends. There is no non-specific signal but there is large loss of target amplification signal and hence loss of sensitivity. This is the problem of placing the donor fluorophore and quencher at or near the 3′ end.

TABLE 2

| Unlabelled | | | | | |
|---|---|---|---|---|---|
| forward | | | No. of intervening | Specific | |
| Primer. | | | bases separating | Placement | |
| labelled | | | donor fluorophore | (position) of the | |
| reverse | | | FAM of forward | FAM | |
| primer | | | primer and the | fluorophore and | |
| and | | Sensitivity/ | quencher BHQ1 of | the quencher | |
| labelled | Amplification | Specificity | reverse primer in | BHQ1 on | Remark on |
| probe | reaction Cq | Based on | the non-specific | primers and | obtaining nil |
| pair | Value | Cq value | primer dimer | probes | signal |
| SEQ ID | 21.8 | Good | 11 | 15 | There is net |
| NO.: 1, | | Specificity, | | | quenching |
| 4, 5 | | Less | | | |
| | | Sensitivity | | | |
| SEQ ID | 20.4 | Very good | 14 | 18 | Enhancement |
| 1, 4, 6 | | Specificity, | | | & Quenching |
| | | good | | | are more or less |
| | | Sensitivity | | | balanced for nil |
| | | | | | signal |
| SEQ ID | 24.0 | Very High | 3 | 7 | There is very |
| NO.: 1, | | Specificity | | | strong net |
| 2, 6 | | but less | | | quenching that |
| | | Sensitivity | | | results in nil |
| | | | | | non-specific |
| | | | | | signal but loss |
| | | | | | of target signal, |
| | | | | | hence loss of |
| | | | | | sensitivity |
| SEQ ID | 21.2 | Good | — | — | — |
| NO.: 13, | | Specificity, | | | |
| 14, 15 | | Good | | | |
| TaqMan | | Sensitivity | | | |

### Example 6: Comparison of Specificity and Sensitivity Between Taqman Assay and Libra Assays

The sensitivity and specificity comparison were carried in 90 positive and 84 negative simulated clinical samples. Simulated clinical samples were prepared by spiking human nasal swab extract with E coli chromosomal DNA (2×10⁵, 2×10⁴, 2×10³, 2×10², 2×10¹, 2×10°⁰ copy number, 1 ng−10 fg DNA).

Nasal swab extract was prepared by suspending nasal swab in 20 mM Tri-HCl Ph−7.5 and adding 200 ul of chromosomal DNA extraction buffer and 200 ul of absolute ethanol each from a commercial Chromosomal DNA extraction kit to 200 ul of the nasal swab suspension and loading to a silica DNA extraction column, washing with wash buffers containing 60 percent ethanol and 80 percent ethanol and finally eluting the column with 100 ul milli Q water. Eluent is the nasal swab extract that was used for the experiment.

E coli chromosomal DNA preparation was serially diluted by 10-fold with water. 2× Kapa master mix of Kapa corporation was used for the experiment. Amplification reactions were carried out in 12.5 ul reaction volume with 0.2 uM concentration of each labelled primer and probe and 0.2 uM concentration of unlabelled primer, except 0.4 uM concentration of unlabelled primers for TaqMan assay in Bio Rad thermal cycler series 1000 CFX 384 RT-PCR machine.

Primers and probes used were SEQ ID NO.: 1, 6 and 4; SEQ ID NO.: 1, 6 and 2; SEQ ID NO.: 8 & 10 for amplification of 113 bp and 111 bp segments of E coli threonine synthase gene and SEQ ID NO.: 13, SEQ ID NO.: 14 and SEQ ID NO.: 15 for the amplification of a 145 bp segment of E coli homoserine kinase gene.

TABLE 3

| | TaqManSeq | Libra Seqs 1, 6 | Libra Seqs 8 & | Libra Seqs 1, 6 |
|---|---|---|---|---|
| | 13, 14 & 15 | & 4 | 10 | & 2 |
| Sensitivity | 75.8% | 81.3% | 86.1% | 65.4% |
| Specificity | 89.2% | 92.8% | 95.1% | 96.6% |

Discussions: SEQ ID NO.: 1 is the unlabelled forward primer and SEQ ID NO.: 2 and SEQ ID NO.: 4 are the BHQ1 labelled reverse primers for amplifying a 113 bp segment of the E coli threonine synthase gene and SEQ ID NO.: 6 is the FAM labelled common probe for these two amplification reactions. SEQ ID NO.: 13, SEQ ID NO.: 14 and SEQ ID NO.: 15 are respectively the unlabelled forward and reverse primers and FAM and BHQ1 dual labelled TaqMan probe for the amplification of a 145 bp segment of E coli homoserine kinase gene. SEQ ID NO.: 8 is the FAM labelled forward primer and SEQ ID NO.: 10 is the BHQ1 labelled reverse primer for amplifying a 111 bp segment of the E coli threonine synthase gene

The amplification reaction using SEQ ID NO.: 1 SEQ ID NO.: 4 and SEQ ID NO.: 6 gave about 5.5% higher sensitivity and 3.6% higher specificity in comparison to TaqMan assay, whereas the amplification reaction using SEQ ID NO.: 8, and SEQ ID NO.: 10 gave about 10.3% higher sensitivity and 5.9% higher specificity in comparison to TaqMan assay.

Fluorescence enhancement of FAM fluorophore of probe SEQ ID NO.:6 is less in comparison to Fluorescence enhancement of FAM fluorophore of primer SEQ ID NO.:8 and this difference has therefore resulted in a higher sensitivity in case of the amplification reaction using SEQ ID NO.: 8 and SEQ ID NO.:10 in comparison to the amplification reaction using probe SEQ ID NO.: 6 and reverse primer 4.

Further, there is relatively more quenching of FAM fluorescence by BHQ1 in non-specific amplification product in case of the primer pair SEQ ID NO.:8 and SEQ ID NO.:10 in comparison to that in case of FAM labelled probe SEQ ID NO.: 6 and BHQ1 labelled reverse primer SEQ ID NO.: 4 which is the reason for higher specificity in case of the amplification reaction of primer pair SEQ ID NO.: 8 and SEQ ID NO.:10 (sliding down of no template control curve after 35 cycles) in comparison to the amplification reaction of the probe SEQ ID NO.:6 and reverse primer SEQ ID NO. 4 (straight no template control amplification curve).

Fluorescence enhancement of FAM label of probe SEQ ID NO.: 6 is same and common for the amplification reactions involving FAM labelled probe SEQ ID NO.: 6 and BHQ1 labelled reverse primer 2 and FAM labelled probe SEQ ID NO.: 6 and BHQ1 labelled reverse primer 4. Only difference is that BHQ1 is very close to the 3′ end of the reverse primer SEQ ID NO.: 2 (5 bases away from 3′ end) in comparison to reverse primer SEQ ID NO.: 4 (16 bases away from 3′ end). The separation between FAM and BHQ1 in non-specific amplification product is much less and hence much increased quenching of the FAM fluorophore by the quencher BHQ1 in non-specific amplification product in the former case (SEQ ID NO.: 2) in comparison to that in the latter case (SEQ ID NO.: 4). This improves the specificity for the amplification reaction involving probe SEQ ID NO.: 6 and reverse primer SEQ ID NO.: 2 (specificity—96.6%) in comparison to the specificity for the amplification reaction involving probe Seq 6 and reverse primer SEQ ID NO.: 4 (specificity-92.8%). Because of this excess quenching of FAM fluorescence by BHQ1 in non-specific amplification product there is a net quenching which is more than the balanced quenching required for the generation of non-substantial signal from the non-specific amplification. This excess quenching in non-specific amplification product in this reaction reduces overall fluorescence level of this amplification reaction that reduces the signal from the target amplification product and hence reduced sensitivity. That is why there is a sizeable reduction in the target detection sensitivity in this reaction involving the labelled probe SEQ ID NO.: 6 and the labelled reverse primer SEQ ID NO.: 2 (sensitivity-65.4%) which is less than the target detection sensitivity of latter case involving the labelled probe SEQ ID NO.: 6 and the labelled reverse primer SEQ ID NO.: 4 (sensitivity-81.3%) as well as all other reactions including the TaqMan assay (sensitivity-75.8%).

SEQ ID NO.: 2 & 6 are not good BHQ1 labelled reverse primer and FAM labelled Probe combination for this target amplification.

### Example 7: Amplification of Threonine Synthesae Gene of Bacterium E Coli Using Labeled Non-Target Primers

3 femto mole each of a first oligonucleotide sequence (SEQ ID NO.: 19) carrying a first non-target primer sequence at its 5′ end and E coli threonine synthase gene sequence at its 3′ end and a second oligonucleotide sequence (SEQ ID NO.: 20) carrying E coli threonine synthase gene sequence at its 5′ end and a second non-target primer sequence at its 3′ end, where 3′ end of SEQ ID NO.: 19 has a phosphate group and threonine synthase gene sequences on first and second oligonucleotides are two consecutive sequences and designed to hybridize to one strand, were added to 1 ng of E coli chromosomal DNA in ligation buffer containing ligase enzyme and was incubated at 8° C. for 4 hrs.

Aliquots of this ligated mixture were used for PCR amplification using 3 p mole of Blackhole Quencher1 labelled first non-target primer (SEQ ID NO.: 21) and 3 p mole of FAM labelled second non-target primer (SEQ ID NO.: 22) in 15 ul reaction volumes in triplicate using 2× Kapa PCR master mix along with control reaction containing no target DNA. 63° C. annealing for 45 seconds and 15 seconds extension was used for the PCR amplification. There was good amplification of E coli threonine synthase gene sequence (FIG. 23) with Cq value of 20.4 and control reaction gave a Cq value 0 (FIG. 24).

The demonstration of example 7, is different from that of examples 4 and 5. Examples 4 and 5 are for target detection using labelled Libra primer pair and labelled Libra primer-probe pair. The libra primer-probe pairs and libra primer pairs of these two examples are different for different targets, designed and labelled separately for each target. Whereas the common non-target primer pair of example 7 are also libra primer pair but are not designed specifically for any particular target, designed specially so that same primer pair can be used for amplification of any target with higher specificity and sensitivity and at the same time the primer pair do not amplify any non-specific target sequence, the common non-target primer pairs do not have any sizeable base match with the sequence of any living organism. Primer pair SEQ ID NO.: 21 & 22 are two universal primer pairs therefore this is an alternative to DNA double strand intercalating dye based detection, which is a target detection method universal for any target, where only two unlabelled target specific amplification primers and a DNA intercalating dye are provided. This double stranded DNA intercalating dye-based detection method lacks in specificity and sensitivity, hence is not used in applications like diagnostic applications where higher specificity and sensitivity is required. Solution of example 6 is to achieve the utility of DNA intercalating dye-based detection but with higher specificity and sensitivity. In fact, labelled primer pair of example 7 is a replacement for DNA intercalating dye. This detection strategy is new, simple, cheaper, designed for use as off-the-self amplification reagent like DNA intercalating dye-based amplification reagent and particularly for superior specificity and sensitivity in comparison to DNA intercalating dye-based amplification reagent. It is a solution to the problem of lower specificity and sensitivity of DNA intercalating dye-based detection. Design requirement for the primers of this example 7 is same as that of libra primer pair and libra primer-probe pair, i.e., sum of no bases separating the base carrying donor fluorophore and 3′ end of the donor fluorophore labelled primer, plus no of bases separating the base carrying acceptor fluorophore/Quencher and 3′ end of the acceptor fluorophore/Quencher labelled primer is in the range of 6-35 bases or 6-40. Additionally, these primers are designed in such a way that these primers do not have sizeable base match with nucleotide sequence of any organism or any sequence to avoid formation of non-specific amplification products from non-target sequences.

Two primers of example 6 are libra primers used in a different strategy for a different purpose (universal reagent) and hence will result in specificity and sensitivity similar to that of libra primer pair and libra primer-probe pair. Cq value 20.4 for target amplification and Cq value 0 for no target control reaction are indications of higher sensitivity and specificity respectively, where Cq values for Taqman probe assay are respectively 21.2 and 0. The primer pair of this example 7 is not the best libra primer pair and a better labelled primer pair can be designed to achieve even better specificity and sensitivity.

In an alternative strategy, two PCR primers for a target sequence are appended separately with sequences corresponding to two labelled non-target primer sequences SEQ ID NO.: 21 and SEQ ID NO.: 22) at 5′ ends of the two PCR primers and the amplification of the target sequence is carried out with the two non-target sequence appended target amplification primers in one hundredth of normal PCR primer concentration and normal concentration of two labelled non-target primers (SEQ ID NO.: 21 and SEQ ID NO.: 22).

### Example 8: Amplification of Homoserine Kinase Gene of Bacterium E. Coli Using Labeled Primers to Generate Fret Signal

Amplification reactions were carried out in triplicate in 15 ul reaction volumes in PCR master mix with 1 ng of E coli chromosomal DNA preparation or no DNA (as control) using 3 p mole each of unlabelled forward primer (SEQ ID NO.:23, 5′-GATAAGCTGCCGTCAGAACC-3′), internal Fluorescein labelled reverse primer (SEQ ID NO.: 24, 5′—AACAGGCACTGGAGCCTAAG-3′) and internal Fluorescein labelled probe (SEQ ID NO.: 25, 5′—CCA GTG GCG ATG ACC CTG GAA AAG AAT ATG-3′) to amplify a 145 base pair segment of E. coli homoserine kinase gene. Amplification reaction was monitored by exciting fluorescein at 465 nm and measuring emission of Fluorescein at 510 nm. Target amplification reaction gave a Cq value 21.3 (FIG. 25) whereas control reaction containing no target DNA gave a Cq value 0 (FIG. 26).

In embodiments, the method or strategy of target detection in example 8 is different from that of examples 4-7, which are based on libra primer-probe pair and libra primer pair. Examples 4-7 use non-FRET signal generation for target detection where the donor fluorophore is excited and emission of donor fluorophore is measured as signal. On the contrary, a FRET signal is generated in example 8 for target detection where the donor fluorophore is excited and emission of acceptor fluorophore is measured as signal. Advantage in this strategy is fluorescent background is low and a melting curve of target amplification product distinguishable from primer dimer is generated which is an additional specificity, which is not possible in Tagman probe based detection. Sensitivity and specificity of the strategy or method of example 8 is comparable or slightly better than that of Tagman probe based target detection method but is less than that of the above three strategies of embodiments of the invention involving libra primer—probe pair and libra primer pair (examples 4-6 and 7).

In FRET based signal generation method FRET primer pair and FRET primer-probe pair are designed for maximum energy transfer (70-80 percent) between donor and acceptor for higher signal from the acceptor fluorophore. It was observed that higher signal from acceptor did not make a sizable difference in target detection sensitivity and specificity and melting curve of the target amplification product of donor and acceptor fluorophore labelled primer pair is not differentiable from that of non-specific primer dimer. Whereas the method of example 8 primers and probe are so labelled and so configured that there is only 30-50 percent energy transfer between donor fluorophore and acceptor fluorophore in target amplification product as a result, labelled primer pair or labelled primer—probe pair are further separated which results in a melting curve of the target amplification product distinguishable from that of non-specific primer dimer. Thus, an additional specificity is achieved. Additionally, primers are so labelled that in primer dimer or primer dimer like non-specific amplification products, donor and acceptor fluorophores are separated by 3 or less nucleotides, which results in nil or near nil signal from primer dimer or primer dimer like non-specific products.

Though an unlabelled primer and a fluorescein labelled primer and a fluorescein labelled probe (one fluorescein act as donor and other fluorescein act as acceptor) are used in this example 8, a donor fluorophore and an acceptor fluorophore (different from donor fluorophore) labelled primer pair can equally be used for target detection. The acceptor fluorophore may be, for example, different from the donor fluorophore. The labelled primers and probe are selected and labelled in such a way that the donor fluorophore and the acceptor fluorophore are separated by 15-25 bases in target amplification product.

Although the present invention has been disclosed in the form of embodiments and variations thereon, it will be understood that numerous additional modifications and variations could be made thereto without departing from the scope of the invention.

For the sake of clarity, it is to be understood that the use of ‘a’ or ‘an’ throughout this application does not exclude a plurality, and ‘comprising’ does not exclude other steps or elements.

1-59. (canceled)

60. A method of nucleic acid target detection and/or quantification by nucleic acid amplification including selectively controlling the extent of signal enhancement and respective attenuation of any detectable signal from nonspecific amplification and also loss of target amplification signal under controlled attenuation comprising:
providing at least one target nucleic acid, at least one oligonucleotide non-extendable or extendable, labelled with at least one shining group/moiety configured to shine extra upon hybridization or getting incorporated into a nucleic acid molecule;
providing at least one extendable oligonucleotide labelled with at least one converter or acceptor group/moiety configured to convert the color of the shining group/moiety to a different colour or thermalise it, is incorporated into an amplification product/s, and wherein the nucleic acid amplification is favourably controlled including the step of selectively controlling the extent of signal enhancement and respective attenuation of any detectable signal resulting from non-specific amplification and also any loss of target amplification signal by controlling attenuation following selectively positioning the shining/signaling moiety on shining/signaling moiety labeled oligonucleotide either on its 3′ end or on any base up to 30 bases away from the 3′ end except the 5′ end and the attenuator or acceptor moiety positioning on attenuator or acceptor moiety labelled oligonucleotide on any base at least two bases away from the 3′ end or on any base up to 30 bases away from the 3′ end and thereby ensuring controlled generation of:
(a) the target amplification product including separation of distance between donor fluorophore and acceptor fluorophore/quencher to avoid undesired interaction between the donor fluorophore and acceptor fluorophore and related undesired energy transfer in a target amplification reaction, and
(b) non-specific product under desired controlled extent of signal enhancement and respective attenuation of detectable signal ensuring the desired target amplification product with desired target detectable signal specificity resulting only from hybridizing or incorporating into a target nucleic acid amplification product the shining group/moiety of the shining group labelled oligonucleotide free of any undesirable net signal enhancement and net signal from non-specific product, net signal attenuation and loss of target amplification signal.

61. The method as claimed in claim 60, wherein the controlled generation of the non-specific product under desired controlled extent of signal enhancement and respective attenuation of detectable signal comprises following a distance of separation between the shining/signaling moiety and the attenuator or acceptor moiety labeled bases of the shining/signaling moiety and the attenuator or acceptor moiety labeled oligonucleotides in non-specific amplification product between 2R₀ distance and R₀ distance, wherein R₀ is the Forster radius of the shining/signaling moiety and the acceptor moiety pair, more specifically, the donor fluorophore and acceptor fluorophore or non-radiative acceptor/quencher moiety pair, that provides to control the extent of attenuation of signal in non-specific amplification product equal or almost equal to the extent of enhancement of signal of the shining/signaling moiety in non-specific amplification product so that there is no net signal enhancement and no net signal from non-specific product, no net signal attenuation and no loss of target amplification signal as ideal most balanced situation and in non-ideal un-balanced situation when enhancement is more than the attenuation, signalling and acceptor/attenuator moieties are separated in non-specific product by an additional 1-2 nucleotides in comparison to their separation in ideal balanced situation and when enhancement is less than the attenuation, signalling and acceptor/attenuator moieties are separated by at least up to 3 less number of nucleotides/bases in comparison to the donor acceptor separation in nonspecific product in the ideal balanced situation.

62. The method as claimed in claim 61, wherein the distance of the shining/signaling moiety labeled base from 3′ end of the shining/signaling moiety labelled oligonucleotide plus the distance of the attenuator or acceptor moiety labelled base from 3′ end of the receiver or acceptor moiety labelled oligonucleotide minus the distance of possible overlap between the 3′ ends of the signaling moiety labelled oligonucleotide and the attenuator or acceptor moiety labelled oligonucleotide comprises of the distance of separation between the signaling moiety and the attenuator or acceptor moiety labeled bases in non-specific amplification product that results in attenuation of signal equal or almost equal to the extent of enhancement of signal in the non-specific amplification product, and wherein the distance of the signalling moiety labelled base from 3′ end of the signalling moiety labelled oligonucleotide plus the distance of the attenuator or acceptor moiety labelled base from 3′ end of the attenuator or acceptor moiety labelled oligonucleotide is R₀ distance plus one internucleotide distance to 2R₀ distance plus 8 inter-nucleotide distances, wherein R₀ is the Forster radius of the signalling moiety and the acceptor moiety pair and the number of bases separating the signalling moiety labelled base from 3′ end of the signalling moiety labelled oligonucleotide plus the number of bases separating the attenuator or acceptor moiety labelled base from 3′ end of the attenuator or acceptor moiety labelled oligonucleotide is the nearest integer number of bases from R₀ (in angstrom)/3.4 angstrom (average inter-nucleotide distance) to [2R₀ (in angstrom)/3.4 angstrom (average inter-nucleotide distance)] plus up to 9 bases, wherein R₀ is the Forster radius of the signalling moiety and the acceptor/attenuator moiety pair.

63. The method as claimed in claim 61, wherein the controlled attenuation includes placement of the shining/signaling and acceptor/attenuator moieties on target amplification product more than R₀ separation between signalling/donor fluorophore and acceptor fluorophore/quencher for attaining negligible energy transfer in a target amplification reaction, and wherein the extent of signal enhancement and respective attenuation is selectively controlled to avoid undesired attenuation of the signal of the target amplification product such that for:
(i) signal enhancement of about 1-100% the respective attenuation of signal for balanced attenuation of signal from non-specific amplification product is maintained at about 1-50% maintaining separation of R₀ and 2R₀ between signalling and acceptor/attenuator moieties in non-specific amplification product; (ii) signal enhancement of about 20-80% the respective attenuation of signal for balanced signal from non-specific amplification product is maintained at about 17-45%;
(iii) signal enhancement of about 40-60% the respective attenuation of signal for balanced signal from non-specific amplification product is maintained at about 28.5-37.5%.

64. The method as claimed in claim 60, wherein the shining group/signaling moiety labeled oligonucleotide used is a non-extendable probe for monitoring nucleic acid amplification which is hybridized to one strand of the target amplification product or the target and the attenuator or converter /acceptor moiety labeled oligonucleotide used is one of the nucleic acid amplification primers which is annealed either to the same strand to which the probe hybridizes or incorporated into the strand of the target amplification product or target, to which the probe hybridizes, and wherein both amplification primers used are selectively labeled with attenuator or acceptor moiety and the shining/signalling moiety is placed on shining/signalling moiety labelled oligonucleotide either at 3′ end or on any base up to 30 bases away from the 3′ end except the 5′ end and the attenuator or acceptor moiety is placed on attenuator or acceptor moiety labelled oligonucleotide on any base at least two nucleotides away from the 3′ end or on any base up to 30 bases away from the 3′ end for the total range of signalling moiety and acceptor moiety energy transfer pairs, wherein the probes have a minor groove binding dye.

65. The method as claimed in claim 60, wherein the shining group/signaling moiety labeled oligonucleotide and the attenuator or acceptor moiety labelled oligonucleotide used are two nucleic acid amplification primers that anneal separately to two strands of the target amplification product or the target nucleic acid and get extended by polymerase or polymerases, wherein the donor fluorophore or signalling moiety is placed on the donor fluorophore/signalling moiety labelled oligonucleotide on any base at least two nucleotides away from the 3′ end or on any base up to 30 bases away from the 3′ end except the 5′ end and the attenuator or acceptor moiety is placed on attenuator or acceptor moiety labelled oligonucleotide on any base at least two nucleotides away from the 3′ end or on any base up to 30 bases away from the 3′ end for the total range of signalling moiety and acceptor moiety energy transfer pairs.

66. The method as claimed in claim 60, wherein the signaling moiety used is a donor fluorophore and the attenuator or acceptor moiety used is an acceptor fluorophore or a non-radiative acceptor or quencher moiety that accepts energy but do not emit any energy or electromagnetic radiation, signal is fluorescent signal and signal attenuation is quenching of fluorescence signal of the donor or acceptor fluorophore and the donor fluorophore and acceptor moiety is an energy transfer pair.

67. The method as claimed in claim 60, wherein the signaling moiety labelled oligonucleotide and attenuator or acceptor moiety labeled oligonucleotide used are linear.

68. The method as claimed in claim 60, wherein the shining/signaling moiety or donor fluorophore labelled non-extendable oligonucleotide is a probe and is additionally provided at 5′ end with an acceptor fluorophore or a non-radiative acceptor or quencher, or the shining/signaling moiety or donor fluorophore moiety labelled extendable oligonucleotide is a primer and is additionally provided at 5′ end with an acceptor fluorophore/quencher or a five to eight bases sequence sufficiently complementary to the sequence of the labelled primer in the vicinity of the donor fluorophore labelled base which is an internal base and forms a stem structure and are provided with or without a quencher and with or without intervening spacer between the donor fluorophore labelled primer and the five to eight bases sequence.

69. The method as claimed in claim 61, wherein the shining/signalling moiety or donor fluorophore and the acceptor are so placed on the shining moiety or donor fluorophore labelled oligonucleotide, a primer or a probe, and the acceptor or quencher moiety labelled oligonucleotide, a primer that the number of bases separating the signaling moiety or donor fluorophore moiety labeled base and the 3′ end of the donor fluorophore labelled primer or probe plus the number of bases separating the acceptor or quencher moiety labelled base and the 3′ end of the acceptor or quencher moiety labelled primer is 6-40 or 44 bases for the Forster radius range of 21 angstrom to 75 angstrom range of donor fluorophore and acceptor or quencher moiety energy transfer pairs.

70. The method as claimed in claim 61, wherein the shining moiety or donor fluorophore and the acceptor or quencher are so placed on the shining moiety or donor fluorophore labelled oligonucleotide, a primer or a probe and the acceptor or quencher moiety labelled oligonucleotide, a primer that the sum of the number of bases separating the shining or signaling moiety or donor fluorophore moiety labelled base and the 3′ end of the shining moiety or donor fluorophore labelled primer or probe plus the number of bases separating the acceptor or quencher moiety labelled base and the 3′ end of the acceptor or quencher moiety labelled primer minus the number base overlap between the 3′ ends of shining group or donor fluorophore labelled probe or primer and the acceptor/quencher labelled primer is 2 and less to bases for the donor fluorophore and acceptor or quencher moiety energy transfer pairs of the Forster radius range of 10 angstrom to 21 angstroms, wherein static or contact quenching also plays a role in addition to the Forster energy transfer in the attenuation of the shining moiety or donor fluorophore by the acceptor or quencher.

71. The method as claimed in claim 69, wherein the extent of signal enhancement or fluorescence enhancement of the donor fluorophore on incorporation of the signaling or donor fluorophore moiety labelled primer into the target amplification product or hybridization of the signaling or donor fluorophore moiety labelled probe to the target amplification product is more than 2-8-fold and extent of signal attenuation or fluorescence quenching in non-specific amplification product for non-substantial signal from such products is 65-90%, wherein the number of bases separating the signaling moiety or donor fluorophore labeled base and the 3′ end of the donor fluorophore labelled primer or probe plus the number of bases separating the acceptor or quencher moiety labelled base and the 3′ end of the acceptor or quencher moiety labelled primer is 4-16 bases for the donor and acceptor pairs of Forster radius range of 21 angstrom to 75 angstrom, the Forster radius range of the normally used donor fluorophore and acceptor FRET pairs and the signaling or donor fluorophore moiety labelled base and the acceptor or quencher moiety labelled base are separated by more than R₀ distance in the target amplification product.

72. The method as claimed in claim 60, wherein more than one signaling moiety or donor fluorophore are placed on the signaling moiety labeled probe or primer and more than one acceptor moiety or quencher are placed on the acceptor moiety labeled primer or primers.

73. The method as claimed in claim 60, wherein the shining group/moiety and converter /acceptor moiety labelled oligonucleotides are nucleic acid amplification probes and primers of nested and semi-nested nucleic acid amplification, transcription mediated nucleic acid amplification and absolute quantification of nucleic acid target.

74. The method as claimed in claim 60, wherein for amplifying at least one target nucleic acid sequence, a semi-synthetic target nucleic acid sequence is generated by appending either a first or a first and second non-target sequences respectively at one or both ends of the target nucleic acid sequence and the amplification of the target nucleic acid is driven either by one target specific primer and a primer specific for the first non-target sequence or by a primer specific for the first non-target sequence and a primer specific for the second non-target sequence respectively, wherein the target specific primer and first non-target sequence specific primer or the first and second non-target sequence specific amplification primers are the signaling or donor fluorophore moiety and acceptor or quencher moiety labelled primers, wherein the signaling or donor fluorophore moiety is placed on signaling moiety labeled oligonucleotide primer either on any base at least two nucleotides away from the 3′ end or on any base up to 30 bases away from the 3′ end except the 5′ end and the acceptor or quencher moiety is placed on acceptor or quencher moiety labelled oligonucleotide primer on any base at least two nucleotides away or on any base up to 30 bases away from the 3′ end or SEQ ID NO.:21 and SEQ ID NO.:22 labelled separately and respectively with an acceptor or quencher and a signaling or donor fluorophore moiety, wherein the signaling or donor fluorophore moiety labelled primer including selectively SEQ ID NO.:22 is additionally provided at 5′ end with an acceptor fluorophore/quencher or a five to eight bases sequence sufficiently complementary to the sequence of the labelled primer or probe in the vicinity of the fluorophore labelled base which is an internal base and forms a stem structure and are provided with or without a quencher and with or without intervening spacer between the fluorophore labelled primer and the five to eight bases sequence, and include providing a probe, wherein the probe and the primers are labelled libra primer(s) and probe,
wherein the probe is the signaling moiety or donor fluorophore labelled probe and the primers are acceptor or quencher moiety labelled primer(s), wherein the signaling or donor fluorophore moiety is placed on signaling moiety labeled oligonucleotide probe either at 3′ end or on any base up to 30 bases away from the 3′ end except the 5′ end and the acceptor or quencher moiety is placed on acceptor or quencher moiety labeled oligonucleotide primer on any base at least two nucleotides away from the 3′ end or on any base up to 30 bases away from the 3′ end.

75. A composition or a reagent or a kit for use in method of nucleic acid target detection and/or quantification by nucleic acid amplification comprising:
(a) at least a first extendable oligonucleotide labelled internally with a fluorophore or a donor fluorophore group or moiety, and (b) at least a second extendable oligonucleotide labelled internally with an acceptor or quencher moiety;
wherein the first and second oligonucleotides are two universal primers of a nucleic acid amplification reaction for amplification of any target nucleic acid sequence and the first and second oligonucleotides do not have sufficient complementarity to any known sequence of any living organism and their viruses or a fraction/portion of them and the first and second extendable oligonucleotides are the primers specific for a first and a second non-target sequences incorporated or appended to two ends of a target sequence, wherein the fluorophore or donor fluorophore moiety of the first extendable labeled oligonucleotide primer is placed on any base at least two nucleotides away from the 3′ end or on any base up to 30 bases away from the 3′ end except the 5′ end of the first extendable labeled oligonucleotide primer and the acceptor or quencher moiety of the second extendable labelled oligonucleotide primer is placed on any base at least two nucleotides away from the 3′ end or on any base up to 30 bases away from the 3′ end of the second extendable labelled oligonucleotide primer. and optionally including:
(c) an amplification reaction buffer,
(d) one or more thermostable polymerases (DNA polymerase or DNA polymerase and Reverse transcriptase or a polymerase with both DNA polymerase and reverse transcriptase enzyme activities),
(e) a DNA ligase non-thermostable or thermostable, and
(f) one or more nucleoside triphosphates.

76. The method as claimed in claim 60, wherein the shining group of the shining group labelled oligonucleotide probe or primer is a donor fluorophore and the acceptor or converter moiety of the acceptor or converter moiety labelled oligonucleotide primer is an acceptor fluorophore and the shining group labelled oligonucleotide probe or primer and acceptor or converter moiety labelled oligonucleotide primer are so selected and so labelled that the donor fluorophore labelled base and the acceptor fluorophore labelled base are separated by 5-30 bases in the target amplification product and there is a change of color or wavelength of the shine (emission of the acceptor fluorophore) in the target amplification product, wherein the changed shine, the emission of the acceptor fluorophore is the measure of target amplification; and in non-specific amplification product, shining group or donor fluorophore is attenuated or quenched by the acceptor or converter and the shining group or donor fluorophore labelled base and the acceptor or converter labelled base are separated by two or less no of nucleotides or bases in non-specific amplification product and that close contact between the donor fluorophore and the acceptor fluorophore results in static or contact quenching between the two moieties (donor fluorophore and acceptor fluorophore) and static or contact quenching results in a nil or near nil signal from the acceptor fluorophore in non-specific amplification product in spite of high energy transfer (acceptor fluorophore becomes non-fluorescent), wherein the donor fluorophore labelled oligonucleotide is either a non-extendable oligonucleotide probe and is additionally provided at 5′ end with an acceptor fluorophore or a non-radiative acceptor or quencher, or a primer and is additionally provided at 5′ end with an acceptor fluorophore/quencher or a four to eight bases sequence sufficiently complementary to the sequence of the labelled primer in the vicinity of the donor fluorophore labelled base and forms a stem structure and are provided with or without a quencher and with or without intervening spacer between the donor fluorophore labelled primer and the four to eight bases sequence.

77. The method as claimed in claim 60, wherein a shining group or donor fluorophore labelled primer and an acceptor fluorophore/quencher labelled primer, wherein the donor fluorophore labelled primer is labelled with a donor fluorophore on any base at least two nucleotides away or on any base up to 30 bases away from the 3′ end except the 5′ end and the acceptor fluorophore/quencher labelled primer is labelled with an acceptor or quencher moiety on any base at least two nucleotides away or on any base up to 30 bases away from the 3′ end are used in allele specific nucleic acid amplification, wherein one of the labelled primers is allele specific with its 3′ end penultimate base being the allelic base (mutated or changed base of the allele being addressed) and one or two bases within 2 to 5 bases away from 3′ end of the allelic primer having base mismatch with target sequence, or the donor fluorophore or the acceptor fluorophore/quencher labelled base of the allelic primer is a thymine base 2 to 5 bases away from 3′ end of the donor fluorophore or the acceptor fluorophore/quencher labelled allelic primer, and the donor fluorophore or acceptor fluorophore/quencher labelled T base has G to T or C to T or T to T, base mismatch with the corresponding base of the target sequence in addition to the penultimate base and optionally additional base mismatch for better discrimination between two alleles; alternatively a shining group or donor fluorophore labelled probe and an acceptor fluorophore/quencher labelled primer or primers are used, wherein one of the primers/acceptor fluorophore/quencher labelled primer or donor fluorophore labelled probe is used for the allele discrimination and has same specifications as described above and the shining group or donor fluorophore labelled probe is labelled with a donor fluorophore at 3′ end or any base up to 30 bases away from the 3′ end except the 5′ end and the acceptor fluorophore/quencher labelled primer or primers are labelled with acceptor fluorophore or quencher or acceptor fluorophores or quenchers on any base at least two nucleotides or bases away or on any base up to 30 bases away from the 3′ end.

78. The method as claimed in claim 60, wherein a nucleic acid molecule is amplified, wherein providing an effective amount of a first nucleic acid amplification primer complementary to a portion of first strand, an effective amount of a second nucleic acid amplification primer complementary to a portion of second strand respectively of a segment of the nucleic acid molecule, wherein first and second strands are two complementary strands of the nucleic acid, at least one or more Polymerases and/or Reverse transcriptase enzyme, reaction buffer, at least one deoxy nucleoside triphosphate, annealing the primers and synthesizing a third nucleic acid strand complementary to the first strand involving first primer sequence and a fourth nucleic acid strand complementary to the second strand involving the second primer sequence, subjecting the first and third strand and the second and fourth strand, to denaturation/strand separation, repeating the above steps of synthesis and denaturation/strand separation at least once or repeatedly, exciting the reaction mixture with a donor fluorophore exciting light or radiation and measuring a detectable signal, wherein the nucleic acid amplification is carried out using either the first and second primers, wherein the first primer is labelled with a shining group or donor fluorophore or fluorescent moiety and the second primer is labelled with an acceptor fluorophore or attenuator/quencher group or moiety, and a detectable signal is generated on target nucleic amplification, or using first and second amplification primers for amplification and hybridizing an oligonucleotide probe complementary to one of the two strands of the nucleic acid, wherein the probe is labelled with a shining group or donor fluorophore moiety and one or both primers are labelled with an acceptor fluorophore or acceptor or attenuator/quencher moiety, and a detectable signal is generated on target nucleic acid amplification.

79. The method as claimed in claim 60, wherein the nucleic acid amplification reactions comprise Polymerase Chain Reactions (PCR) or Real time Polymerase chain Reaction (RT-PCR), wherein the polymerase chain reactions (PCR) are Polymerase Chain Reaction (PCR), Reverse Transcription Polymerase Chain Reaction (RT-PCR), Allelic or Allele specific Polymerase Chain Reaction (Allelic PCR), Allelic RT-PCR, Tail PCR, Droplet PCR, Emulsion PCR, Digital PCR, Asymmetric PCR, Nested PCR, Semi—Nested PCR, methylation status PCR, in-situ PCR and the size of the target amplification product is 35 to 400 base pairs.

80. The method as claimed in claim 60, wherein the nucleic acid amplification reactions comprise Isothermal Nucleic Acid Amplifications Reactions like Loop Mediated Isothermal Nucleic Acid Amplification Reactions (LAMP), Recombinase Polymerase Amplification Reactions (RPA), Helicase Polymerase Amplification Reactions (HPA), Transcription mediated nucleic acid amplification, Nucleic acid sequence based amplification (NASBA), wherein loop primers used in LAMP facilitate DNA strand separation, strand separating enzyme like Recombinase, Helicase, Gyrase, Topoisomerase are used in RPA and HPA for denaturation or strand separation in association with single strand binding (SSB) proteins and the size of the target amplification product is 75 to 1000 base pairsand variants of isothermal amplification involving allelic primer or primer-probe pair, nested or semi-nested primer pair, nested or semi-nested primer-probe pair.

81. The method as claimed in claim 60, wherein the oligonucleotides are selectively of 10-50 bases long, sufficiently complementary to the target sequence, having the ability to hybridize or anneal and prime nucleic acid synthesis on the target and carry one or more modified bases, or modified sugar moiety / moieties, or one or more base analogues.

82. The method as claimed in claim 60, wherein a positive control template and positive control template specific labelled primer pair or labelled primer and probe pairs are additionally provided in the amplification reaction.

83. The method as claimed in claim 60, wherein multiple donor fluorophore labelled and acceptor fluorophore/quencher labelled primer pairs or multiple donor fluorophore labelled probe and acceptor fluorophore/quencher labelled primer pairs are used in a multiplexing reaction for simultaneous detection and/or quantification of multiple nucleic acid target sequences.

84. The method as claimed in claim 60, wherein one or multiple or large array of donor fluorophore/acceptor fluorophore Labelled primers or donor fluorophore/acceptor fluorophore labelled probes are attached or covalently linked or tethered through multi-carbon atom organic linker or polyethylene glycol or hybrid linker or polythymidine oligonucleotide with or without additional organic linker of sufficient length to a solid surface like glass or glass wafer or plastic like polystyrene, polyethylene, polypropylene or dextran, cellulose, nylon, transparent or translucent, microfluidic channels is used for the detection of a single or multiple or large number of nucleic acid targets in a single amplification reaction.

85. The method as claimed in claim 60, wherein the Polymerase enzyme or enzymes used for nucleic acid amplification reactions are an enzyme that is a DNA Polymerase with or without strand displacement activity or template independent primer or base extension activity or exonuclease activity in addition to polymerase activity or a Reverse Transcriptase or a Polymerase with both Reverse Transcriptase and DNA Polymerase activity or a RNA Polymerase or a RNA polymerase and DNA polymerase, natural or modified or chimeric, wherein the polymerases can be thermostable, ambient temperature or below ambient temperature active enzyme, hot start polymerase, wherein the polymerase become active after it is heated at an elevated temperature.

86. The method as claimed in claim 60 is used to detect a nucleic acid (methylated or unmethylated) or a non-nucleic acid target, wherein a first binding moiety with very high affinity for nucleic acid or non-nucleic acid target is used to capture the nucleic acid or the non-nucleic acid target and a second binding moiety that can be the same first binding moiety or a different binding moiety with very high affinity for the nucleic acid or non-nucleic acid target is used to bind to the captured nucleic acid or the non-nucleic acid target or a third binding moiety that binds to the second binding moiety with very high affinity is used, wherein the second or the third binding moiety is provided appended with a synthetic or natural nucleic acid target molecule and the bound second or third binding moiety is detected and quantified after washing out the unbound nucleic acid appended second or third binding moiety by nucleic acid amplification using donor fluorophore and acceptor fluorophore/quencher labelled primer pair or probe and primer pair; the binding moieties are selected from binding pairs antigen—antibody, protein-anti-protein antibody, antibody—antibody, antibody-anti-IgG antibody, first antibody—second antibody, Protein A-antibody, Protein G-antibody, biotin—avidin, biotin—streptavidin, lectin—sugar, nucleic acid-nucleic acid, protein-nucleic acid, peptide nucleic acid, aptamer—aptamer, aptamer—nucleic acid, aptamer-protein, hapten—anti-hapten antibody, wherein the haptens are the small molecules including but not limited to the fluorescent dyes, bromo-d-UTP, aflatoxins and other mycotoxins, peptides, sugars, wherein the donor fluorophore and acceptor fluorophore/quencher labelled primer pair or probe and primer pair are a shining group or donor fluorophore labelled primer and an acceptor fluorophore/quencher labelled primer, wherein the donor fluorophore labelled primer is labelled with a donor fluorophore on any base at least two nucleotides away and up to 30 bases away from the 3′ end except the 5′ end and the acceptor fluorophore/quencher labelled primer is labelled with an acceptor or quencher moiety on any base at least two nucleotides away and up to 30 bases away from the 3′ end or, a target specific probe labelled with a donor fluorophore at 3′ end or any base up to 30 bases away from the 3′ end except the 5′ end that hybridizes to the segment of the target sequence amplified by the primer pair of the probe and primer pair and one or both primers are labelled with acceptor fluorophores or quenchers on any base at least two nucleotides away from the 3′ end or on any base up to 30 bases away from the 3′ end, wherein a quencher may also be additionally attached at 5′ the end of the donor fluorophore labelled primer or probe.

87. The method as claimed in claim 60, wherein a method for detection and/or quantification of a large number m-RNAs or c-DNAs comprising providing first amplification primers specific for each m-RNA or c-DNA and providing as second amplification primer a common primer (common for all m-RNAs or c-DNAs in the sample) selected from a sequence appended to the m-RNAs or c-DNAs, wherein the first amplification primers and the second common amplification primer are a shining group or donor fluorophore labelled primer and an acceptor fluorophore/quencher labelled primer, wherein the shining group or donor fluorophore labelled primer is labelled with a donor fluorophore on any base at least two nucleotides away and up to 30 bases away from the 3′ end except the 5′ end and the acceptor fluorophore / quencher labelled primer is labelled with an acceptor or quencher moiety on any base at least two nucleotides away and up to 30 bases away from the 3′ end, and additionally probes specific for each m-RNA or c-DNA are provided, wherein the first amplification primers the second common amplification primer and the specific probes are labelled primer-probe pairs;
wherein the labelled probe is a probe specific for each target and labelled with a donor fluorophore at 3′ end or any base up to 30 bases away from the 3′ end except the 5′ end that hybridizes to the segment of the target sequence amplified by the first specific amplification primer and the second common primer and either the second common amplification primer or the first amplification primer is provided labelled with an acceptor fluorophore or quencher or both primers are provided labelled with acceptor fluorophores or quenchers, wherein the acceptor fluorophore or quencher is placed on acceptor fluorophore or quencher labelled primer or primers on any base at least two nucleotides away from the 3′ end or on any base up to 30 bases away from the 3′ end;
wherein optionally a quencher is additionally attached at 5′ the end of the donor fluorophore labelled primer or probe.

88. The method as claimed in claim 60, wherein the donor fluorophore labelled probe is provided attached or linked to the acceptor fluorophore or quencher moiety labelled primer through a non-nucleotide organic linker, hexamethylene, hexapolyethylene glycol or chimera or longer length of them and the probe hybridizes to the nascent nucleic strand generated through extension of the probe linked primer.

89. A kit for carrying out a nucleic acid amplification reaction comprising in one or more containers:
a) at least a donor fluorophore moiety labelled oligonucleotide probe and acceptor fluorophore or attenuator or quencher labelled oligonucleotide primer(s) including selectively, wherein the signaling moiety or donor fluorophore moiety labelled oligonucleotide probe is a probe for monitoring nucleic acid amplification which is hybridized to one strand of the target amplification product or the target nucleic acid and the acceptor fluorophore or acceptor/attenuator or quencher moiety labeled oligonucleotide primer used is one of the nucleic acid amplification primers or both primers used for amplifying the target sequence are labelled with acceptor fluorophore or acceptor or attenuator/non-radiative quencher moieties;
wherein the signaling or donor fluorophore moiety is placed on signaling or donor fluorophore moiety labeled oligonucleotide probe either on its 3′ end or on any base up to 30 bases away from the 3′ end except the 5′ end and the acceptor fluorophore or attenuator or acceptor/quencher moiety or moieties are placed on acceptor fluorophore or attenuator or acceptor moiety labeled oligonucleotide primer or primers on any base at least two bases away from the 3′ end or on any base up to 30 bases away from the 3′ end; wherein the signaling moiety used is a donor fluorophore and the attenuator or acceptor moiety used is an acceptor fluorophore or a nonradiative acceptor or quencher moiety that accepts energy but do not emit any energy or electromagnetic radiation, signal is fluorescent signal and signal attenuation is quenching of fluorescence signal of the donor fluorophore and the donor fluorophore and acceptor moiety is an energy transfer pair;
wherein the donor fluorophore labelled probe may be additionally provided labelled with an acceptor or quencher at the 5′ end of the probe
b) at least a donor fluorophore/acceptor fluorophore moiety labelled oligonucleotide probe and correspondingly an acceptor fluorophore/donor fluorophore labelled oligonucleotide primer including selectively, wherein the donor fluorophore or acceptor fluorophore moiety labelled oligonucleotide probe is a probe for monitoring nucleic acid amplification which is hybridized to one strand of the target amplification product or the target nucleic acid and the corresponding acceptor fluorophore or donor fluorophore moiety labelled oligonucleotide primer used is one of the nucleic acid amplification primers used for amplifying the target nucleic acid sequence;
wherein the donor or acceptor fluorophore moiety is placed on donor fluorophore or acceptor fluorophore moiety labelled oligonucleotide probe either on its 3′ end or on any base up to 30 bases away from the 3′ end except the 5′ end and the corresponding acceptor fluorophore or donor fluorophore moiety is placed on acceptor fluorophore or donor fluorophore labelled primer on any base at least two bases away from the 3′ end or on any base up to 30 bases away from the 3′ end,
wherein the signal is fluorescent signal and the acceptor fluorophore is the signalling moiety and the donor fluorophore and acceptor fluorophore moieties are an energy transfer pair; wherein the donor fluorophore labelled probe may be additionally provided labelled at the 5′ end of the probe with an acceptor or non-radiative quencher moiety that accepts energy but do not emit any energy or electromagnetic radiation, including selectively
c) at least a signaling or donor fluorophore labelled oligonucleotide primer and an acceptor fluorophore or acceptor/attenuator or quencher labelled oligonucleotide primer, wherein the signaling or donor fluorophore moiety labeled oligonucleotide and the attenuator or acceptor moiety labeled oligonucleotides used are two nucleic acid amplification primers that anneal separately to two strands of the target amplification product or the target nucleic acid and get extended by polymerase or polymerases;
wherein the signaling or donor fluorophore moiety is placed on signaling or donor fluorophore moiety labeled oligonucleotide primer on any base at least two nucleotides or any base away from 3′ end and up to 30 bases away from the 3′ end except the 5′ end of the signaling or donor fluorophore moiety labeled oligonucleotide primer and the attenuator or acceptor moiety is placed on attenuator or acceptor moiety labeled oligonucleotide primer on any base at least two bases away from the 3′ end or on any base up to 30 bases away from the 3′ end;
wherein the signaling moiety used is a donor fluorophore and the attenuator or acceptor moiety used is an acceptor fluorophore or a nonradiative acceptor or quencher moiety that accepts energy but do not emit any energy or electromagnetic radiation, signal is fluorescent signal and signal attenuation is quenching of fluorescence signal of the donor fluorophore and the donor fluorophore and acceptor/quencher moiety is an energy transfer pair;
wherein the donor fluorophore labelled primer may be additionally provided labelled with an acceptor or quencher at the 5′ end;
d) at least a donor fluorophore labelled oligonucleotide primer and an acceptor fluorophore labelled oligonucleotide primers, wherein the donor fluorophore moiety labelled oligonucleotide and the acceptor fluorophore moiety labeled oligonucleotides used are two nucleic acid amplification primers that anneal separately to two strands of the target amplification product or the target nucleic acid and get extended by polymerase or polymerases;
wherein the donor fluorophore moiety is placed on donor fluorophore moiety labeled oligonucleotide primer on any base at least two nucleotides or any bases away from 3′ end and up to 30 bases away from the 3′ end except the 5′ end of the donor fluorophore moiety labelled oligonucleotide primer and the acceptor fluorophore moiety is placed on acceptor fluorophore moiety labelled oligonucleotide primer on any base at least two bases away from the 3′ end or on any base up to 30 bases away from the 3′ end;
wherein the signal is fluorescent signal, signalling moiety is the acceptor fluorophore and the donor fluorophore and acceptor fluorophore moieties are an energy transfer pair;
wherein the donor fluorophore labelled primer may be additionally provided labelled with an acceptor or quencher at the 5′ end or a five to eight bases sequence sufficiently complementary to the sequence of the labelled primer in the vicinity of the donor fluorophore labelled base which is an internal base and forms a stem structure and are provided with or without a quencher and with or without intervening spacer between the donor fluorophore labelled primer and the five to eight bases sequence;
e) at least 5 donor fluorophore labelled oligonucleotide primer or primers and acceptor fluorophore or quencher labelled oligonucleotide primer or primers of including selectively of SEQ ID NO.:21-22;
f) at least donor/acceptor fluorophore labelled oligonucleotide probe or probes and acceptor fluorophore/donor fluorophore or quencher labelled oligonucleotide primer or primers;
g) at least donor fluorophore and acceptor fluorophore/quencher labelled promoter sequence carrying first primer and target specific second primer;
h) wherein the kit additionally contains in one or multiple containers at least a positive control template and positive control template specific donor fluorophore and acceptor fluorophore/quencher labelled primer pair or donor fluorophore/acceptor fluorophore/quencher labelled primer and acceptor fluorophore/donor fluorophore labelled probe,
wherein the donor fluorophore/acceptor fluorophore of the positive control template specific donor fluorophore/acceptor fluorophore moiety labeled oligonucleotide primer is placed on any base at least two nucleotides or any base away from 3′ end and up to 30 bases away from the 3′ end except the 5′ end and the acceptor fluorophore or quencher or attenuator moiety is placed on positive control template specific acceptor fluorophore or quencher/donor fluorophore moiety labeled oligonucleotide primer on any base at least two bases away from the 3′ end and on any base up to 30 bases away from the 3′ end, wherein either the signaling or donor fluorophore moiety is placed on the positive control template specific signaling or donor fluorophore moiety labeled oligonucleotide probe either on its 3′ end or on any base up to 30 bases away from the 3′ end except the 5′ end and the acceptor fluorophore or quencher moiety or moieties are placed on acceptor fluorophore or quencher moiety labeled oligonucleotide primer or primers on any base at least two bases away from the 3′ end or on any base up to 30 bases away from the 3′ end, or donor fluorophore/acceptor fluorophore moiety is placed on the positive control template specific donor fluorophore/acceptor fluorophore moiety labelled oligonucleotide probe either on its 3′ end or on any base up to 30 bases away from the 3′ end except the 5′ end and corresponding acceptor fluorophore/donor fluorophore is placed on the positive control template specific acceptor fluorophore/donor fluorophore moiety labelled oligonucleotide primer on any base at least two bases away from 3′ end or on any base up to 30 bases away from the 3′ end.
i) wherein the kit contains a labelled primer pair, wherein at least a donor fluorophore labelled probe is provided attached or linked to an acceptor fluorophore or quencher moiety labelled primer through a non-nucleotide organic linker, hexamethylene, hexapolyethylene glycol or chimera or longer length of them and the probe hybridize to the nascent nucleic strand generated through extension of the linked primer;
wherein the kit or kits as claimed may additionally include reaction buffer, plurality of deoxy nucleoside triphosphates, polymerase enzyme or enzyme, positive control template and positive template respective labelled primer pair.

90. The method as claimed in claim 60, wherein the target nucleic acid is purified, or partially purified or un-purified nucleic acid is selected from natural or synthetic or semi-synthetic single or double stranded DNA or RNA, single or double stranded c-DNA, genomic DNA, methylated DNA, mitochondrial DNA, exosome DNA, plasmid DNA, ribosomal RNA (rRNA) transfer RNA (tRNA), messenger RNA (m-RNA), small RNA, including without limitation, micro-RNA, sRNA, stRNA, snoRNA, ncRNA, DNA from stem cell including very small embryonic like stem cells, viral DNA or RNA or cancer cell DNA from any source including but not limited to body fluids, biopsy samples, tumor, puss, saliva, faeces, cancer stem cell and synthetic or semisynthetic DNA or RNA, single or double stranded generated by appending one or two non-target synthetic sequences to the ends of the target nucleic acid; wherein a target nucleic acid need not constitute the entire nucleic acid molecule and also a genomic sequence of infectious agents, mutation (single base change or deletion or insertion of a few bases or long sequences) of genomic sequence or genomic sequence of human bacteria, yeast, fungi, plant, animal, human, parasites and their viruses and any other organism, live or dead, the presence or absence of which or mutation (single base change or deletion or insertion of a few bases or long sequences) of which is implicated to the presence of disease or disorder or susceptibility to infection or disease or disorder or suitability to a disease treatment, prenatal diagnosis, genetic trait, genotype, allele type, SNP detection, cell type, tissue type, species or strain type, cancer type or sub-type, cancer detection, disease typing or sub-typing, expressed gene.


SEQUENCE LISTING (28 sequences)

SEQ ID NO: 1 - DNA, 21 bp, Artificial Sequence
  synthetic primer
gcacaaatgctgacccatatt

SEQ ID NO: 2 - DNA, 22 bp, Artificial Sequence
  Synthetic Primer with BHQ1 DYE at Base 18
tcacattcggtaaaccgtagaa

SEQ ID NO: 3 - DNA, 120 bp, Escherichia coli
  ref <308>: DATABASE ACCESSION NUMBER: GenBank / CP044355.1
  ref <309>: DATABASE ENTRY DATE: 2023-10-20
  ref <313>: RELEVANT RESIDUES IN SEQ ID NO: (877914)..(878033)
tggcacaaatgctgacccatattgcgggtgataagccagtgaccattctgaccgcgacctccggtgataccggagcggcagtggctcatgctttctacggtttaccgaatgtgaaagtgg

SEQ ID NO: 4 - DNA, 22 bp, Artificial Sequence
  Synthetic Primer - Blackhole quencher 1 on Base 7
tcacattcggtaaaccgtagaa

SEQ ID NO: 5 - DNA, 24 bp, Artificial Sequence
  Synthetic Oligonucleotide Probe - Fluorescein dye on base 24
gggtgataagccagtgaccattct

SEQ ID NO: 6 - DNA, 24 bp, Artificial Sequence
  Synthetic Olignucleotide Probe - with Fluorescein dye on base 21
gggtgataagccagtgaccattct

SEQ ID NO: 7 - DNA, 21 bp, Artificial Sequence
  Synthetic Primer - Fluorescein dye on base 3
tctcggcgtttattggtgatg

SEQ ID NO: 8 - DNA, 21 bp, Artificial Sequence
  Synthetic Primer - Fluorescein dye on base 13
tctcggcgtttattggtgatg

SEQ ID NO: 9 - DNA, 21 bp, Artificial Sequence
  Synthetic Primer - Fluorescein Dye on Base 10
tctcggcgtttattggtgatg

SEQ ID NO: 10 - DNA, 19 bp, Artificial Sequence
  Synthetic Primer - Blackhole Quencher 1 on Base 16
gacaaccgacatcgctttc

SEQ ID NO: 11 - DNA, 24 bp, Artificial Sequence
  Synthetic Oligonucleotide Probe - Fluorescein Dye on Base 1
gggtgataagccagtgaccattct

SEQ ID NO: 12 - DNA, 21 bp, Artificial Sequence
  Synthetic Primer - Fluorescein Dye on Base 1
tctcggcgtttattggtgatg

SEQ ID NO: 13 - DNA, 20 bp, Artificial Sequence
  Synthetic Primer
gataagctgccgtcagaacc

SEQ ID NO: 14 - DNA, 20 bp, Artificial Sequence
  Synthetic Primer
aacaggcactggagcctaag

SEQ ID NO: 15 - DNA, 21 bp, Artificial Sequence
  Synthetic Oligonucleotide Probe - Fluorescein Dye on Base 1 (5′ end) and BlackHole Quencher 1 on Base 21 (3′ end)
tccagggtcatcgccactgga

SEQ ID NO: 16 - DNA, 46 bp, Artificial Sequence
  Synthetic target sequence for hybridizing sequence id 5 (Probe) and sequence id 6 (Probe) and sequence id 4 (Primer) to generate different separations between Fluorescein Dye and Blackhole Quencher 1
ttctacggtttaccgaatgtgaagaatggtcactggcttatcaccc

SEQ ID NO: 17 - DNA, 48 bp, Artificial Sequence
  Synthetic target sequence for hybridizing sequence id 5 (Probe), sequence id 6 (Probe) and sequence id 4 (primer) to generate different separations between Fluorescein Dye and Blackhole Quencher 1
ttctacggtttaccgaatgtgataagaatggtcactggcttatcaccc

SEQ ID NO: 18 - DNA, 45 bp, Artificial Sequence
  Synthetic target sequence for hybridizing sequence id 7 (Primer), sequence id 8 (Primer) and sequence id 9 (Primer) to measure enhancement of Fluorescein dye
catcaccaataaacgccgagaagaatggtcactggcttatcaccc

SEQ ID NO: 19 - DNA, 65 bp, Artificial Sequence
  non-target primer sequence at 5′ end and E.coli threonine synthase gene sequence at its 3′ end
agatgtccgtgctaccagagactctcgggcggtcgctttatggcacaaatgctgacccatattgc

SEQ ID NO: 20 - DNA, 60 bp, Artificial Sequence
  E.coli threonine synthase gene sequence at 5′ end and a non-target primer sequence at its 3′ end
gggtgataagccagtgaccattctgaccgcgacctagacaccggtataacgacctgccca

SEQ ID NO: 21 - DNA, 27 bp, Artificial Sequence
  Synethic Primer - Blackhole Quencher 1 on base 23
agatgtccgtgctaccagagactctcg

SEQ ID NO: 22 - DNA, 25 bp, Artificial Sequence
  Synthetic Primer - Fluorescein Dye on Base 15
tgggcaggtcgttataccggtgact

SEQ ID NO: 23 - DNA, 20 bp, Artificial Sequence
  Synthetic Primer
gataagctgccgtcagaacc

SEQ ID NO: 24 - DNA, 20 bp, Artificial Sequence
  Synthetic Primer - Fluorescein Dye on Base 17
aacaggcactggagcctaag

SEQ ID NO: 25 - DNA, 30 bp, Artificial Sequence
  Synthetic Oligonucleotide Probe - Fluorescein Dye on Base 27
ccagtggcgatgaccctggaaaagaatatg

SEQ ID NO: 26 - DNA, 120 bp, Escherichia coli
  ref <308>: DATABASE ACCESSION NUMBER: GenBank / CP044355.1
  ref <309>: DATABASE ENTRY DATE: 2023-10-20
  ref <313>: RELEVANT RESIDUES IN SEQ ID NO: (878084)..(878203)
gatcctctcggcgtttattggtgatgaaatcccacaggaaatcctggaagagcgcgtgcgcgcggcgtttgccttcccggctccggtcgccaatgttgaaagcgatgtcggttgtctgga

SEQ ID NO: 27 - DNA, 73 bp, Escherichia coli
  ref <308>: DATABASE ACCESSION NUMBER: GenBank / CP044355.1
  ref <309>: DATABASE ENTRY DATE: 2023-10-20
  ref <313>: RELEVANT RESIDUES IN SEQ ID NO: (877974)..(878046)
ggcggtcgctttatggcacaaatgctgacccatattgcgggtgataagccagtgaccattctgaccgcgacct

SEQ ID NO: 28 - DNA, 145 bp, Escherichia coli
  ref <308>: DATABASE ACCESSION NUMBER: GenBank / CP044355.1
  ref <309>: DATABASE ENTRY DATE: 2023-10-20
  ref <313>: RELEVANT RESIDUES IN SEQ ID NO: (879003)..(879147)
gataagctgccgtcagaaccacgggaaaatatcgtttatcagtgctgggagcgtttttgccaggaactgggtaagcaaattccagtggcgatgaccctggaaaagaatatgccgatcggttcgggcttaggctccagtgcctgtt